(19) United States
(12) Patent Application Publication
(10) Pub. No.: US 2015/0099754 A1
(43) Pub. Date: Apr. 9, 2015

(54) TREATMENT OF CANCER CHARACTERIZED BY GENE MUTATIONS

(71) Applicant: SIGNAL PHARMACEUTICALS, LLC, San Diego, CA (US)

(72) Inventors: Shuichan Xu, San Diego, CA (US); Kristen Mae Hege, Burlingame, CA (US); Xiaoling Wu, Chatham, NJ (US); Zhihong Yang, Piscataway, NJ (US); Konstantinos Mavrommatis, Concord, CA (US); Ellen Filvaroff, San Francisco, CA (US)

(21) Appl. No.: 14/505,947

(22) Filed: Oct. 3, 2014

Related U.S. Application Data
(60) Provisional application No. 61/886,785, filed on Oct. 4, 2013.
(60) Provisional application No. 61/907,510, filed on Nov. 22, 2013.
(60) Provisional application No. 62/005,597, filed on May 30, 2014.

(51) Int. Cl. C07D 487/04; C12Q 1/68

(52) U.S. Cl. CPC C07D 487/04; C12Q 1/6886; C12Q 2600/106
USPC 514/249; 506/2

(57) ABSTRACT

Provided herein are methods for treating and/or preventing a cancer in a patient, comprising administering an effective amount of a TOR kinase inhibitor to a patient having cancer characterized by particular gene mutation(s) or variant(s) relative to the genes of a biological wild-type sample.

This application claims the benefit of U.S. Provisional Application No. 61/886,785, filed Oct. 4, 2013, U.S. Provisional Application No. 61/907,510, filed Nov. 22, 2013, and U.S. Provisional Application No. 62/005,597, filed May 30, 2014, the entire contents of which are incorporated herein by reference.

### 1. FIELD

Provided herein are methods for treating and/or preventing a cancer in a patient, comprising administering an effective amount of a TOR kinase inhibitor to a patient having cancer, in particular breast cancer, diffuse large B-cell lymphoma, glioblastoma multiforme, hepatocellular carcinoma, multiple myeloma, neuroendocrine tumor, or non-small cell lung cancer, characterized by particular gene mutation(s) or variant(s) relative to the genes of a biological wild-type sample.

### 2. BACKGROUND

The connection between abnormal protein phosphorylation and the cause or consequence of diseases has been known for over 20 years. Accordingly, protein kinases have become a very important group of drug targets. See Cohen, Nat. Rev. Drug Disc., 1:309-315 (2002), Grimmiger et al. Nat. Rev. Drug Disc. 9(12):956-970 (2010). Various protein kinase inhibitors have been used clinically in the treatment of a wide variety of diseases, such as cancer and chronic inflammatory diseases, including diabetes and stroke. See Cohen, Eur. J. Biochem., 268:5001-5010 (2001), Protein Kinase Inhibitors for the Treatment of Disease: The Promise and the Problems, Handbook of Experimental Pharmacology, Springer Berlin Heidelberg, 167 (2005).

The protein kinases belong to a large and diverse family of enzymes that catalyze protein phosphorylation and play a critical role in cellular signaling. Protein kinases may exert positive or negative regulatory effects, depending upon their target protein. Protein kinases are involved in specific signaling pathways which regulate cell functions such as, but not limited to, metabolism, cell cycle progression, cell adhesion, vascular function, apoptosis, and angiogenesis. Malfunctions of cellular signaling have been associated with many diseases, the most characterized of which include cancer and diabetes. The regulation of signal transduction by cytokines and the association of signal molecules with protooncogenes and tumor suppressor genes have been well documented. Similarly, the connection between diabetes and related conditions, and deregulated levels of protein kinases, has been demonstrated. See e.g., Sridhar et al. Pharm. Res. 17(11):1345-1353 (2000). Viral infections and the conditions related thereto have also been associated with the regulation of protein kinases. Park et al. Cell 101(7): 777-787 (2000).

The protein named mTOR (mammalian target of rapamycin), also called FRAP, RAFTI or RAPT1), is a 2549-amino acid Ser/Thr protein kinase, that has been shown to be one of the most critical proteins in the mTOR/PI3K/Akt pathway that regulates cell growth and proliferation. Georgakis and Younes Expert Rev. Anticancer Ther. 6(1):131-140 (2006). mTOR exists within two complexes, mTORC1 and mTORC2. While mTORC1 is sensitive to rapamycin analogs (such as temsirolimus or everolimus), mTORC2 is largely rapamycin-insensitive. Notably, rapamycin is not a TOR kinase inhibitor. Several mTOR inhibitors have been or are being evaluated in clinical trials for the treatment of cancer. Temsirolimus was approved for use in renal cell carcinoma in 2007 and everolimus was approved in 2009 for renal cell carcinoma patients that have progressed on vascular endothelial growth factor receptor inhibitors. In addition, sirolimus was approved in 1999 for the prophylaxis of renal transplant rejection. The interesting but limited clinical success of these mTORC1 inhibitory compounds demonstrates the usefulness of mTOR inhibitors in the treatment of cancer and transplant rejection, and the increased potential for compounds with both mTORC1 and mTORC2 inhibitory activity.

Somatic mutations affect key pathways in breast cancer. Accordingly, identification of specific mutations associated with breast cancer may lead to improved therapeutic protocols.

Citation or identification of any reference in Section 2 of this application is not to be construed as an admission that the reference is prior art to the present application.

### 3. SUMMARY

Provided herein are methods for treating or preventing a cancer characterized by a gene mutation, for example, breast cancer, comprising administering an effective amount of a TOR kinase inhibitor to a patient having a cancer characterized by a particular gene mutation, relative to wild type. Without being limited by theory, it is believed that certain gene mutations correlate with sensitivity to TOR kinase inhibitors, as described herein.

Further provided herein are methods for treating or preventing a cancer characterized by a gene mutation, for example breast cancer, comprising screening a patient's cancer for the presence of a particular gene mutation relative to wild type, and administering an effective amount of a TOR kinase inhibitor to the patient having a cancer characterized by a particular gene mutation.

Further provided herein are methods for predicting response to treatment with a TOR kinase inhibitor in a patient having a cancer characterized by a gene mutation, for example breast cancer, the method comprising: a) obtaining a biological test sample from the patient's cancer; b) obtaining the gene sequence of one or more genes selected from Table 1 in said biological test sample; c) comparing said gene sequence(s) to the gene sequence(s) of a biological wild-type sample; wherein the presence of a mutation indicates an increased likelihood of response to TOR kinase inhibitor treatment of said patient's cancer.

Further provided herein are methods for predicting therapeutic efficacy of TOR kinase inhibitor treatment of a patient having a cancer characterized by a gene mutation, for example breast cancer, with a TOR kinase inhibitor, the method comprising: a) obtaining a biological test sample from the patient's cancer; b) obtaining the gene sequence(s) of one or more genes selected from Table 1 in said biological test sample; c) comparing said gene sequence(s) to the gene sequence(s) of a biological wild-type sample; wherein the presence of a mutation indicates an increased likelihood of therapeutic efficacy of said TOR kinase inhibitor treatment for said patient.

Provided herein are methods for treating or preventing a cancer characterized by one or more gene variants, for example, breast cancer, diffuse large B-cell lymphoma (DLBCL), glioblastoma multiforme (GBM), hepatocellular carcinoma (HCC), multiple myeloma (MM), neuroendocrine tumor (NET), or non-small cell lung cancer (NSCLC), comprising administering an effective amount of a TOR kinase inhibitor to a patient having a cancer characterized by one or more particular gene variants, relative to wild type. Without being limited by theory, it is believed that certain gene variants correlate with sensitivity to TOR kinase inhibitors, as described herein.

Further provided herein are methods for treating or preventing a cancer characterized by one or more gene variants, for example breast cancer, DLBCL, GBM, HCC, MM, NET, or NSCLC, comprising screening a patient's cancer for the presence of one or more particular gene variants relative to wild type, and administering an effective amount of a TOR kinase inhibitor to the patient having a cancer characterized by one or more particular gene variants.

Further provided herein are methods for predicting response to treatment with a TOR kinase inhibitor in a patient having a cancer characterized by one or more gene variants, for example breast cancer, DLBCL, GBM, HCC, MM, NET, or NSCLC, the method comprising: a) obtaining a biological test sample from the patient's cancer; b) obtaining the gene sequence of the genes listed in FIG. 2 in said biological test sample; c) comparing said gene sequence(s) to the gene sequence(s) of a biological wild-type sample; wherein the presence of one or more variants in one or more genes selected from FIG. 2, Table 2, or Table 3 indicates an increased likelihood of response to TOR kinase inhibitor treatment of said patient's cancer.

Further provided herein are methods for predicting response to treatment with a TOR kinase inhibitor in a patient having a cancer characterized by one or more gene variants, for example breast cancer, DLBCL, GBM, HCC, MM, NET, or NSCLC, the method comprising: a) obtaining a biological test sample from the patient's cancer; b) obtaining the gene sequence of one or more genes selected from Table 2 or Table 3 in said biological test sample; c) comparing said gene sequence(s) to the gene sequence(s) of a biological wild-type sample; wherein the presence of one or more variants indicates an increased likelihood of response to TOR kinase inhibitor treatment of said patient's cancer.

Further provided herein are methods for predicting therapeutic efficacy of TOR kinase inhibitor treatment of a patient having a cancer characterized by one ore more gene variants, for example breast cancer, DLBCL, GBM, HCC, MM, NET, or NSCLC, with a TOR kinase inhibitor, the method comprising: a) obtaining a biological test sample from the patient's cancer; b) obtaining the gene sequence(s) of the genes listed in FIG. 2 in said biological test sample; c) comparing said gene sequence(s) to the gene sequence(s) of a biological wild-type sample; wherein the presence of one or more variants of one or more genes selected from FIG. 2, Table 2 or Table 3 indicates an increased likelihood of therapeutic efficacy of said TOR kinase inhibitor treatment for said patient.

Further provided herein are methods for predicting therapeutic efficacy of TOR kinase inhibitor treatment of a patient having a cancer characterized by one ore more gene variants, for example breast cancer, DLBCL, GBM, HCC, MM, NET, or NSCLC, with a TOR kinase inhibitor, the method comprising: a) obtaining a biological test sample from the patient's cancer; b) obtaining the gene sequence(s) of one or more genes selected from Table 2 or Table 3 in said biological test sample; c) comparing said gene sequence(s) to the gene sequence(s) of a biological wild-type sample; wherein the presence of one or more variants indicates an increased likelihood of therapeutic efficacy of said TOR kinase inhibitor treatment for said patient.

In some embodiments, the TOR kinase inhibitor is a compound as described herein.

Further provided herein are the above-mentioned TOR kinase inhibitors for use in any method described herein.

The present embodiments can be understood more fully by reference to the detailed description and examples, which are intended to exemplify non-limiting embodiments.

### 4. BRIEF DESCRIPTION OF THE DRAWINGS

FIG. 1. provides a patient disposition overview, showing treatment duration, dose modifications, and best RECIST (with target lesion response) for patients treated with Compound 1 (data as of September 2014). Signals of Compound 1 clinical activity were demonstrated with 3/17 target lesions showing PR (2/17 showing RECIST PR), all with PIK3CA mutations, in addition to mutations in RICTOR, TP53, IGF1R and/or PTEN. Additionally, mutations in BRCA2, ARID1A, FGFR1, FGFR and PTPRD were observed.

FIG. 2. provides the list of genes evaluated for variants compared to wild type.

FIGS. 3A-3C. provides a patient disposition overview, showing treatment duration, compound combination and dose modifications, EGFR mutation status, survival (in weeks) and best RECIST (with target lesion response) for NSCLC patients treated with Compound 1 and erlotinib (Arm A), Compound B and oral Azacitidine (Arm B) and Compound 1 and sequential oral Azacitidine (Arm C) (data as of September 2014). Signals of Compound 1 clinical activity were demonstrated in Arm A (FIG. 3A) with 4/25 target lesions showing PR (4/25 showing RECIST PR), in Arm B (FIG. 3B) with 1/21 target lesions showing PR (1/21 showing RECIST PR), and in Arm C (FIG. 3C) with 2/29 target lesions showing PR (2/29 showing RECIST PR).

FIG. 4. shows that low IRF4 gene expression levels correlate with sensitivity to Compound 1 in 40 hematological cancer cell lines, but not in the subset of 23 Diffuse Large B Cell Lymphoma cell lines included in the 40 cell line panel. Legend: y-axis: log 10(GI₅₀) value of Compound 1; x-axis: gene expression value of IRF4 in log 2 scale represented by probe set 216986_s_at

FIG. 5. shows that low IRF4 protein expression levels correlate with sensitivity to Compound 1 in 37 hematological cancer cell lines. Legend: y-axis: log 10(GI₅₀) value of Compound 1; x-axis: IRF4 protein expression level as measured by RPPA.

FIG. 6 shows that the sensitivity to Compound 1 correlates with activation of mTORC1 and mTORC2 in a subgroup of DLBCL cell lines as measured via biomarker expression (p-mTOR S2448, p-p70S6K T389, pGSK3b S9 and S21, pAKT 5473 and T308, pTSC2 T1462, pS6 S240/S244 and S235/S236) using RPPA. The level of each biomarker in each DLBCL line is shown in a heatmap (dark gray: high; light gray: low). GI₅₀ values of Compound 1 are shown at the top of the heatmap (light gray: low; dark gray: high).

### 5. DETAILED DESCRIPTION

### 5.1 Definitions

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. Unless otherwise indicated, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aryloxyamine; aralkoxyamine; N-oxide; hydrazine; hydrazide; hydrazone; azide; isocyanate; isothiocyanate; cyanate; thiocyanate; oxygen (═O); B(OH)2, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl and the like. The double bond of an alkenyl group can be unconjugated or conjugated to another unsaturated group. An alkenyl group can be unsubstituted or substituted.

A “cycloalkyl” group is a saturated, or partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclylalkyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amine” group is a radical of the formula: —NH₂.

A “hydroxyl amine” group is a radical of the formula: —N(R^{#})OH or —NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aryloxyamine” group is a radical of the formula: —N(R^{#})O-aryl or —NHO-aryl, wherein R^{#} is as defined above

An “aralkoxyamine” group is a radical of the formula: —N(R^{#})O-aralkyl or —NHO-aralkyl, wherein R^{#} is as defined above.

An “alkylamine” group is a radical of the formula: —NH-alkyl or —N(alkyl), wherein each alkyl is independently as defined above.

An “aminocarbonyl” group is a radical of the formula: —C(═O)N(R^{#})₂, —C(═O)NH(R^{#}) or —C(═O)NH₂, wherein each R^{#} is as defined above.

An “acylamino” group is a radical of the formula: —NHC(═O)(R^{#}) or —N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O—.

A “carboxy” group is a radical of the formula: —C(═O)OH.

A “ketone” group is a radical of the formula: —C(═O)(R^{#}), wherein R^{#} is as defined above.

An “aldehyde” group is a radical of the formula: —CH(═O).

An “ester” group is a radical of the formula: —C(═O)O(R^{#}) or —OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or —NHC(═O)NH₂^{#}, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R#)C(═O)(R^{#}) or —N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{/4}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

A “enamine” group is a radical of the formula: —N(R^{#})C(R^{#})═C(R^{#})₂, —NHC(R^{#})═C(R^{#})₂, —C(N(R^{#)}₂)═C(R^{#})₂, —C(NH(R^{#}))═C(R^{#})₂, —C(NH₂)═C(R^{#)}₂, —C(R^{#})═C(R^{#})(N(R^{#})₂), —C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#)}₂)(R^{#})₂, —C(═N—NH(R^{#}))(R^{#})₂, —C(═N—NH₂)(R^{#})₂, —N(R^{#})(N═C(R^{#)}₂), or —NH(N═C(R^{#)}₂), wherein each R^{#} is independently as defined above.

An “azide” group is a radical of the formula: —N₃.

An “isocyanate” group is a radical of the formula: —N═C═O.

An “isothiocyanate” group is a radical of the formula: —N═C═S.

A “cyanate” group is a radical of the formula: —OCN.

A “thiocyanate” group is a radical of the formula: —SCN.

A “thioether” group is a radical of the formula; —S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R^{#} is as defined above.

A “sulfone” group is a radical of the formula: —S(═O)₂(R^{#}), wherein R^{#} is as defined above.

A “sulfonylamino” group is a radical of the formula: —NHSO₂(R^{#}) or —N(alkyl)SO₂(R^{#}), wherein each alkyl and R^{#} are defined above.

A “sulfonamide” group is a radical of the formula: —S(═O)₂N(R^{#})₂, or —S(═O)₂NH(R^{#}), or —S(═O)₂NH₂, wherein each R^{#} is independently as defined above.

A “phosphonate” group is a radical of the formula: —P(═O)(O(R^{#})₂, —P(═O)(OH)₂, —OP(═O)(O(R^{#})(R^{#}), or —OP(═O)(OH)(R^{#}), wherein each R^{#} is independently as defined above.

A “phosphine” group is a radical of the formula: —P(R^{#})₂, wherein each R^{#} is independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aryloxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the TOR kinase inhibitors include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “clathrate” means a TOR kinase inhibitor, or a salt thereof, in the form of a crystal lattice that contains spaces (e.g., channels) that have a guest molecule (e.g., a solvent or water) trapped within or a crystal lattice wherein a TOR kinase inhibitor is a guest molecule.

As used herein and unless otherwise indicated, the term “solvate” means a TOR kinase inhibitor, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a TOR kinase inhibitor, or a salt thereof, that further includes a stoichiometric or non-stoichiometric amount of water bound by non-covalent intermolecular forces.

As used herein and unless otherwise indicated, the term “prodrug” means a TOR kinase inhibitor derivative that can hydrolyze, oxidize, or otherwise react under biological conditions (in vitro or in vivo) to provide an active compound, particularly a TOR kinase inhibitor. Examples of prodrugs include, but are not limited to, derivatives and metabolites of a TOR kinase inhibitor that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a TOR kinase inhibitor that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The TOR kinase inhibitors can have chiral centers and can occur as racemates, individual enantiomers or diastereomers, and mixtures thereof. All such isomeric forms are included within the embodiments disclosed herein, including mixtures thereof. The use of stereomerically pure forms of such TOR kinase inhibitors, as well as the use of mixtures of those forms are encompassed by the embodiments disclosed herein. For example, mixtures comprising equal or unequal amountsv of the enantiomers of a particular TOR kinase inhibitor may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the TOR kinase inhibitors can include E and Z isomers, or a mixture thereof, and cis and trans isomers or a mixture thereof. In certain embodiments, the TOR kinase inhibitors are isolated as either the cis or trans isomer. In other embodiments, the TOR kinase inhibitors are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

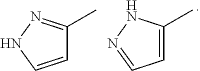

As readily understood by one skilled in the art, a wide variety of functional groups and other stuctures may exhibit tautomerism and all tautomers of the TOR kinase inhibitors are within the scope of the present invention.

It should also be noted the TOR kinase inhibitors can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer and inflammation therapeutic agents, research reagents, e.g., binding assay reagents, and diagnostic agents, e.g., in vivo imaging agents. All isotopic variations of the TOR kinase inhibitors as described herein, whether radioactive or not, are intended to be encompassed within the scope of the embodiments provided herein. In some embodiments, there are provided isotopologues of the TOR kinase inhibitors, for example, the isotopologues are deuterium, carbon-13, or nitrogen-15 enriched TOR kinase inhibitors.

“Treating” as used herein, means an alleviation, in whole or in part, of symptoms associated with a disorder or disease (e.g., cancer or a tumor syndrome), or slowing, or halting of further progression or worsening of those symptoms.

“Preventing” as used herein, means the prevention of the onset, recurrence or spread, in whole or in part, of the disease or disorder (e.g., cancer), or a symptom thereof.

The term “effective amount” in connection with an TOR kinase means an amount alone or in combination capable of alleviating, in whole or in part, a symptom associated with a cancer, or slowing or halting further progression or worsening of those symptoms, or treating or preventing a cancer in a subject having or at risk for having a cancer. The effective amount of the TOR kinase inhibitor, for example in a pharmaceutical composition, may be at a level that will exercise the desired effect; for example, about 0.005 mg/kg of a subject's body weight to about 100 mg/kg of a patient's body weight in unit dosage for both oral and parenteral administration.

The term “cancer” refers to any of various malignant neoplasms characterized by the proliferation of cells that can invade surrounding tissue and metastasize to new body sites. Both benign and malignant tumors are classified according to the type of tissue in which they are found. For example, fibromas are neoplasms of fibrous connective tissue, and melanomas are abnormal growths of pigment (melanin) cells. Malignant tumors originating from epithelial tissue, e.g., in skin, bronchi, and stomach, are termed carcinomas. Malignancies of epithelial glandular tissue such as are found in the breast, prostate, and colon, are known as adenocarcinomas. Malignant growths of connective tissue, e.g., muscle, cartilage, lymph tissue, and bone, are called sarcomas. Lymphomas and leukemias are malignancies arising among white blood cells. Through the process of metastasis, tumor cell migration to other areas of the body establishes neoplasms in areas away from the site of initial appearance. Bone tissues are one of the most favored sites of metastases of malignant tumors, occurring in about 30% of all cancer cases. Among malignant tumors, cancers of the lung, breast, prostate or the like are particularly known to be likely to metastasize to bone.

In the context of neoplasm, cancer, tumor growth or tumor cell growth, inhibition may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention. In this context, the term “prevention” includes either preventing the onset of clinically evident neoplasia altogether or preventing the onset of a preclinically evident stage of neoplasia in individuals at risk. Also intended to be encompassed by this definition is the prevention of transformation into malignant cells or to arrest or reverse the progression of premalignant cells to malignant cells. This includes prophylactic treatment of those at risk of developing the neoplasia.

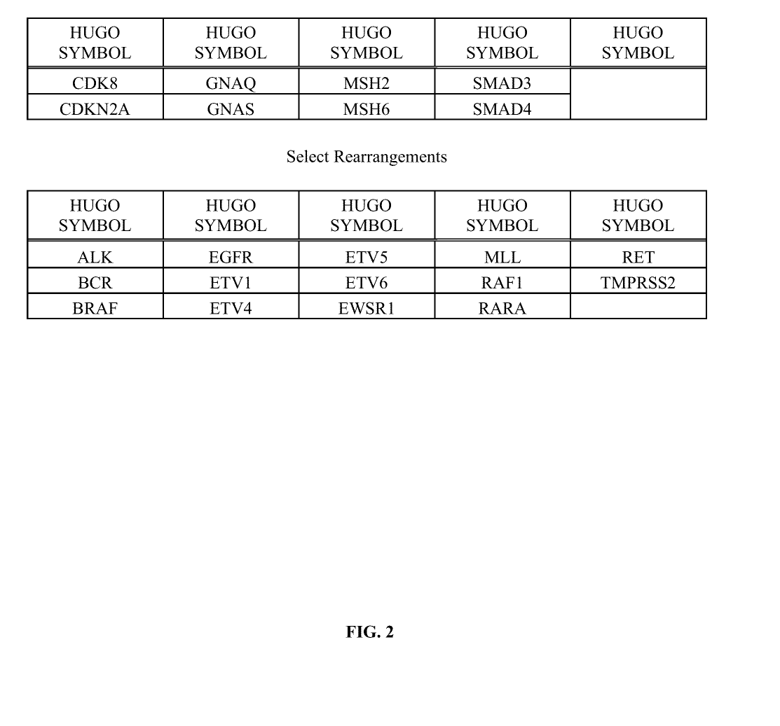

As used herein “wild type” refers to the typical or most common form of a characteristic (for example, gene sequence or presence, or protein sequence, presence, level or activity), as it occurs in nature, and the reference against which all others are compared. As will be understood by one skilled in the art, when used herein, wild type refers to the typical gene sequence(s) or gene expression levels as they most commonly occur in nature. Similarly, a “control patient”, as used herein, is a patient who exhibits the wild type gene sequence(s) or gene or protein expression levels. In certain embodiments, the gene sequence is the gene sequence of one or more of the genes set forth in Table 1, i.e, PIK3CA, RICTOR, TP53, IGF1R and/or PTEN. In one embodiment, the gene sequence is the gene sequence of one or more of RICTOR, TP53 or IGF1R. In some such embodiments, a further gene sequence is PIK3CA. In one embodiment, the gene sequence is the gene sequence of AKT1. In one embodiment, the gene sequence is the gene sequence of AKT2. In certain embodiments, the gene sequence is the gene sequence of one or more of the genes set forth in FIG. 2. In another embodiment, the gene sequence is the gene sequence of one or more of genes set forth in Table 2 or Table 3. In yet another, the gene sequence is the gene sequence of one or more genes set forth in Table 4.

For genetic analysis, tumor samples are collected and DNA is extracted from tumor samples (for example, pre-treatment tumor samples) and submitted for next generation sequencing (for example at Foundation Medicine, Inc (FMI)). A gene is considered to be mutant (variant) if it shows one of the following: mutation(s) (likely or known somatic variants, or variants of unknown significance), or structural variation (deletion, amplification or rearrangement). A gene is considered to be wild type when no sequencing alterations (variants) are detected for this gene. A gene cluster is considered to be mutated if any gene in the cluster is mutated as defined above; otherwise the gene cluster is considered to be wild type.

As used herein, “gene mutation” and “gene variant” indicates a deviation from wild type or non-mutated state. These include single or multiple base changes, nucleotide insertions or nucleotide deletions (single or multiple bases in either case), copy number changes including loss of one copy or focal or large amplifications of segments of DNA, or rearrangements of the DNA, where the strands break and are rejoined in new ways different from the wild type. Additionally, as used herein “gene mutation” refers to a gene mutation resulting in, for example, an increase or a decrease in mRNA expression, an increase or decrease in protein production, a non-functional protein or a protein with altered function, as compared to wild type. As used herein “gene or protein loss” refers to a reduced level of gene or protein or the absence of gene or protein, as compared to wild type levels.

The term “expression” as used herein refers to the transcription from a gene to give an RNA nucleic acid molecule complementary at least in part to a region of one of the two nucleic acid strands of the gene. The term “expression” as used herein also refers to the translation from the RNA molecule to give a protein, a polypeptide or a portion thereof.

The expression of a gene that is “upregulated” is generally “increased” relative to wild type. The expression of a gene that is “downregulated” is generally “decreased” relative to wild type. In certain embodiments, a gene from a patient sample can be “upregulated,” i.e., gene expression can be increased, for example, by about 5%, 10%, 20%, 30%, 40%, 50%, 60%, 70%, 90%, 100%, 200%, 300%, 500%, 1,000%, 5,000% or more of a comparative control, such as wild type. In other embodiments, a gene from a patient sample can be “downregulated,” i.e., gene expression can be decreased, for example, by about 99%, 95%, 90%, 80%, 70%, 60%, 50%, 40%, 30%, 20%, 10%, 1% or less of a comparative control, such as wild type.

As used herein “reduced level” or “loss” means a reduction in level relative to levels observed in wild type. In one embodiment the reduction is 10%-50% or 50%-100%. In some embodiments, the reduction is 20%, 30%, 40%, 50%, 60%, 70%, 80%. 90% or 100% (complete loss) relative to wild type.

The terms “patient” and “subject” as used herein include an animal, including, but not limited to, an animal such as a cow, monkey, horse, sheep, pig, chicken, turkey, quail, cat, dog, mouse, rat, rabbit or guinea pig, in one embodiment a mammal, in another embodiment a human.

In one embodiment, a “patient” or “subject” is a human whose DNA comprises a gene mutation or variant, relative to that of a control patient or wild type. In another embodiment, a “patient” or “subject” is a human whose DNA contains a gene mutation or variant, relative to that of a control patient or wild type. In another embodiment, a “patient” or “subject” is a human having a gene mutation or variant, relative to that of a control patient or wild type. In another embodiment, a “patient” or “subject” is a human having a gene mutation or variant, relative to that of a control patient or wild type, and also having a cancer characterized by a gene mutation or variant, for example breast cancer, DLBCL, GBM, HCC, MM, NET, or NSCLC. In particular embodiments, the gene mutation or variant is identified by certain gene sequence(s), determined using, for example, Sanger sequencing, di-deoxy chain termination sequencing, massively parallel next generation sequencing (NGS), or PCR based methods and compared to wild type, using analytical pipelines that process raw sequence data for tumor samples and reference samples, filter out data artifacts from the sequencing process; filter out known polymorphisms and identify the mutation variants present in the tumor sample (see J. Ross and M. Cronin, Am. J. Clin. Pathol, 136; 527-539 (2011)). In certain embodiments, the mutation is in one or more of the genes set forth in Table 1, i.e. PIK3CA, RICTOR, TP53, IGF1R and/or PTEN. In one embodiment, the mutation is in one or more of RICTOR, TP53 or IGF1R. In some such embodiments, a further mutation is a mutation in PIK3CA. In one embodiment, the mutation is a mutation in the gene sequence of AKT1. In one embodiment, the mutation is a gene amplication mutation in the gene sequence of AKT2. In certain embodiments, the variant is in one or more of the genes set forth in FIG. 2. In certain embodiments, the variant is in one or more of the genes set forth in Table 2 or Table 3. In certain embodiments, the variant is in one or more of the genes set forth in Table 4. In one embodiment, the variant is one or more known somatic-variants, likely-somatic variants, rearrangements, variants-of-unknown-significance, or copy-number variants, for example, amplifications or deletions, or a combination thereof. In one embodiment, the variant is one or more known somatic variants. In another embodiment, the variant is one or more likely somatic-variants. In one embodiment, the variant is one or more rearrangements. In one embodiment, the variant is one or more variants-of-unknown-significance. In one embodiment, the variant is one or more amplifications. In another embodiment, the variant is one or more deletions.

The term “likelihood” generally refers to an increase in the probability of an event. The term “likelihood” when used in reference to the effectiveness of a patient response generally contemplates an increased probability that a cancer or tumor syndrome, or symptom thereof, will be lessened or decreased.

The term “predict” generally means to determine or tell in advance. When used to “predict” the effectiveness of a cancer, for example, the term “predict” can mean that the likelihood of the outcome of the treatment can be determined at the outset, before the treatment has begun, or before the treatment period has progressed substantially.

The terms “determining”, “measuring”, “evaluating”, “assessing” and “assaying” as used herein generally refer to any form of measurement, and include determining if an element is present or not. These terms include both quantitative and/or qualitative determinations.

In the context of a cancer, inhibition may be assessed by inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from randomization (for example, first dose date) until death from any cause, and is measured in the intent-to-treat population. TTP as used herein means the time from randomization (for example, first dose date) until objective tumor progression; TTP does not include deaths. As used herein, PFS means the time from randomization (for example, first dose date) until objective tumor progression or death. In one embodiment, PFS rates will be computed using the Kaplan-Meier estimates. In the extreme, complete inhibition, is referred to herein as prevention or chemoprevention. In this context, the term “prevention” includes either preventing the onset of clinically evident advanced cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

In certain embodiments, the treatment of a cancer may be assessed by Response Evaluation Criteria in Solid Tumors (RECIST 1.1) (see Thereasse P., et al. New Guidelines to Evaluate the Response to Treatment in Solid Tumors. J. of the National Cancer Institute; 2000; (92) 205-216 and Eisenhauer E. A., Therasse P., Bogaerts J., et al. New response evaluation criteria in solid tumours: Revised RECIST guideline (version 1.1). European J. Cancer; 2009; (45) 228-247). Overall responses for all possible combinations of tumor responses in target and non-target lesions with our without the appearance of new lesions are as follows:

| Target lesions | Non-target lesions | New lesions | Overall response |
|---|---|---|---|
| CR | CR | No | CR |
| CR | Incomplete | No | PR |
| | response/SD | | |
| PR | Non-PD | No | PR |
| SD | Non-PD | No | SD |
| PD | Any | Yes or no | PD |
| Any | PD | Yes or no | PD |
| Any | Any | Yes | PD |

CR = complete response;
PR = partial response;
SD = stable disease; and
PD = progressive disease.

With respect to the evaluation of target lesions, complete response (CR) is the disappearance of all target lesions, partial response (PR) is at least a 30% decrease in the sum of the longest diameter of target lesions, taking as reference the baseline sum longest diameter, progressive disease (PD) is at least a 20% increase in the sum of the longest diameter of target lesions, taking as reference the smallest sum longest diameter recorded since the treatment started or the appearance of one or more new lesions and stable disease (SD) is neither sufficient shrinkage to qualify for partial response nor sufficient increase to qualify for progressive disease, taking as reference the smallest sum longest diameter since the treatment started.

With respect to the evaluation of non-target lesions, complete response (CR) is the disappearance of all non-target lesions and normalization of tumor marker level; incomplete response/stable disease (SD) is the persistence of one or more non-target lesion(s) and/or the maintenance of tumor marker level above the normal limits, and progressive disease (PD) is the appearance of one or more new lesions and/or unequivocal progression of existing non-target lesions.

In certain embodiments, the treatment of lymphoma may be assessed by the International Workshop Criteria (IWC) for non-Hodgkin lymphoma (NHL) (see Cheson B D, Pfistner B, Juweid, M E, et. al. Revised Response Criteria for Malignant Lymphoma. J. Clin. Oncol: 2007: (25) 579-586), using the response and endpoint definitions shown below:

| Response | Definition | Nodal Masses | Spleen, liver | Bone Marrow |
|---|---|---|---|---|
| CR | Disappearance | (a) FDG-avid or PET | Not palpable, | Infiltrate cleared on |
| | of all evidence | positive prior to therapy; | nodules | repeat biopsy; if |
| | of disease | mass of any size permitted | disappeared | indeterminate by |
| | | if PET negative | | morphology, |
| | | (b) Variably FDG-avid or | | immunohistochemistry |
| | | PET negative; regression to | | should be negative |
| | | normal size on CT | | |
| PR | Regression of | ≧50% decrease in SPD of | ≧50% | Irrelevant if positive |
| | measurable | up to 6 largest dominant | decrease in | prior to therapy; cell |
| | disease and no | masses; no increase in size | SPD of | type should be specified |
| | new sites | of other nodes | nodules (for | |
| | | (a) FDG-avid or PET | single nodule | |
| | | positive prior to therapy; | in greatest | |
| | | one or more PET positive at | transverse | |
| | | previously involved site | diameter); no | |
| | | (b) Variably FDG-avid or | increase in | |
| | | PET negative; regression on | size of liver or | |
| | | CT | spleen | |
| SD | Failure to | (a) FDG-avid or PET | | |
| | attain CR/PR | positive prior to therapy; | | |
| | or PD | PET positive at prior sites | | |
| | | of disease and no new sites | | |
| | | on CT or PET | | |
| | | (b) Variably FDG-avid or | | |
| | | PET negative; no change in | | |
| | | size of previous lesions on | | |
| | | CT | | |
| PD or | Any new | Appearance of a new | ≧50% increase | New or recurrent |
| relapsed | lesion or | lesion(s) ≧1.5 cm in any | from nadir in | involvement |
| disease | increase by | axis, ≧50% increase in SPD | the SPD of | |
| | ≧50% of | of more than one node, | any previous | |
| | previously | or ≧50% increase in longest | lesions | |
| | involved sites | diameter of a | | |
| | from nadir | previously identifed node | | |
| | | ≧1 cm in short axis | | |
| | | Lesions PET positive if | | |
| | | FDG-avid lymphoma or | | |
| | | PET positive prior to | | |
| | | therapy | | |

Abbreviations:
CR, complete remission;
FDG, [¹⁸F] fluorodeoxyglucose;
PET, positron emission tomography;
CT, computed tomography;
PR, partial remission;
SPD, sum of the product of the diameters;
SD, stable disease;
PD, progressive disease.

| End point | Patients | Definition | Measured from |
|---|---|---|---|
| Primary | | | |
| Overall survival | All | Death as a result of any cause | Entry onto study |
| Progression-free | All | Disease progression or death as a result of | Entry onto study |
| survival | | any cause | |
| Secondary | | | |
| Event-free survival | All | Failure of treatment or death as result of any | Entry onto study |
| | | cause | |
| Time to | All | Time to progression or death as a result of | Entry onto study |
| progression | | lymphoma | |
| Disease-free | In CR | Time to relapse or death as a result of | Documentation of |
| survival | | lymphoma or acute toxicity of treatment | response |
| Response duration | In CR | Time to relapse or progression | Documentation of |
| | or PR | | response |
| Lymphoma- | All | Time to death as a result of lymphoma | Entry onto study |
| specific survival | | | |
| Time to next | All | Time to new treatment | End of primary |
| treatment | | | treatment |

Abbreviations:
CR: complete remission;
PR: partial remission.

In one embodiment, the end point for lymphoma is evidence of clinical benefit. Clinical benefit may reflect improvement in quality of life, or reduction in patient symptoms, transfusion requirements, frequent infections, or other parameters. Time to reappearance or progression of lymphoma-related symptoms can also be used in this end point.

In certain embodiments, the treatment of CLL may be assessed by the International Workshop Guidelines for CLL (see Hallek M, Cheson B D, Catovsky D, et al. Guidelines for the diagnosis and treatment of chronic lymphocytic leukemia: a report from the International Workshop on Chronic Lymphocytic Leukemia updating the National Cancer Institute-Working Group 1996 guidelines. Blood, 2008; (111) 12: 5446-5456) using the response and endpoint definitions shown therein and in particular:

| Parameter | CR | PR | PD |
|---|---|---|---|
| Group A | | | |
| Lymphadenopathy† | None >1.5 cm | Decrease ≧50% | Increase ≧50% |
| Hepatomegaly | None | Decrease ≧50% | Increase ≧50% |
| Splenomegaly | None | Decrease ≧50% | Increase ≧50% |
| Blood lymphocytes | <4000/μL | Decrease ≧50% from | Increase ≧50% over |
| | | baseline | baseline |
| Marrow‡ | Normocellular, <30% | 50% reduction in | |
| | lymphocytes, no B-lymphoid | marrow infiltrate, or | |
| | nodules. Hypocellular | B-lymphoid nodules | |
| | marrow defines CRi (5.1.6). | | |
| Group B | | | |
| Platelet count | >100 000/μL | >100 000/μL or | Decrease of ≧50% |
| | | increase ≧50% over | from baseline |
| | | baseline | secondary to CLL |
| Hemoglobin | >11.0 g/dL | >11 g/dL or increase | Decrease of >2 g/dL |
| | | ≧50% over baseline | from baseline |
| | | | secondary to CLL |
| Neutrophils‡ | >1500/μL | >1500/μL or >50% | |
| | | improvement over | |
| | | baseline | |

Group A criteria define the tumor load;
Group B criteria define the function of the hematopoietic system (or marrow).
CR (complete remission): all of the criteria have to be met, and patients have to lack disease-related constitutional symptoms;
PR (partial remission): at least two of the criteria of group A plus one of the criteria of group B have to be met;
SD is absence of progressive disease (PD) and failure to achieve at least a PR;
PD: at least one of the above criteria of group A or group B has to be met. Sum of the products of multiple lymph nodes (as evaluated by CT scans in clinical trials, or by physical examination in general practice). These parameters are irrelevant for some response categories.

In certain embodiments, the treatment of multiple myeloma may be assessed by the International Uniform Response Criteria for Multiple Myeloma (IURC) (see Durie B G M, Harousseau J-L, Miguel J S, et al. International uniform response criteria for multiple myeloma. Leukemia, 2006; (10) 10: 1-7), using the response and endpoint definitions shown below:

| Response Subcategory | Response Criteria^{a} |
|---|---|
| sCR | CR as defined below plus |
| | Normal FLC ratio and |
| | Absence of clonal cells in bone marrow^{b} by |
| | immunohistochemistry or |
| | immunofluorescence^{c} |
| CR | Negative immunofixation on the serum and urine and |
| | Disappearance of any soft tissue plasmacytomas and |
| | <5% plasma cells in bone marrow^{b} |
| VGPR | Serum and urine M-protein detectable by immunofixation |
| | but not on electrophoresis or 90% or greater reduction in |
| | serum M-protein plus urine M-protein level <100 mg per |
| | 24 h |
| PR | ≧50% reduction of serum M-protein and reduction in 24-h |
| | urinary M-protein by ≧90% or to <200 mg per 24 h |
| | If the serum and urine M-protein are unmeasurable,^{d} a |
| | ≧50% decrease in the difference between involved and |
| | uninvolved FLC levels is required in place of the M- |
| | protein criteria |
| | If serum and urine M-protein are unmeasurable, and serum |
| | free light assay is also unmeasurable, ≧50% reduction in |
| | plasma cells is required in place of M-protein, provided |
| | baseline bone marrow plasma cell percentage was ≧30% |
| | In addition to the above listed criteria, if present at |
| | baseline, a ≧50% reduction in the size of soft tissue |
| | plasmacytomas is also required |
| SD (not recommended for use as an | Not meeting criteria for CR, VGPR, PR or progressive |
| indicator of response; stability of | disease |
| disease is best described by | |
| providing the time to progression | |
| estimates) | |

Abbreviations:
CR, complete response;
FLC, free light chain;
PR, partial response;
SD, stable disease;
sCR, stringent complete response;
VGPR, very good partial response;
^{a}All response categories require two consecutive assessments made at anytime before the institution of any new therapy; all categories also require no known evidence of progressive or new bone lesions if radiographic studies were performed. Radiographic studies are not required to satisfy these response requirements;
^{b}Confirmation with repeat bone marrow biopsy not needed;
^{c}Presence/absence of clonal cells is based upon the κ/λ ratio. An abnormal κ/λ ratio by immunohistochemistry and/or immunofluorescence requires a minimum of 100 plasma cells for analysis. An abnormal ratio reflecting presence of an abnormal clone is κ/λ of >4:1 or <1:2.
^{d}Measurable disease defined by at least one of the following measurements:
Bone marrow plasma cells ≧30%;
Serum M-protein ≧1 g/dl (≧10 gm/l)[10 g/l];
Urine M-protein ≧200 mg/24 h;
Serum FLC assay:
Involved FLC level ≧10 mg/dl (≧100 mg/l);
provided serum FLC ratio is abnormal.

The procedures, conventions, and definitions described below provide guidance for implementing the recommendations from the Response Assessment for Neuro-Oncology (RANO) Working Group regarding response criteria for high-grade gliomas (Wen P., Macdonald, D R., Reardon, D A., et al. Updated response assessment criteria for highgrade gliomas: Response assessment in neuro-oncology working group. J Clin Oncol 2010; 28: 1963-1972). Primary modifications to the RANO criteria for Criteria for Time Point Responses (TPR) can include the addition of operational conventions for defining changes in glucocorticoid dose, and the removal of subjects' clinical deterioration component to focus on objective radiologic assessments. The baseline MRI scan is defined as the assessment performed at the end of the post-surgery rest period, prior to re-initiating compound treatment. The baseline MRI is used as the reference for assessing complete response (CR) and partial response (PR). Whereas, the smallest SPD (sum of the products of perpendicular diameters) obtained either at baseline or at subsequent assessments will be designated the nadir assessment and utilized as the reference for determining progression. For the 5 days preceding any protocol-defined MRI scan, subjects receive either no glucocorticoids or are on a stable dose of glucocorticoids. A stable dose is defined as the same daily dose for the 5 consecutive days preceding the MRI scan. If the prescribed glucocorticoid dose is changed in the 5 days before the baseline scan, a new baseline scan is required with glucocorticoid use meeting the criteria described above. The following definitions will be used.

Measurable Lesions: Measurable lesions are contrast-enhancing lesions that can be measured bidimensionally. A measurement is made of the maximal enhancing tumor diameter (also known as the longest diameter, LD). The greatest perpendicular diameter is measured on the same image. The cross hairs of bidimensional measurements should cross and the product of these diameters will be calculated.

Minimal Diameter: T1-weighted image in which the sections are 5 mm with 1 mm skip. The minimal LD of a measurable lesion is set as 5 mm by 5 mm. Larger diameters may be required for inclusion and/or designation as target lesions. After baseline, target lesions that become smaller than the minimum requirement for measurement or become no longer amenable to bidimensional measurement will be recorded at the default value of 5 mm for each diameter below 5 mm. Lesions that disappear will be recorded as 0 mm by 0 mm.

Multicentric Lesions: Lesions that are considered multicentric (as opposed to continuous) are lesions where there is normal intervening brain tissue between the two (or more) lesions. For multicentric lesions that are discrete foci of enhancement, the approach is to separately measure each enhancing lesion that meets the inclusion criteria. If there is no normal brain tissue between two (or more) lesions, they will be considered the same lesion.

Nonmeasurable Lesions: All lesions that do not meet the criteria for measurable disease as defined above will be considered non-measurable lesions, as well as all nonenhancing and other truly nonmeasurable lesions. Nonmeasurable lesions include foci of enhancement that are less than the specified smallest diameter (i.e., less than 5 mm by 5 mm), nonenhancing lesions (e.g., as seen on T1-weighted post-contrast, T2-weighted, or fluid-attenuated inversion recovery (FLAIR) images), hemorrhagic or predominantly cystic or necrotic lesions, and leptomeningeal tumor. Hemorrhagic lesions often have intrinsic T1-weighted hyperintensity that could be misinterpreted as enhancing tumor, and for this reason, the pre-contrast T1-weighted image may be examined to exclude baseline or interval sub-acute hemorrhage.

At baseline, lesions will be classified as follows: Target lesions: Up to 5 measurable lesions can be selected as target lesions with each measuring at least 10 mm by 5 mm, representative of the subject's disease; Non-target lesions: All other lesions, including all nonmeasurable lesions (including mass effects and T2/FLAIR findings) and any measurable lesion not selected as a target lesion. At baseline, target lesions are to be measured as described in the definition for measurable lesions and the SPD of all target lesions is to be determined. The presence of all other lesions is to be documented. At all post-treatment evaluations, the baseline classification of lesions as target and non-target lesions will be maintained and lesions will be documented and described in a consistent fashion over time (e.g., recorded in the same order on source documents and eCRFs). All measurable and nonmeasurable lesions must be assessed using the same technique as at baseline (e.g., subjects should be imaged on the same MRI scanner or at least with the same magnet strength) for the duration of the study to reduce difficulties in interpreting changes. At each evaluation, target lesions will be measured and the SPD calculated. Non-target lesions will be assessed qualitatively and new lesions, if any, will be documented separately. At each evaluation, a time point response will be determined for target lesions, non-target lesions, and new lesion. Tumor progression can be established even if only a subset of lesions is assessed. However, unless progression is observed, objective status (stable disease, PR or CR) can only be determined when all lesions are assessed.

Confirmation assessments for overall time point responses of CR and PR will be performed at the next scheduled assessment, but confirmation may not occur if scans have an interval of <28 days. Best response, incorporating confirmation requirements, will be derived from the series of time points.

In certain embodiments, treatment of a cancer may be assessed by the inhibition of phosphorylation of S6RP, 4E-BP1, AKT and/or DNA-PK in circulating blood and/or tumor cells, and/or skin biopsies or tumor biopsies/aspirates, before, during and/or after treatment with a TOR kinase inhibitor. For example, the inhibition of phosphorylation of S6RP, 4E-BP1, AKT and/or DNA-PK is assessed in B-cells, T-cells and/or monocytes. In other embodiments, treatment of a cancer may be assessed by the inhibition of DNA-dependent protein kinase (DNA-PK) activity in skin samples and/or tumor biopsies/aspirates, such as by assessment of the amount of pDNA-PK S2056 as a biomarker for DNA damage pathways, before, during, and/or after TOR kinase inhibitor treatment. In one embodiment, the skin sample is irradiated by UV light.

In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

### 5.2 Tor Kinase Inhibitors

The compounds provided herein are generally referred to as “TOR kinase inhibitor(s).” In a specific embodiment, the TOR kinase inhibitors do not include rapamycin or rapamycin analogs (rapalogs).

In one embodiment, the TOR kinase inhibitors include compounds having the following formula (I):

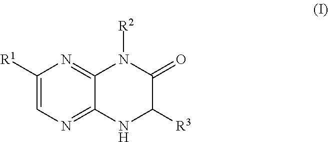

and pharmaceutically acceptable salts, clathrates, solvates, stereoisomers, tautomers, metabolites, isotopologues and prodrugs thereof, wherein:

R¹ is substituted or unsubstituted C₁₋₈ alkyl, substituted or unsubstituted aryl, substituted or unsubstituted cycloalkyl, substituted or unsubstituted heterocyclyl, or substituted or unsubstituted heterocyclylalkyl;

R² is H, substituted or unsubstituted C₁₋₈ alkyl, substituted or unsubstituted cycloalkyl, substituted or unsubstituted heterocyclyl, substituted or unsubstituted heterocyclylalkyl, substituted or unsubstituted aralkyl, or substituted or unsubstituted cycloalkylalkyl;

R³ is H, or a substituted or unsubstituted C₁₋₈ alkyl,

wherein in certain embodiments, the TOR kinase inhibitors do not include 7-(4-hydroxyphenyl)-1-(3-methoxybenzyl)-3,4-dihydropyrazino[2,3-b]pyrazin-2(1H)-one, depicted below:

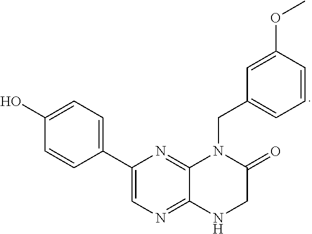 [SMILES: COc1cccc(CN2C(=O)CNc3ncc(-c4ccc(O)cc4)nc32)c1 ; "."]

In some embodiments of compounds of formula (I), R¹ is substituted or unsubstituted aryl or substituted or unsubstituted heteroaryl. For example, R¹ is phenyl, pyridyl, pyrimidyl, benzimidazolyl, 1H-pyrrolo[2,3-b]pyridyl, indazolyl, indolyl, 1H-imidazo[4,5-b]pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl (for example, methyl), substituted or unsubstituted heterocyclyl (for example, a substituted or unsubstituted triazolyl or pyrazolyl), aminocarbonyl, halogen (for example, fluorine), cyano, hydroxyalkyl and hydroxy. In other embodiments, R¹ is pyridyl substituted with one or more substituents independently selected from the group consisting of substituted or unsubstituted C₁₋₈ alkyl (for example, methyl), substituted or unsubstituted heterocyclyl (for example, a substituted or unsubstituted triazolyl), halogen, aminocarbonyl, cyano, hydroxyalkyl (for example, hydroxypropyl), —OR, and —NR₂, wherein each R is independently H, or a substituted or unsubstituted C₁₋₄ alkyl. In some embodiments, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In some embodiments, R¹ is

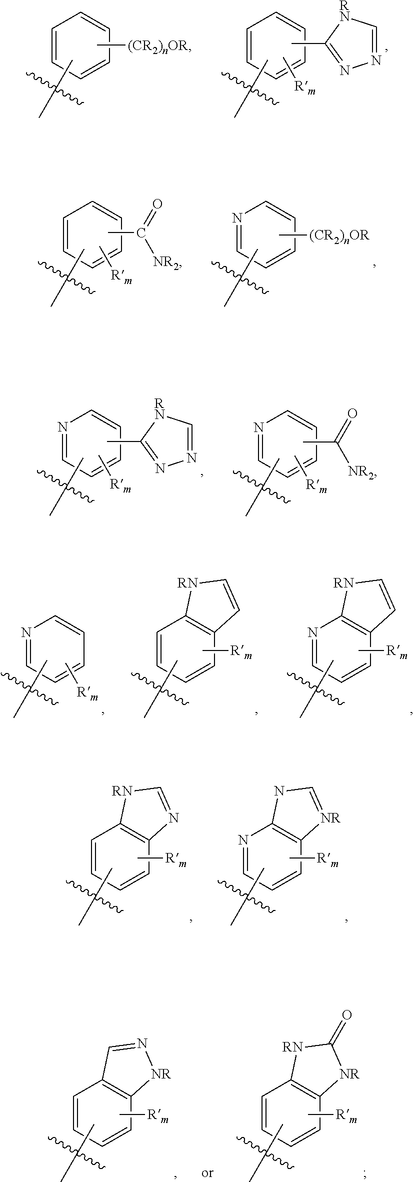

wherein R is at each occurrence independently H, or a substituted or unsubstituted C₁₋₄ alkyl (for example, methyl); R′ is at each occurrence independently a substituted or unsubstituted C₁₋₄ alkyl (for example, methyl), halogen (for example, fluoro), cyano, —OR, or —NR₂; m is 0-3; and n is 0-3. It will be understood by those skilled in the art that any of the substituents R′ may be attached to any suitable atom of any of the rings in the fused ring systems.

In some embodiments of compounds of formula (I), R¹ is

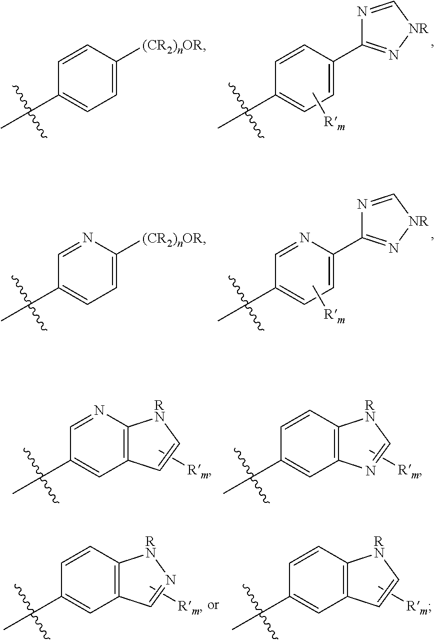

wherein R is at each occurrence independently H, or a substituted or unsubstituted C₁₋₄ alkyl; R′ is at each occurrence independently a substituted or unsubstituted C₁₋₄ alkyl, halogen, cyano, —OR or —NR₂; m is 0-3; and n is 0-3.

In some embodiments of compounds of formula (I), R² is H, substituted or unsubstituted C₁₋₈ alkyl, substituted or unsubstituted cycloalkyl, substituted or unsubstituted heterocyclyl, substituted or unsubstituted C₁₋₄ alkyl-heterocyclyl, substituted or unsubstituted C₁₋₄ alkyl-aryl, or substituted or unsubstituted C₁₋₄ alkyl-cycloalkyl. For example, R² is H, methyl, ethyl, n-propyl, isopropyl, n-butyl, sec-butyl, isobutyl, tert-butyl, n-pentyl, isopentyl, cyclopentyl, cyclohexyl, tetrahydrofuranyl, tetrahydropyranyl, (C₁₋₄ alkyl)-phenyl, (C₁₋₄ alkyl)-cyclopropyl, (C₁₋₄ alkyl)-cyclobutyl, (C₁₋₄ alkyl)-cyclopentyl, (C₁₋₄ alkyl)-cyclohexyl, (C₁₋₄ alkyl)-pyrrolidyl, (C₁₋₄ alkyl)-piperidyl, (C₁₋₄ alkyl)-piperazinyl, (C₁₋₄ alkyl)-morpholinyl, (C₁₋₄ alkyl)-tetrahydrofuranyl, or (C₁₋₄ alkyl)-tetrahydropyranyl, each optionally substituted.

In other embodiments, R² is H, C₁₋₄ alkyl, (C₁₋₄ alkyl)(OR),

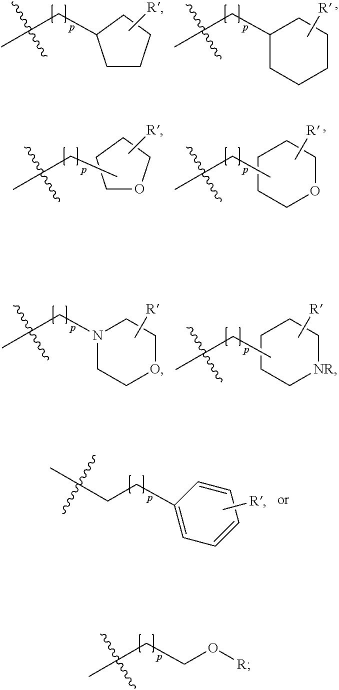

wherein R is at each occurrence independently H, or a substituted or unsubstituted C₁₋₄ alkyl (for example, methyl); R′ is at each occurrence independently H, —OR, cyano, or a substituted or unsubstituted C₁₋₄ alkyl (for example, methyl); and p is 0-3.

In other embodiments of compounds of formula (I), R² is H, C₁₋₄ alkyl, (C₁₋₄alkyl)(OR),

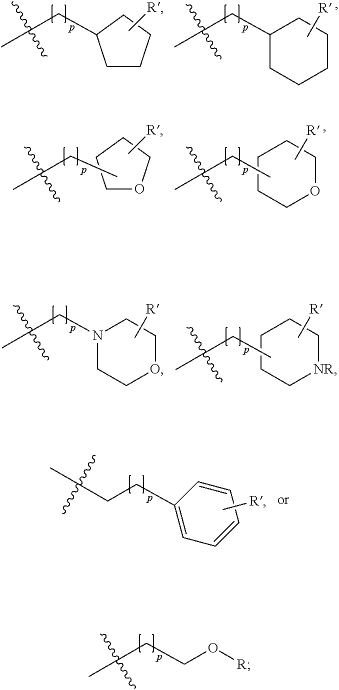

wherein R is at each occurrence independently H, or a substituted or unsubstituted C₁₋₂ alkyl; R′ is at each occurrence independently H, —OR, cyano, or a substituted or unsubstituted C₁₋₂ alkyl; and p is 0-1.

In other embodiments of compounds of formula (I), R³ is H.

In some such embodiments described herein, R¹ is substituted or unsubstituted aryl, or substituted or unsubstituted heteroaryl. For example, R¹ is phenyl, pyridyl, pyrimidyl, benzimidazolyl, 1H-pyrrolo[2,3-b]pyridyl, indazolyl, indolyl, 1H-imidazo[4,5-b]pyridine, pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl, substituted or unsubstituted heterocyclyl, aminocarbonyl, halogen, cyano, hydroxyalkyl and hydroxy. In others, R¹ is pyridyl substituted with one or more substituents independently selected from the group consisting of C₁₋₈ alkyl, substituted or unsubstituted heterocyclyl, halogen, aminocarbonyl, cyano, hydroxyalkyl, —OR, and —NR₂, wherein each R is independently H, or a substituted or unsubstituted C₁₋₄ alkyl. In still others, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In one embodiment, R¹ is pyridyl substituted with one or more substituents independently selected from the group consisting of C₁₋₈ alkyl, substituted or unsubstituted heterocyclyl, halogen, aminocarbonyl, cyano, hydroxyalkyl, —OR, and —NR₂, wherein each R is independently H, or a substituted or unsubstituted C₁₋₄ alkyl, and R² is H, substituted or unsubstituted C₁₋₈ alkyl, substituted or unsubstituted cycloalkyl, substituted or unsubstituted heterocyclyl, substituted or unsubstituted C₁₋₄ alkyl-heterocyclyl, substituted or unsubstituted C₁₋₄ alkyl-aryl, or substituted or unsubstituted C₁₋₄ alkyl-cycloalkyl. In some such embodiments, R¹ is pyridyl substituted with one or more substituents independently selected from the group consisting of C₁₋₈ alkyl, substituted or unsubstituted heterocyclyl, or hydroxyalkyl, and R² is substituted or unsubstituted C₁₋₈ alkyl, or substituted or unsubstituted cycloalkyl.

In certain embodiments, the compounds of formula (I) have an R¹ group set forth herein and an R² group set forth herein.

In some embodiments of compounds of formula (I), the compound at a concentration of 10 μM inhibits mTOR, DNA-PK, PI3K, or a combination thereof by at least about 50%. Compounds of formula (I) may be shown to be inhibitors of the kinases above in any suitable assay system.

Representative TOR kinase inhibitors of formula (I) include compounds from Table A.

TABLE A
7-(5-fluoro-2-methyl-4-(1H-1,2,4-triazol-3-yl)phenyl)-1-((trans-4-methoxycyclohexyl)methyl)-
3,4-dihydropyrazino[2,3-b]pyrazin-2(1H)-one;
7-(6-(1H-1,2,4-triazol-3-yl)pyridin-3-yl)-1-(cis-4-methoxycyclohexyl)-3,4-dihydropyrazino[2,3
b]pyrazin-2(1H)-one;
7-(1H-pyrrolo[2,3-b]pyridin-3-yl)-1-1(2-(tetrahydro-2H-pyran-4-yl)ethyl)-3,4-
dihydropyrazino[2,3-b]pyrazin-2(1H)-one;
7-(5-fluoro-2-methyl-4-(1H-1,2,4-triazol-3-yl)phenyl)-1-((cis-4-methoxycyclohexyl)methyl)-3,4-
dihydropyrazino[2,3-b]pyrazin-2(1H)-one;
1-ethyl-7-(1H-pyrrolo [3,2-b]pyridin-5-yl)-3,4-dihydropyrazino[2,3-b]pyrazin-2(1H)-one;
7-(6-(1H-1,2,4-triazol-3-yl)pyridin-3-yl)-1-((cis-4-methoxycyclohexyl)methyl)-3,4-
dihydropyrazino[2,3-b]pyrazin-2(1H)-one;
7-(1H-benzo[d]imidazol-4-yl)-1-(2-(tetrahydro-2H-pyran-4-yl)ethyl)-3,4-dihydropyrazino[2,3-
b]pyrazin-2(1H)-one;
7-(1H-pyrrolo[2,3-b]pyridin-4-yl)-1-(2-(tetahydro-2H-pyran-4-yl)ethyl)-3,4-
dihydropyrazino[2,3-b]pyrazin-2(1H)-one;
7-(6-(1H-1,2,4-triazol-3-yl)pyridin-3-yl)-1-((trans-4-methoxycyclohexyl)methyl)-3,4-
dihydropyrazino[2,3-b]pyrazin-2(1H)-one;
7-(6-(1H-1,2,4-triazol-3-yl)pyridin-3-yl)-1-((trans-4-hydroxycyclohexyl)methyl)-3,4-
dihydropyrazino[2,3-b]pyrazin-2(1H)-one;
7-(6-(1H-1,2,4-triazol-3-yl)pyridin-3-yl)-1-(cis-4-hydroxycyclohexyl)-3,4-dihydropyrazino[2,3-
b]pyrazin-2(1H)-one;
7-(5-fluoro-2-methyl-4-(1H-1,2,4-triazol-3-yl)phenyl)-1-(cis-4-hydroxycyclohexyl)-3,4-
dihydropyrazino[2,3-b]pyrazin-2(1H)-one;
7-(6-(1H-1,2,4-triazol-3-yl)pyridin-3-yl)-1-(tetrahydro-2H-pyran-4-yl)-3,4-dihydropyrazino[2,3-
b]pyrazin-2(1H)-one;
7-(6-(1H-1,2,4-triazol-3-yl)pyridin-3-yl)-1-(2-methoxyethyl)-3,4-dihydropyrazino[2,3-b]pyrazin-
2(1H)-one;
7-(6-(1H-1,2,4-triazol-3-yl)pyridin-3-yl)-1-ethyl-3,4-dihydropyrazino[2,3-b]pyrazin-2(1H)-one;
7-(5-fluoro-2-methyl-4-(1H-1,2,4-triazol-3-yl)phenyl)-1-((cis-4-hydroxycyclohexyl)methyl)-3,4-
dihydropyrazino[2,3-b]pyrazin-2(1H)-one;
7-(5-fluoro-2-methyl-4-(1H-1,2,4-triazol-3-yl)phenyl)-1-(tetrahydro-2H-pyran-4-yl)-3,4-
dihydropyrazino[2,3-b]pyrazin-2(1H)-one;
7-(1H-indol-4-yl)-1-(2-(tetrahydro-2H-pyran-4-yl)ethyl)-3,4-dihydropyrazino
2(1H)-one;
7-(5-fluoro-2-methyl-4-(1H-1,2,4-triazol-3-yl)phenyl)-1-((trans-4-hydroxycyclohexyl)methyl)-
3,4-dihydropyrazino[2,3-b]pyrazin-2(1H)-one;
7-(6-(1H-1,2,4-triazol-3-yl)pyridin-3-yl)-1-((cis-4-hydroxycyclohexyl)methyl)-3,4-
dihydropyrazin[2,3-b]pyrazin-2(1H)-one;\
7-(6-(1H-1,2,4-triazol-3-yl)pyridin-3-yl)-1-(trans-4-hydroxycyclohexyl)-3,4-
dihydropyrazino[2,3-b]pyrazin-2(1H)-one;
7-(6-(1H-1,2,4-triazol-3-yl)pyridin-3-yl)-1-(trans-4-methoxycyclohexyl)-3,4-
dihydropyrazino[2,3-b]pyrazin-2(1H)-one;
7-(6-(1H-1,2,4-triazol-3-yl)pyridin-3-yl)-1-isopropyl-3,4-dihydropyrazino[2,3-b]pyrazin-2(1H)-
one;
7-(5-fluoro-2-methyl-4-(1H-1,2,4-triazol-3-yl)phenyl)-1-(trans-4-methoxycyclohexyl)-3,4-
dihydropyrazino[2,3-b]pyrazin-2(1H)-one;
7-(5-fluoro-2-methyl-4-(1H-1,2,4-triazol-3-yl)phenyl)-1-(trans-4-hydroxycyclohexyl)-3,4-
dihydropyrazino[2,3-b]pyrazin-2(1H)-one;
7-(5-fluoro-2-methyl-44 1H-1,2,4-triazol-3-yl)phenyl)-1-(2-methoxyethyl)-3,4-
dihydropyrazino[2,3-b]pyrazin-2(1H)-one;
7-(5-fluoro-2-methyl-4-(1H-1,2,4-triazol-3-yl)phenyl)-1-isopropyl-3,4-dihydropyrazino[2,3-
b]pyrazin-2(1H)-one;
1-ethyl-7-(5-fluoro-2-methyl-4-(1H-1,2,4-triazol-3-yl)phenyl)-3,4-dihydropyrazino[2,3-
b]pyrazin-2(1H)-one;
7-(2-hydroxypyridin-4-yl)-1-(2-(tetrahydro-2H-pyran-4-yl)ethyl)-3,4-dihydropyrazino[2,3-
b]pyrazin-2(1H)-one;
1-isopropyl-7-(4-methyl-6-(1H-1,2,4-triazol-3-yl)pyridin-3-yl)-3,4-dihydropyrazino[2,3-
b]pyrazin-2(1H)-one;
5-(8-isopropyl-7-oxo-5,6,7,8-tetrahydropyrazino[2,3-b]pyrazin-2-yl)-4-methylpicolinamide;
7-(1H-indazol-4-yl)-1-(2-(tetrahydro-2H-pyran-4-yl)ethyl)-3,4-dihydropyrazino[2,3-b]pyrazin-
2(1H)-one;
7-(2-aminopyrimidin-5-yl)-1-(2-(tetrahydro-2H-pyran-4-yl)ethyl)-3,4-dihydropyrazino[2,3-b]
b]pyrazin-2(1H)-one;
7-(2-aminopyridin-4-yl)-1-(2-(tetrahydro-2H-pyran-4-yl)ethyl)-3,4-dihydropyrazino[2,3-b]
b]pyrazin-2(1H)-one;
7-(6-(methylamino)pyridin-3-yl)-1-(2-(tetrahydro-2H-pyran-4-yl)ethyl)-3,4-dihydropyrazino
[2,3-b]pyrazin-2(1H)-one;
7-(6-hydroxypyridin-3-yl)-1-(2-(tetrahydro-2H-pyran-4-yl)ethyl)-3,4-dihydropyrazino[2,3-
b]pyrazin-2(1H)-one;
7-(4-(1H-pyrazol-3-yl)phenyl)-1-(2-methoxyethyl)-3,4-dihydropyrazino[2,3-b]pyrazin-2(1H)-
one;
7-(pyridin-3-yl)-1-(2-(tetrahydro-2H-pyran-4-yl)ethyl)-3,4-dihydropyrazino[2,3-b]pyrazin-
2(1H)-one;
7-(1H-indazol-4-yl)-1-(2-methoxyethyl)-3,4-dihydropyrazino[2,3-b]pyrazin-2(1H)-one;
7-(1H-indazol-6-yl)-1-(2-methoxyethyl)-3,4-dihydropyrazino[2,3-b]pyrazin-2(1H)-one;
7-(pyrimidin-5-yl)-1-(2-(tetrahydro-2H-pyran-4-yl)ethyl)-3,4-dihydropyrazino[2,3-b]pyrazin-
2(1H)-one;
7-(6-methoxypyridin-3-yl)-1-(2-(tetrahydro-2H-pyran-4-yl)ethyl)-3,4-dihydropyrazino[2,3-
b]pyrazin-2(1H)-one;
1-(2-methoxyethyl)-7-(1H-pyrrolo[2,3-b]pyridin-5-yl)-3,4-dihydropyrazino[2,3-b]pyrazin-2(1H)-
one;
1-ethyl-7-(1H-pyrrolo[2,3-b]pyridin-5-yl)-3,4-dihydropyrazino[2,3-b]pyrazin-2(1H)-one;
1-ethyl-7-(1H-indazol-4-yl)-3,4-dihydropyrazino[2,3-b]pyrazin-2(1H)-one;
7-(pyridin-4-yl)-1-(2-(tetrahydro-2H-pyran-4-yl)ethyl)-3,4-dihydropyrazino[2,3-b]pyrazin-2(1H)-
one;
7-(6-aminopyridin-3-yl)-1-(2-(tetrahydro-2H-pyran-4-yl)ethyl)-3,4-dihydropyrazino[2,3-
b]pyrazin-2(1H)-one;
1-methyl-7-(2-methyl-6-(4H-1,2,4-triazol-3-yl)pyridin-3-yl)-3,4-dihydropyrazino[2,3-b]pyrazin-
2(1H)-one;
2-(2-hydroxypropan-2-yl)-5-(8-(trans-4-methoxycyclohexyl)-7-oxo-5,6,7,8-
tetrahydropyrazino[2,3-b]pyrazin-2-yl)pyridine 1-oxide
4-methyl-5-(7-oxo-8-((tetrahydro-2H-pyran-4-yl)methyl)-5,6,7,8-tetrahydropyrazino[2,3-
b]pyrazin-2-yl)picolinamide;
5-(8-((cis-4-methoxycyclohexyl)methyl)-7-oxo-5,6,7,8-tetrahydropyrazino[2,3-b]pyrazin-2-yl)-4-
methylpicolinamide;
7-(1H-pyrazol-4-yl)-1-(2-(tetrahydro-2H-pyran-4-yl)ethyl)-3,4-dihydropyrazino[2,3-b]pyrazin-
2(1H)-one;
1-(trans-4-methoxycyclohexyl)-7-(4-methyl-6-(1H-1,2,4-triazol-3-yl)pyridin-3-yl)-3,4-
dihydropyrazino[2,3-b]pyrazin-2(1H)-one;
3((7-(2-methyl-6-(4H-1,2,4-triazol-3-yl)pyridin-3-yl)-2-oxo-3,4-dihydropyrazino[2,3-b]pyrazin-
1(2H)-yl)methyl)benzonitrile;
1-((trans-4-methoxycyclohexyl)methyl)-7-(4-methyl-6-(1H-1,2,4-triazol-3-yl)pyridin-3-yl)-3,4-
dihydropyrazino[2,3-b]pyrazin-2(1H)-2(1H)-one;
3-(7-oxo-8-(2-(tetrahydro-2H-pyran-4-yl)ethyl)-5,6,7,8-tetrahydropyrazino[2,3-b]pyrazin-2-
yl)benzamide;
5-(8-((trans-4-methoxycyclohexyl)methyl)-7-oxo-5,6,7,8-tetrahydropyrazino[2,3-b]pyrazin-2-yl)-
4-methylpicolinamide;
3-((7-(6-(2-hydroxypropan-2-yl)pyridin-3-yl)-2-oxo-3,4-dihydropyrazino[2,3-b]pyrazin-1(2H)-
yl)methyl)benzonitrile;
7-(6-(2-hydroxypropan-2-yl)pyridin-3-yl)-1-((1R,3R)-3-methoxycyclopentyl)-3,4-
dihydropyrazino[2,3-b]pyrazin-2(1H)-one;
7-(6-(2-hydroxypropan-2-yl)pyridin-3-yl)-1-((1S,3R)-3-methoxycyclopentyl)-3,4-
dihydropyrazino[2,3-b]pyrazin-2(1H)-one;
7-(6-(2-hydroxypropan-2-yl)pyridin-3-yl)-1-((1S,3S)-3-methoxycyclopentyl)-3,4-
dihydropyrazino[2,3-b]pyrazin-2(1H)-one;
7-(6-(2-hydroxypropan-2-yl)pyridin-3-yl)-1-((1R,3S)-3-methoxycyclopentyl)-3,4-
dihydropyrazino[2,3-b]pyrazin-2(1H)-one;
7-(1H-indazol-6-yl)-1-(2-(tetrahydro-2H-pyran-4-yl)ethyl)-3,4-dihydropyrazino[2,3-b]pyrazin-
2(1H)-one;
7-(2-methyl-6-(4H-1,2,4-triazol-3-yl)pyridin-3-yl)-1-(2-morpholinoethyl)-3,4-
dihydropyrazino[2,3-b]pyrazin-2(1H)-one;
1-(trans-4-hydroxycyclohexyl)-7-(2-methyl-6-(4H-1,2,4-triazol-3-yl)pyridin-3-yl)-3,4-
dihydropyrazino[2,3-b]pyrazin-2(1H)-one;
1-(cis-4-hydroxycyclohexyl)-7-(2-methyl-6-(4H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-(2-morpholinoethyl)-3,4-dihydropyrazino[2,3-
b]pyrazin-2(1H)-one;
1-isopropyl-7-(2-methyl-6-(4H-1,2,4-triazol-3-yl)pyridin-3-yl)-3,4-dihydropyrazino[2,3-
b]pyrazin-2(1H)-one;
7-(1H-imidazo[4,5-b]pyridin-6-yl)-1-(2-(tetrahydro-2H-pyran-4-yl)ethyl)-3,4-
dihydropyrazino[2,3-b]pyrazin-2(1H)-one;
1-((cis-4-methoxycyclohexyl)methyl)-7-(2-methyl-6-(1H-1,2,4-triazol-3-yl)pyridin-3-yl)-3,4-
dihydropyrazino[2,3-b]pyrazin-2(1H)-one;
1-(trans-4-hydroxycyclohexyl)-7-(6-(2-hydroxypropan-2-yl)pyridin-3-yl)-3,4-
dihydropyrazino[2,3-b]pyrazin-2(1H)-one;
1-(cis-4-hydroxycyclohexyl)-7-(6-(2-hydroxypropan-2-yl)pyridin-3-yl)-3,4-dihydropyrazino[2,3-
b]pyrazin-2(1H)-one;
4-(7-oxo-8-(2-(tetrahydro-2H-pyran-4-yl)ethyl)-5,6,7,8-tetrahydropyrazino[2,3-b]pyrazin-2-
yl)benzamide;
7-(1H-indazol-5-yl)-1-(2-(tetrahydro-2H-pyran-4-yl)ethyl)-3,4-dihydropyrazino[2,3-b]pyrazin-
2(1H)-one;
7-(1H-pyrrolo[2,3-b]pyridin-5-yl)-1-(2-(tetrahydro-2H-pyran-4-yl)-3,4-
dihydropyrazino[2,3-b]pyrazin-2(1H)-one;
7-(2-methyl-6-(4H-1,2,4-triazol-3-yl)pyridin-3-yl)-1-(tetrahydro-2H-pyran-4-yl)-3,4-
dihydropyrazino[2,3-b]pyrazin-2(1H)-one;
1-((1S,3R)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1R,3R)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1R,3S)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1S,3S)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
7-(1H-indol-5-yl)-1-(2-(tetrahydro-2H-pyran-4-yl)ethyl)-3,4-dihydropyrazino[2,3-b]pyrazin-
2(1H)-one;
1-ethyl-7-(2-methyl-6-(4H-1,2,4-triazol-3-yl)pyridin-3-yl)-3,4-dihydropyrazino[2,3-b]pyrazin-
2(1H)-one;
7-(1H-indol-6-yl)-1-(2-(tetrahydro-2H-pyran-4-yl)ethyl)-3,4-dihydropyrazino[2,3-b]pyrazin-
2(1H)-one;
7-(4-(2-hydroxypropan-2-yl)phenyl)-1-(trans-4-methoxycyclohexyl)-3,4-dihydropyrazino[2,3-
b]pyrazin-2(1H)-one;
7-(6-(2-hydroxypropan-2-yl)pyridin-3-yl)-1-(tetrahydro-2H-pyran-4-yl)-3,4-dihydropyrazino[2,3-
b]pyrazin-2(1H)-one;
1-((trans-4-methoxycyclohexyl)methyl)-7-(2-methyl-6-(1H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-((cis-4-methoxycyclohexyl)methyl)-3,4-
dihydropyrazino[2,3-b]pyrazin-2(1H)-one;
1-(2-methoxyethyl)-7-(4-methyl-2-(methylamino)-1H-benzo[d]imidazol-6-yl-3,4-
dihydropyrazino[2,3-b]pyrazin-2(1H)-one;
7-(7-methyl-2-oxo-2,3-dihydro-1H-benzo[d]imidazol-5-yl)-1-((tetrahydro-2H-pyran-4-
yl)methyl)-3,4-dihydropyrazino[2,3-b]pyrazin-2(1H)-one;
7-(2-methyl-4-(4H-1,2,4-triazol-3-yl)phenyl)-3,4-dihydropyrazino[2,3-b]pyrazin-2(1H)-one;
1-(2-methoxyethyl)-7-(4-methyl-6-(1H-1,2,4-triazol-3-yl)pyridin-3-yl)-3,4-dihydropyrazino[2,3-
b]pyrazin-2(1H)-one;
1-benzyl-7-(2-methyl-4-(4H-1,2,4-triazol-3-yl)phenyl)-3,4-dihydropyrazino[2,3-b]pyrazin-2(1H)-
one;
7-(3-fluoro-4-(4H-1,2,4-triazol-3-yl)phenyl)-1-(2-methoxyethyl)-3,4-dihydropyrazino[2,3-
b]pyrazin-2(1H)-one;
7-(3-fluoro-4-(4H-1,2,4-triazol-3-yl)phenyl)-1-(2-(tetrahydro-2H-pyran-4-yl)ethyl)-3,4-
dihydropyrazino[2,3-b]pyrazin-2(1H)-one;
7-(3-fluoro-2-methyl-4-(1H-1,2,4-triazol-3-yl)phenyl)-1-(2-methoxyethyl)-3,4-
dihydropyrazino[2,3-b]pyrazin-2(1H)-one;
1-(trans-4-methoxycyclohexyl)-7-(2-methyl-6-(4H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-(trans-4-methoxycyclohexyl)-3,4-
dihydropyrazino[2,3-b]pyrazin-2(1H)-one;
7-(5-fluoro-2-methyl-4-(4H-1,2,4-triazol-3-yl)phenyl)-1-(2-(tetrahydro-2H-pyran-4-yl)ethyl)-3,4-
dihydropyrazino[2,3-b]pyrazin-2(1H)-one;
7-(3-fluoro-2-methyl-4-(1H-1,2,4-triazol-3-yl)phenyl)-1-(2-(tetrahydro-2H-pyran-4-yl)ethyl)-3,4-
dihydropyrazino[2,3-b]pyrazin-2(1H)-one;
1-(2-methoxyethyl)-7-(2-methyl-6-(4H-1,2,4-triazol-3-yl)pyridin-3-yl)-3,4-dihydropyrazino[2,3-
b]pyrazin-2(1H)-one;
7-(6-(2-hydroxypropan-2-yl)pyridin-3-yl)-1-((trans-4-methoxycyclohexyl)methyl)-3,4-
dihydropyrazino[2,3-b]pyrazin-2(1H)-one;
1-(cyclopentylmethyl)-7-(6-(2-hydroxypropan-2-yl)pyridin-3-yl)-3,4-dihydropyrazino[2,3-
b]pyrazin-2(1H)-one;
7-(4-(2-hydroxypropan-2-yl)phenyl)-1-(2-methoxyethyl)-3,4-dihydropyrazino[2,3-b]pyrazin-
2(1H)-one;
(S)-7-(6-(1-hydroxyethyl)pyridin-3-yl)-1-(2-(tetrahydro-2H-pyran-4-yl)ethyl)-3,4-
dihydropyrazino[2,3-b]pyrazin-2(1H)-one;
(R)-7-(6-(1-hydroxyethyl)pyridin-3-yl)-1-(2-(tetrahydro-2H-pyran-4-yl)ethyl)-3,4-
dihydropyrazino[2,3-b]pyrazin-2(1H)-one;
7-(2-methyl-6-(4H-1,2,4-triazol-3-yl)pyridin-3-yl)-1-((tetrahydro-2H-pyran-4-yl)methyl)-3,4-
dihydropyrazino[2,3-b]pyrazin-2(1H)-one;
7-(4-(2-hydroxypropan-2-yl)phenyl)-1-(2-(tetrahydro-2H-pyran-4-yl)ethyl)-3,4-
dihydropyrazino[2,3-b]pyrazin-2(1H)-one;
7-(6-(2-hydroxypropan-2-yl)pyridin-3-yl)-1-(4-(trifluoromethyl)benzyl)-3,4-dihydropyrazino[2,3-
b]pyrazin-2(1H)-one;
7-(6-(2-hydroxypropan-2-yl)pyridin-3-yl)-1-(3-(trifluoromethyl)benzyl)-3,4-dihydropyrazino[2,3-
b]pyrazin-2(1H)-one;
7-(6-(2-hydroxypropan-2-yl)pyridin-3-yl)-1-(3-methoxypropyl)-3,4-dihydropyrazino[2,3-
b]pyrazin-2(1H)-one;
7-(4-methyl-6-(1H-1,2,4-triazol-3-yl)pyridin-3-yl)-1-(2-(tetrahydro-2H-pyran-4-yl)ethyl)-3,4-
dihydropyrazino[2,3-b]pyrazin-2(1H)-one;
7-(6-(2-hydroxypropan-2-yl)pyridin-3-yl)-1-(2-methoxyethyl)-3,4-dihydropyrazino [2,3-
b]pyrazin-2(1H)-one;
7-(6-(2-hydroxypropan-2-yl)pyridin-3-yl)-1-((tetrahydro-2H-pyran-4-yl)methyl)-3,4-
dihydropyrazino[2,3-b]pyrazin-2(1H)-one;
7-(4-methyl-2-(methylamino)-1H-benzo[d]imidazol-6-yl)-1-((tetrahydro-2H-pyran-4-yl)methyl)-
3,4-dihydropyrazino[2,3-b]pyrazin-2(1H)-one;
7-(2-amino-4-methyl-1 H-benzo[d]imidazol-6-yl)-1-((tetrahydro-2H-pyran-4-yl)methyl)-3,4-
dihydropyrazino[2,3-b]pyrazin-2(1H)-one;
7-(2-methyl-6-(4H-1,2,4-triazol-3-yl)pyridin-3-yl)-1-(2-(tetrahydro-2H-pyran-4-yl)ethyl)-3,4-
dihydropyrazino[2,3-b]pyrazin-2(1H)-one;
(R)-7-(6-(2-hydroxypropan-2-yl)pyridin-3-yl)-3-methyl-1-(2-(tetrahydro-2H-pyran-4-yl)ethyl)-
3,4-dihydropyrazino[2,3-b]pyrazin-2(1H)-one;
(S)-7-(6-(2-hydroxypropan-2-yl)pyridin-3-yl)-3-methyl-1-(2-(tetrahydro-2H-pyran-4-yl)ethyl)-
3,4-dihydropyrazino[2,3-b]pyrazin-2(1H)-one;
7-(6-(2-hydroxypropan-2-yl)pyridin-3-yl)-3,3-dimethyl-1-(2-(tetrahydro-2H-pyran-4-yl)ethyl)-
3,4-dihydropyrazino[2,3-b]pyrazin-2(1H)-one;
7-(2-amino-4-methyl-1H-benzo[d]imidazol-6-yl)-1-(2-(tetrahydro-2H-pyran-4-yl)ethyl)-3,4-
dihydropyrazino[2,3-b]pyrazin-2(1H)-one;
7-(6-(2-hydroxypropan-2-yl)pyridin-3-yl)-1-(2-(tetrahydro-2H-pyran-4-yl)ethyl)-3,4-
dihydropyrazino[2,3-b]pyrazin-2(1H)-one;
7-(2-methyl-4-(1H-1,2,4-triazol-3-yl)phenyl)-1-(2-(tetrahydro-2H-pyran-4-yl)ethyl)-3,4-
dihydropyrazino[2,3-b]pyrazin-2(1H)-one;
7-(4-(1H-1,2,4-triazol-5-yl)phenyl)-1-(2-(tetrahydro-2H-pyran-4-yl)ethyl)-3,4-
dihydropyrazino[2,3-b]pyrazin-2(1H)-one;
1-(1-hydroxypropan-2-yl)-7-(2-methyl-6-(1H-1,2,4-triazol-3-yl)pyridin-3-yl)-3,4-
dihydropyrazino[2,3-b]pyrazin-2(1H)-one; and
1-(2-hydroxyethyl)-7-(2-methyl-6-(1H-1,2,4-triazol-3-yl)pyridin-3-yl)-3,4-dihydropyrazino[2,3-
b]pyrazin-2(1H)-one,
and pharmaceutically acceptable salts, clathrates, solvates, stereoisomers, tautomers, metabolites,
isotopologues and prodrugs thereof.

In one embodiment, the TORK kinase inhibitor is a Compound 1, Compound 2, Compound 3 or Compound 4. In one embodiment, the TOR kinase inhibitor is Compound 1 (a TOR kinase inhibitor set forth herein having molecular formula C₂₁H₂₇N₅O₃). In one embodiment, the TOR kinase inhibitor is Compound 2 (a TOR kinase inhibitor set forth herein having molecular formula C₁₆H₁₆N₈O). In one embodiment, the TOR kinase inhibitor is Compound 3 (a TOR kinase inhibitor set forth herein having molecular formula C₂₁H₂₄N₈O₂). In one embodiment, the TOR kinase inhibitor is Compound 4 (a TOR kinase inhibitor set forth herein having molecular formula C₂₀H₂₅N₅O₃). In one embodiment, Compound 1 is 7-(6-(2-hydroxypropan-2-yl)pyridin-3-yl)-1-((trans)-4-methoxycyclohexyl)-3,4-dihydropyrazino[2,3-b]pyrazin-2(1H)-one, also having the chemical names 7-(6-(2-hydroxypropan-2-yl)pyridin-3-yl)-1-((1r,4r)-4-methoxycyclohexyl)-3,4-dihydropyrazino[2,3-b]pyrazin-2(1H)-one and 7-(6-(2-hydroxypropan-2-yl)pyridin-3-yl)-1-((1R*,4R*)-4-methoxycyclohexyl)-3,4-dihydropyrazino[2,3-b]pyrazin-2(1H)-one, which has the following structure:

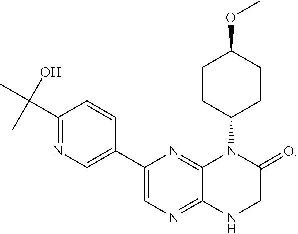 [SMILES: CO[C@H]1CC[C@H](N2C(=O)CNc3ncc(-c4ccc(C(C)(C)O)nc4)nc32)CC1]

In another embodiment, Compound 2 is 1-ethyl-7-(2-methyl-6-(1H-1,2,4-triazol-3-yl)pyridin-3-yl)-3,4-dihydropyrazino[2,3-b]pyrazin-2(1H)-one or a tautomer thereof, for example, 1-ethyl-7-(2-methyl-6-(4H-1,2,4-triazol-3-yl)pyridin-3-yl)-3,4-dihydropyrazino[2,3-b]pyrazin-2(1H)-one, or 1-ethyl-7-(2-methyl-6-(1H-1,2,4-triazol-5-yl)pyridin-3-yl)-3,4-dihydropyrazino[2,3-b]pyrazin-2(1H)-one. In another embodiment, Compound 3 is 7-(2-methyl-6-(4H-1,2,4-triazol-3-yl)pyridin-3-yl)-1-(2-(tetrahydro-2H-pyran-4-yl)ethyl)-3,4-dihydropyrazino[2,3-b]pyrazin-2(1H)-one. In another embodiment, Compound 4 is 1-((trans)-4-hydroxycyclohexyl)-7-(6-(2-hydroxypropan-2-yl)pyridin-3-yl)-3,4-dihydropyrazino[2,3-b]pyrazin-2(1H)-one, alternatively named 1-((1r,4r)-4-hydroxycyclohexyl)-7-(6-(2-hydroxypropan-2-yl)pyridin-3-yl)-3,4-dihydropyrazino[2,3-b]pyrazin-2(1H)-one. In one embodiment, Compound 4 is a metabolite of Compound 1.

### 5.3 Methods for Making TOR Kinase Inhibitors

The TOR kinase inhibitors can be obtained via standard, well-known synthetic methodology, see e.g., March, J. Advanced Organic Chemistry; Reactions Mechanisms, and Structure, 4th ed., 1992. Starting materials useful for preparing compounds of formula (III) and intermediates therefore, are commercially available or can be prepared from commercially available materials using known synthetic methods and reagents.

Particular methods for preparing compounds of formula (I) are disclosed in U.S. Pat. No. 8,110,578, issued Feb. 7, 2012, and U.S. Pat. No. 8,569,494, issued Oct. 29, 2013, incorporated by reference herein in their entirety.

### 5.4 Methods of Use

Provided herein are methods for treating or preventing a cancer characterized by a gene mutation, for example, breast cancer, comprising administering an effective amount of a TOR kinase inhibitor to a patient having a cancer characterized by a particular gene mutation, relative to wild type. Without being limited by theory, it is believed that certain gene mutations correlate with sensitivity to TOR kinase inhibitors, as described herein. In some embodiments described herein, the gene mutation occurs in one or more genes from Table 1, i.e. PIK3CA, RICTOR, TP53, IGF1R or PTEN. In one embodiment, the mutation is a mutation in one or more of RICTOR, TP53 or IGF1R. In some such embodiments, a further mutation is a mutation in PIK3CA. In one embodiment, the mutation is a mutation in the gene sequence of AKT1. In one embodiment, the mutation is a gene amplication mutation in the gene sequence of AKT2. In one embodiment, the mutation is a mutation in RICTOR. In another, the mutation is a mutation in TP53. In yet another, the mutation is a mutation in IGF1R. In some such embodiments, a further mutation results in PTEN loss. In some such embodiments, the breast cancer is ER+. In some such embodiments, the breast cancer is PR+. In other embodiments, the breast cancer is ER+/PR+. Provided herein are also the TOR kinase inhibitors of the present invention for use in methods described herein.

In one embodiment, the gene mutation is a single base change. In another, the gene mutation is a multiple base change. In yet another, the gene mutation is one or more nucleotide insertions. In still another, the gene mutation is one or more nucleotide deletions. In some embodiments, the gene mutation is a copy number change, including loss of one copy or focal or large amplifications of segments of DNA. In yet another embodiment, the gene mutation is a rearrangement of the DNA, wherein the DNA strands break and are rejoined differently from the wild type.

Further provided herein are methods for treating or preventing a cancer characterized by a gene mutation, for example breast cancer, comprising screening a patient's cancer for the presence of a particular gene mutation relative to wild type, and administering an effective amount of a TOR kinase inhibitor to the patient having a cancer characterized by a particular gene mutation.

Further provided herein are methods for predicting response to treatment with a TOR kinase inhibitor in a patient having a cancer characterized by a gene mutation, for example breast cancer, the method comprising: a) obtaining a biological test sample from the patient's cancer; b) obtaining the gene sequence of one or more genes selected from Table 1 in said biological test sample; c) comparing said gene sequence(s) to the gene sequence(s) of a biological wild-type sample; wherein the presence of a mutation indicates an increased likelihood of response to TOR kinase inhibitor treatment of said patient's cancer. In some such embodiments, the method additionally comprises administering an effective amount of a TOR kinase inhibitor, as described herein.

Further provided herein are methods for predicting therapeutic efficacy of TOR kinase inhibitor treatment of a patient having a cancer characterized by a gene mutation, for example breast cancer, with a TOR kinase inhibitor, the method comprising: a) obtaining a biological test sample from the patient's cancer; b) obtaining the gene sequence(s) of one or more genes selected from Table 1 in said biological test sample; c) comparing said gene sequence(s) to the gene sequence(s) of a biological wild-type sample; wherein the presence of a mutation indicates an increased likelihood of therapeutic efficacy of said TOR kinase inhibitor treatment for said patient. In some such embodiments, the method additionally comprises administering an effective amount of a TOR kinase inhibitor, as described herein.

Further provided herein are methods for treating or preventing a breast cancer characterized by a gene mutation, comprising administering an effective amount of a TOR kinase inhibitor to a patient having a breast cancer characterized by a gene mutation, relative to wild type, wherein the gene mutation is a mutation in the gene sequence of AKT1 or a gene amplication mutation in the gene sequence of AKT2.

Further provided herein are methods for for treating or preventing a breast cancer characterized by a gene mutation, comprising screening a patient's breast cancer for the presence of a gene mutation relative to wild type, and administering an effective amount of a TOR kinase inhibitor to the patient having a cancer characterized by a gene mutation, wherein the gene mutation is a mutation in the gene sequence of AKT1 or a gene amplication mutation in the gene sequence of AKT2.

Further provided herein are methods for predicting response to treatment with a TOR kinase inhibitor in a patient having a breast cancer characterized by a gene mutation, the method comprising: a) obtaining a biological test sample from the patient's cancer; b) obtaining the gene sequence of a gene selected from AKT1 and AKT2 in said biological test sample; c) comparing said gene sequence to the gene sequence of a biological wild-type sample; wherein the presence of a mutation in the gene sequence of AKT1 or the presence of a gene amplification mutation in the gene sequence of AKT2 indicates an increased likelihood of response to TOR kinase inhibitor treatment of said patient's cancer.

Further provided herein are methods for predicting therapeutic efficacy of TOR kinase inhibitor treatment of a patient having a breast cancer characterized by a gene mutation, with a TOR kinase inhibitor, the method comprising: a) obtaining a biological test sample from the patient's cancer; b) obtaining the gene sequence of a gene selected from AKT1 and AKT2 in said biological test sample; c) comparing said gene sequence to the gene sequence of a biological wild-type sample; wherein the presence of a mutation in the gene sequence of AKT1 or the presence of a gene amplification mutation in the gene sequence of AKT2 indicates indicates an increased likelihood of therapeutic efficacy of said TOR kinase inhibitor treatment for said patient.

In certain embodiments provided herein, the gene sequence(s) of the biological test sample is obtained using, for example, Sanger sequencing, di-deoxy chain termination sequencing, massively parallel next generation sequencing (NGS), or PCR based methods. In some embodiments, comparison of gene sequences is performed using analytical pipelines that process raw sequence data for tumor samples and reference samples, filter out data artifacts from the sequencing process; filter out known polymorphisms and identify the mutation variants present in the tumor sample.

In one embodiment, the gene mutation or loss results in a decrease in mRNA expression (e.g., relative to wild type). In another embodiment, the gene mutation or loss results in a change in mRNA structure (e.g., relative to wild type). In another embodiment, the gene mutation results in a decrease in protein production (e.g., relative to wild type). In another embodiment, the gene mutation results in a change in protein structure (e.g., relative to wild type). Types of gene mutations contemplated include mutations of the DNA sequence in which the number of bases is altered, categorized as insertion or deletion mutations (including frameshift mutations and full gene deletions), and mutations of the DNA that change one base into another, categorized as missense mutations, which are subdivided into the classes of transitions (one purine to another purine, or one pyrimidine to another pyrimidine) and transversions (a purine to a pyrimidine, or a pyrimidine to a purine) and nonsense mutations, wherein a codon encoding an amino acid is changed to a stop codon, thus resulting in truncated protein. Similarly, mutations comtemplated include copy number alterations wherein one full copy of the gene may be lost (loss of heterozygosity or LOH) or the entire gene may be replicated resulting in an amplified number of gene copies (gene amplification); similarly translocatons where the double strand of DNA is broken and rejoined with a new segment of DNA may result in an altered, truncated or over expressed transcript and protein.

In certain embodiments, the gene mutation(s), for example, in a biological test sample, as referenced herein is present in the sequence(s) of one or more of the genes set forth in Table 1, i.e. in one or more of PIK3CA, RICTOR, TP53, IGF1R and PTEN. In one embodiment, the gene mutation is a mutation in one or more of RICTOR, TP53 or IF1G1. In another embodiment, the gene mutation is a mutation in one or more of RICTOR, TP53 or IGF1R in addition to one or more of the genes set forth in Table 1. In some such embodiments, a further gene mutation is a mutation in PIK3CA. In one embodiment, the mutation is a mutation in the gene sequence of AKT1. In one embodiment, the mutation is a gene amplication mutation in the gene sequence of AKT2.

In one embodiment, the gene mutation is a somatic mutation.

Provided herein are methods for treating or preventing a cancer characterized by one or more gene variants, for example, breast cancer, DLBCL, GBM, HCC, MM, NET, or NSCLC, comprising administering an effective amount of a TOR kinase inhibitor to a patient having a cancer characterized by one or more particular gene variants, relative to wild type. Without being limited by theory, it is believed that certain gene variants correlate with sensitivity to TOR kinase inhibitors, as described herein.

In some embodiments described herein, gene variants occur in one or more genes from FIG. 2. In some embodiments described herein, gene variants occur in one or more genes from Table 2 or Table 3. In some embodiments, the gene variants occur in one or more genes of patients showing a best overall response of Stable Disease (SD), Partial Response (PR) or Non-Progression.

In one embodiment, the variant is one or more known somatic-variants, likely-somatic variants, rearrangements, variants-of-unknown-significance, or copy-number variants, for example, amplifications or deletions, or a combination thereof. In one embodiment, the variant is one or more known somatic variants. In another embodiment, the variant is one or more likely somatic-variants. In one embodiment, the variant is one or more rearrangements. In one embodiment, the variant is one or more variants-of-unknown-significance. In one embodiment, the variant is one or more amplifications. In another embodiment, the variant is one or more deletions.

Further provided herein are methods for treating or preventing a cancer characterized by one or more gene variants, for example breast cancer, DLBCL, GBM, HCC, MM, NET, or NSCLC, comprising screening a patient's cancer for the presence of one or more particular gene variants relative to wild type, for example in one or more genes from FIG. 2, and administering an effective amount of a TOR kinase inhibitor to the patient having a cancer characterized by one or more particular gene variants.

Further provided herein are methods for predicting response to treatment with a TOR kinase inhibitor in a patient having a cancer characterized by one or more gene variants, for example breast cancer, DLBCL, GBM, HCC, MM, NET, or NSCLC, the method comprising: a) obtaining a biological test sample from the patient's cancer; b) obtaining the gene sequence of the genes listed in FIG. 2 in said biological test sample; c) comparing said gene sequence(s) to the gene sequence(s) of a biological wild-type sample; wherein the presence of one or more variants in one or more genes selected from FIG. 2, Table 2 or Table 3 indicates an increased likelihood of response to TOR kinase inhibitor treatment of said patient's cancer. In some such embodiments, the method additionally comprises administering an effective amount of a TOR kinase inhibitor, as described herein.

Further provided herein are methods for predicting response to treatment with a TOR kinase inhibitor in a patient having a cancer characterized by one or more gene variants, for example breast cancer, DLBCL, GBM, HCC, MM, NET, or NSCLC, the method comprising: a) obtaining a biological test sample from the patient's cancer; b) obtaining the gene sequence of one or more genes selected from Table 2 or Table 3 in said biological test sample; c) comparing said gene sequence(s) to the gene sequence(s) of a biological wild-type sample; wherein the presence of one or more variants indicates an increased likelihood of response to TOR kinase inhibitor treatment of said patient's cancer. In some such embodiments, the method additionally comprises administering an effective amount of a TOR kinase inhibitor, as described herein.

Further provided herein are methods for predicting therapeutic efficacy of TOR kinase inhibitor treatment of a patient having a cancer characterized by one ore more gene variants, for example breast cancer, DLBCL, GBM, HCC, MM, NET, or NSCLC, with a TOR kinase inhibitor, the method comprising: a) obtaining a biological test sample from the patient's cancer; b) obtaining the gene sequence(s) of the genes listed in FIG. 2 in said biological test sample; c) comparing said gene sequence(s) to the gene sequence(s) of a biological wild-type sample; wherein the presence of one or more variants of one or more genes selected from FIG. 2, Table 2 or Table 3 indicates an increased likelihood of therapeutic efficacy of said TOR kinase inhibitor treatment for said patient. In some such embodiments, the method additionally comprises administering an effective amount of a TOR kinase inhibitor, as described herein.

Further provided herein are methods for predicting therapeutic efficacy of TOR kinase inhibitor treatment of a patient having a cancer characterized by one ore more gene variants, for example breast cancer, DLBCL, GBM, HCC, MM, NET, or NSCLC, with a TOR kinase inhibitor, the method comprising: a) obtaining a biological test sample from the patient's cancer; b) obtaining the gene sequence(s) of one or more genes selected from Table 2 or Table 3 in said biological test sample; c) comparing said gene sequence(s) to the gene sequence(s) of a biological wild-type sample; wherein the presence of one or more variants indicates an increased likelihood of therapeutic efficacy of said TOR kinase inhibitor treatment for said patient. In some such embodiments, the method additionally comprises administering an effective amount of a TOR kinase inhibitor, as described herein.

In certain embodiments provided herein, the gene sequence(s) of the biological test sample is obtained using, for example, Sanger sequencing, di-deoxy chain termination sequencing, massively parallel next generation sequencing (NGS), or PCR based methods. In some embodiments, comparison of gene sequences is performed using analytical pipelines that process raw sequence data for tumor samples and reference samples, filter out data artifacts from the sequencing process; filter out known polymorphisms and identify the variants present in the tumor sample.

In one embodiment, the gene variant results in a decrease in mRNA expression (e.g., relative to wild type). In another embodiment, the gene variant results in a change in mRNA structure (e.g., relative to wild type). In another embodiment, the gene variant results in a decrease in protein production (e.g., relative to wild type). In another embodiment, the gene variant results in a change in protein structure (e.g., relative to wild type). Types of gene variants contemplated include mutations of the DNA sequence in which the number of bases is altered, categorized as insertion or deletion mutations (including frameshift mutations and full gene deletions), and mutations of the DNA that change one base into another, categorized as missense mutations, which are subdivided into the classes of transitions (one purine to another purine, or one pyrimidine to another pyrimidine) and transversions (a purine to a pyrimidine, or a pyrimidine to a purine) and nonsense mutations, wherein a codon encoding an amino acid is changed to a stop codon, thus resulting in truncated protein. Similarly, variants comtemplated include copy number alterations wherein one full copy of the gene may be lost (loss of heterozygosity or LOH) or the entire gene may be replicated resulting in an amplified number of gene copies (gene amplification); similarly translocatons where the double strand of DNA is broken and rejoined with a new segment of DNA may result in an altered, truncated or over expressed transcript and protein.

In certain embodiments, the gene variant(s), for example, in a biological test sample, as referenced herein, is present in the sequence(s) of one or more of the genes set forth in FIG. 2. In certain embodiments, the gene variant(s), for example, in a biological test sample, as referenced herein, is present in the sequence(s) of one or more of the genes set forth in Table 2 or Table 3.

In some embodiments, the gene variant(s), for example in a biological test sample, as referenced herein, is present in one or more of AKT1, AKT2, AKT3, ARID1A, NF1, PHLPP2, PIK3CA, PIK3R1, PTEN, RICTOR, RPTOR, STK11 (LKB1), TSC1, TSC2, PDK1, PRAS40, PRKDC, EIF4E, and EIF4EBP1.

In some embodiments, the gene variant(s), for example in a biological test sample, as referenced herein, is not present in one or more of EGFR, IGF1R, IGF2R, KRAS, MYC, ERBB3, MET, PDGFRB, NOTCH1, MEK, BRAF, N-RAS, MAP3K8, BCL2, BCL2L11, BAD, MCL1, BIRC5, CCND1, ARAF, RAF1, CDC25A, MDM2, FOXO3, GSK3B, and XIAP. In some such embodiments, the patient has a variant in one or more genes from Table 2 or Table 3.

In some embodiments, the gene variant(s), for example in a biological test sample, as referenced herein, is present in one or more of EGFR, ERBB2 (HER2), KIT, PDGFRA, PIK3CA, PTEN, DAXX, ATRX, MEN1, FGFR4, ARID1A, KDMA6A, TP53, FGFR3, NF2, TSC1, CDKN2A, or MCL1. In some embodiments, the gene variant(s), for example in a biological test sample from a NET patient, is present in one or more of EGFR, ERBB2 (HER2), KIT, PDGFRA, PIK3CA and PTEN. In some embodiments, the gene variant(s), for example in a biological test sample from a NET patient, is present in one or more of DAXX, ATRX, MEN1, PIK3CA, PTEN, TP53, TSC2, and FGFR4. In some embodiments, the gene variant(s), for example in a biological test sample from a breast cancer patient, is present in one or more of PIK3CA, PTEN, ARID1A, and MCL1. In some embodiments, the gene variant(s), for example in a biological test sample from a metastatic bladder cancer patient, is present in one or more of ARID1A, KDMA6A, TP53, FGFR3, NF2, and TSC1. In some embodiments, the gene variant(s), for example in a biological test sample from a glioblastoma patient, is present in one or more of PDGFRA, and CDKN2A.

In some embodiments, the gene variant(s), for example in a biological test sample, as referenced herein, is present in one or more of ARID1A, CEBPA, FGFR2, IGF1R, RICTOR, STK11, GPR124, TNFAIP3, CARD11, FANCA, KIT, JAK2 and BRAF. In some embodiments, the gene variant(s), for example in a biological test sample from a HCC patient, is present in one or more of ARID1A and CEBPA. In some embodiments, the gene variant(s), for example in a biological test sample from a solid tumor patient, is present in one or more of ARID1A, FGFR2, IGF1R, RICTOR, and STK11. In some embodiments, the gene variant(s), for example in a biological test sample from a HCC patient, is present in GPR124. In some embodiments, the gene variant(s), for example in a biological test sample from a solid tumor patient, is present in GPR124. In some embodiments, the gene variant(s), for example in a biological test sample from a NSCLC patient, is present in one or more of TNFAIP3, APC, ARID1A, CARD11, FANCA, and KIT. In some embodiments, the gene variant(s), for example in a biological test sample from a DLBCL patient, is present in JAK2.

In one embodiment, a patient or a patient's cancer is screened for gene mutation or variant(s) by obtaining a biological sample from said patient or said patient's cancer, and determining the gene sequence(s) of said sample ex vivo. In certain embodiments, the ex vivo analysis is performed using microarray analysis or sequence based techniques, for example, Sanger sequencing, di-deoxy chain termination sequencing, massively parallel next generation sequencing (NGS), or PCR based methods. Examples of traditional DNA sequencing methods include Sanger sequencing (chain termination); pyrosequencing (sequencing by synthesis method); mass spectroscopy-based mutation analysis (MALDI-TOF); allele-specific RT PCR; and RT-PCR melting curve analysis. NGS methods include flow-based, reversible dye termination and 4-color optical imaging; emulsion PCR with bead-based pyrosequencing and charge-coupled device (C≡CD) light imaging; oligo-dT captured PolyA-tailed DNA fragments, flow cell 4-color deoxynucleotide phosphate (dNTP) optical imaging; sequential dinucleotide ligation, flow cell-based 4-color optical imaging; and semiconductor-based nonoptical detection, standard dNTP sequencing chemistry (see J. Ross and M. Cronin, Am. J. Clin. Pathol, 136; 527-539 (2011)).

In further embodiments, the cancer characterized by a gene mutation or variant(s), for example, breast cancer, DLBCL, GBM, HCC, MM, NET, or NSCLC, is that in which the PI3K/mTOR pathway is activated. In certain embodiments, the cancer characterized by a gene mutation or variant(s), for example breast cancer, DLBCL, GBM, HCC, MM, NET, or NSCLC, is that in which the PI3K/mTOR pathway is activated due to PTEN loss, a PIK3CA mutation or EGFR overexpression, or a combination thereof.

In other embodiments, the cancer characterized by a gene mutation or variant(s), for example breast cancer, DLBCL, GBM, HCC, MM, NET, or NSCLC, is a cancer associated with the pathways involving mTOR, PI3K, or Akt kinases and mutants or isoforms thereof. Other cancers within the scope of the methods provided herein include those associated with the pathways of the following kinases: PI3Kα, PI3Kβ, PI3Kδ, KDR, GSK3α, GSK3β, ATM, ATX, ATR, cFMS, and/or DNA-PK kinases and mutants or isoforms thereof.

In one embodiment, provided herein are methods for achieving a Response Evaluation Criteria in Solid Tumors (for example, RECIST 1.1) of complete response, partial response or stable disease in a patient comprising administering an effective amount of a TOR kinase inhibitor to a patient having a cancer characterized by a gene mutation or variant(s), for example breast cancer, DLBCL, GBM, HCC, MM, NET, or NSCLC. In one such embodiment, the variant is in one or more of ARID1A, CEBPA, FGFR2, IGF1R, RICTOR or STK11. In some such embodiments, the patient is a HCC patient, and the variant is in ARID1A, CEBPA or both. In some such embodiments, the patient is a solid tumor patient, and the variant is in one or more of ARID1A, FGFR2, IGF1R, RICTOR, and STK11. In another such embodiment, the variant is in GPR124. In some such embodiments, the patient is a solid tumor patient, for example, an HCC patient. In another embodiment, provided herein are methods to increase Progression Free Survival rates, as determined by Kaplan-Meier estimates. In some such embodiments, the variant is in one or more of APC, ARID1A, CARD11, FANCA, KIT, and JAK2. In some such embodiments, the patient is an NSCLC patient and the variant is in one or more of APC, ARID1A, CARD11, FANCA, and KIT. In another such embodiment, the patient is a DLBCL patient and the variant is in JAK2.

Further provided herein are methods for treating or preventing a hematological cancer, for example DLBCL (diffuse large B-cell lymphoma), ML (mantle cell lymphoma), FL (follicular lymphoma), and AML (acute myeloid leukemia), characterized by decreased IRF4 gene and/or protein expression, comprising screening a patient's cancer for the presence of decreased IRF4 gene and/or protein expression relative to wild type, and administering an effective amount of a TOR kinase inhibitor to the patient having a cancer characterized by low IRF4 gene and/or protein expression.

Further provided herein are methods for predicting response to treatment with a TOR kinase inhibitor in a patient having a hematological cancer, for example DLBCL (diffuse large B-cell lymphoma), ML (mantle cell lymphoma), FL (follicular lymphoma), and AML (acute myeloid leukemia), characterized by decreased IRF4 gene and/or protein expression, the method comprising: a) obtaining a biological test sample from the patient's cancer; b) obtaining the IRF-4 gene and/or protein expression levels in said biological test sample; c) comparing said IRF4 gene and/or protein expression levels to the IRF4 gene and/or protein expression levels of a biological wild-type sample; wherein a decreased IRF4 gene and/or protein expression level indicates an increased likelihood of response to TOR kinase inhibitor treatment of said patient's cancer. In some such embodiments, the method additionally comprises administering an effective amount of a TOR kinase inhibitor, as described herein.

Further provided herein are methods for predicting therapeutic efficacy of TOR kinase inhibitor treatment of a patient having a hematological cancer, for example DLBCL (diffuse large B-cell lymphoma), ML (mantle cell lymphoma), FL (follicular lymphoma), and AML (acute myeloid leukemia), characterized by decreased IRF4 gene and/or protein expression, the method comprising: a) obtaining the IRF-4 gene and/or protein expression levels in said biological test sample; c) comparing said IRF-4 gene and/or protein expression levels to the IRF4 gene and/or protein expression levels of a biological wild-type sample; wherein a decreased IRF4 gene and/or protein level indicates an increased likelihood of therapeutic efficacy of said TOR kinase inhibitor treatment for said patient. In some such embodiments, the method additionally comprises administering an effective amount of a TOR kinase inhibitor, as described herein.

Further provided herein are methods for treating or preventing a hematological cancer, for example DLBCL, characterized by increased levels of TOR pathway activation, for example, increased levels of one or more of p-mTOR S2448, p-p70S6K T389, pGSK3b S9 and S21, pAKT 5473 and T308, pTSC2 T1462, and pS6 S240/S244 and S235/S236, comprising screening a patient's cancer for the presence of increased levels of TOR pathway activation relative to wild type, for example, increased levels of one or more of p-mTOR S2448, p-p70S6K T389, pGSK3b S9 and S21, pAKT S473 and T308, pTSC2 T1462, and pS6 S240/S244 and S235/S236 relative to wild type, and administering an effective amount of a TOR kinase inhibitor to the patient having a cancer characterized by increased levels of TOR pathway activation, for example, increased levels of one or more of p-mTOR S2448, p-p70S6K T389, pGSK3b S9 and S21, pAKT S473 and T308, pTSC2 T1462, and pS6 S240/S244 and S235/S236.

Further provided herein are methods for predicting response to treatment with a TOR kinase inhibitor in a patient having a hematological cancer, for example DLBCL, characterized by increased levels of TOR pathway activation, for example, increased levels of one or more of p-mTOR S2448, p-p70S6K T389, pGSK3b S9 and S21, pAKT S473 and T308, pTSC2 T1462, and pS6 S240/S244 and S235/S236, the method comprising: a) obtaining a biological test sample from the patient's cancer; b) obtaining the TOR pathway activation levels, for example, the levels of one or more of p-mTOR S2448, p-p70S6K T389, pGSK3b S9 and S21, pAKT 5473 and T308, pTSC2 T1462, and pS6 S240/S244 and S235/S236, in said biological test sample; c) comparing said TOR pathway activation levels, for example, the levels of one or more of p-mTOR S2448, p-p70S6K T389, pGSK3b S9 and S21, pAKT S473 and T308, pTSC2 T1462, and pS6 S240/S244 and S235/S236, to the TOR pathway activation levels, for example, the levels of one or more of p-mTOR S2448, p-p70S6K T389, pGSK3b S9 and S21, pAKT S473 and T308, pTSC2 T1462, and pS6 S240/S244 and S235/S236, of a biological wild-type sample; wherein an increased TOR pathway activation level, for example, increased levels of one or more of p-mTOR S2448, p-p70S6K T389, pGSK3b S9 and S21, pAKT S473 and T308, pTSC2 T1462, and pS6 S240/S244 and S235/S236, indicates an increased likelihood of response to TOR kinase inhibitor treatment of said patient's cancer. In some such embodiments, the method additionally comprises administering an effective amount of a TOR kinase inhibitor, as described herein.

Further provided herein are methods for predicting therapeutic efficacy of TOR kinase inhibitor treatment of a patient having a cancer characterized by increased levels of TOR pathway activation, for example, increased levels of one or more of p-mTOR S2448, p-p70S6K T389, pGSK3b S9 and S21, pAKT S473 and T308, pTSC2 T1462, and pS6 S240/S244 and S235/S236, the method comprising: a) obtaining the TOR pathway activation levels, for example, the levels of one or more of p-mTOR S2448, p-p70S6K T389, pGSK3b S9 and S21, pAKT S473 and T308, pTSC2 T1462, and pS6 S240/S244 and S235/S236, in said biological test sample; c) comparing said TOR pathway activation levels, for example, the levels of one or more of p-mTOR S2448, p-p70S6K T389, pGSK3b S9 and S21, pAKT S473 and T308, pTSC2 T1462, and pS6 S240/S244 and S235/S236, to the TOR pathway activation levels, for example, the levels of one or more of p-mTOR S2448, p-p70S6K T389, pGSK3b S9 and S21, pAKT S473 and T308, pTSC2 T1462, and pS6 S240/S244 and S235/S236, of a biological wild-type sample; wherein an increased TOR pathway activation level, for example, increased levels of one or more of p-mTOR S2448, p-p70S6K T389, pGSK3b S9 and S21, pAKT S473 and T308, pTSC2 T1462, and pS6 S240/S244 and S235/S236 indicates an increased likelihood of therapeutic efficacy of said TOR kinase inhibitor treatment for said patient. In some such embodiments, the method additionally comprises administering an effective amount of a TOR kinase inhibitor, as described herein.

In one embodiment, provided herein are methods for preventing or delaying a Response Evaluation Criteria in Solid Tumors (for example, RECIST 1.1) of progressive disease in a patient, comprising administering an effective amount of a TOR kinase inhibitor to a patient having a cancer characterized by a gene mutation or variant(s), for example breast cancer, DLBCL, GBM, HCC, MM, NET, or NSCLC. In one embodiment the prevention or delaying of progressive disease is characterized or achieved by a change in overall size of the target lesions, of for example, between −30% and +20% compared to pre-treatment. In another embodiment, the change in size of the target lesions is a reduction in overall size of more than 30%, for example, more than 50% reduction in target lesion size compared to pre-treatment. In some such embodiments, the patient is a NSCLC patient and the variant is in TNFAIP3. In another, the prevention is characterized or achieved by a reduction in size or a delay in progression of non-target lesions compared to pre-treatment. In one embodiment, the prevention is achieved or characterized by a reduction in the number of target lesions compared to pre-treatment. In another, the prevention is achieved or characterized by a reduction in the number or quality of non-target lesions compared to pre-treatment. In one embodiment, the prevention is achieved or characterized by the absence or the disappearance of target lesions compared to pre-treatment. In another, the prevention is achieved or characterized by the absence or the disappearance of non-target lesions compared to pre-treatment. In another embodiment, the prevention is achieved or characterized by the prevention of new lesions compared to pre-treatment. In yet another embodiment, the prevention is achieved or characterized by the prevention of clinical signs or symptoms of disease progression compared to pre-treatment, such as cancer-related cachexia or increased pain.

In certain embodiments, provided herein are methods for decreasing the size of target lesions in a patient compared to pre-treatment, comprising administering an effective amount of a TOR kinase inhibitor to a patient having a cancer characterized by a gene mutation or variant(s), for example breast cancer, DLBCL, GBM, HCC, MM, NET, or NSCLC.

In certain embodiments, provided herein are methods for decreasing the size of a non-target lesion in a patient compared to pre-treatment, comprising administering an effective amount of a TOR kinase inhibitor to a patient having a cancer characterized by a gene mutation or variant(s), for example breast cancer, DLBCL, GBM, HCC, MM, NET, or NSCLC.

In certain embodiments, provided herein are methods for achieving a reduction in the number of target lesions in a patient compared to pre-treatment, comprising administering an effective amount of a TOR kinase inhibitor to a patient having a cancer characterized by a gene mutation or variant(s), for example breast cancer, DLBCL, GBM, HCC, MM, NET, or NSCLC.

In certain embodiments, provided herein are methods for achieving a reduction in the number of non-target lesions in a patient compared to pre-treatment, comprising administering an effective amount of a TOR kinase inhibitor to a patient having a cancer characterized by a gene mutation or variant(s), for example breast cancer, DLBCL, GBM, HCC, MM, NET, or NSCLC.

In certain embodiments, provided herein are methods for achieving an absence of all target lesions in a patient, comprising administering an effective amount of a TOR kinase inhibitor to a patient having a cancer characterized by a gene mutation or variant(s), for example breast cancer, DLBCL, GBM, HCC, MM, NET, or NSCLC.

In certain embodiments, provided herein are methods for achieving an absence of all non-target lesions in a patient, comprising administering an effective amount of a TOR kinase inhibitor to a patient having a cancer characterized by a gene mutation or variant(s), for example breast cancer, DLBCL, GBM, HCC, MM, NET, or NSCLC.

In certain embodiments, provided herein are methods for treating a cancer characterized by a gene mutation or variant(s), for example breast cancer, DLBCL, GBM, HCC, MM, NET, or NSCLC, the methods comprising administering an effective amount of a TOR kinase inhibitor to a patient having a cancer characterized by a gene mutation or variant(s), for example breast cancer, DLBCL, GBM, HCC, MM, NET, or NSCLC, wherein the treatment results in a complete response, partial response or stable disease, as determined by Response Evaluation Criteria in Solid Tumors (for example, RECIST 1.1).

In certain embodiments, provided herein are methods for treating a cancer characterized by a gene mutation or variant(s), for example breast cancer, DLBCL, GBM, HCC, MM, NET, or NSCLC, the methods comprising administering an effective amount of a TOR kinase inhibitor to a patient a cancer characterized by a gene mutation or variant(s), for example breast cancer, DLBCL, GBM, HCC, MM, NET, or NSCLC, wherein the treatment results in a reduction in target lesion size, a reduction in non-target lesion size and/or the absence of new target and/or non-target lesions, compared to pre-treatment.

In certain embodiments, provided herein are methods for treating a cancer characterized by a gene mutation or variant(s), for example breast cancer, DLBCL, GBM, HCC, MM, NET, or NSCLC, the methods comprising administering an effective amount of a TOR kinase inhibitor to a patient having a cancer characterized by a gene mutation or variant(s), for example breast cancer, DLBCL, GBM, HCC, MM, NET, or NSCLC, wherein the treatment results in prevention or retarding of clinical progression, such as cancer-related cachexia or increased pain.

In another embodiment, provided herein are methods for improving the Eastern Cooperative Oncology Group Performance Status (ECOG) of a patient, comprising administering an effective amount of a TOR kinase inhibitor to a patient having a cancer characterized by a gene mutation or variant(s), for example breast cancer, DLBCL, GBM, HCC, MM, NET, or NSCLC.

In another embodiment, provided herein are methods for inducing a therapeutic response assessed by Positron Emission Tomography (PET) outcome of a patient, comprising administering an effective amount of a TOR kinase inhibitor to a patient having a cancer characterized by a gene mutation or variant(s), for example breast cancer, DLBCL, GBM, HCC, MM, NET, or NSCLC. In certain embodiments, provided herein are methods for treating a cancer characterized by a gene mutation or variant(s), for example breast cancer, DLBCL, GBM, HCC, MM, NET, or NSCLC, the methods comprising administering an effective amount of a TOR kinase inhibitor to a patient having a cancer characterized by a gene mutation or variant(s), for example breast cancer, DLBCL, GBM, HCC, MM, NET, or NSCLC, wherein the treatment results in a reduction in tumor metabolic activity, for example, as measured by FDG-PET imaging.

In one embodiment, provided herein are methods for inhibiting phosphorylation of S6RP, 4E-BP1 and/or AKT in a patient having a cancer characterized by a gene mutation or variant(s), for example breast cancer, DLBCL, GBM, HCC, MM, NET, or NSCLC, comprising administering an effective amount of a TOR kinase inhibitor to said patient. In some such embodiments, the inhibition of phosphorylation is assessed in a biological sample of the patient, such as in circulating blood and/or tumor cells, skin biopsies and/or tumor biopsies or aspirate. In such embodiments, the amount of inhibition of phosphorylation is assessed by comparison of the amount of phospho-S6RP, 4E-BP1 and/or AKT before and after administration of the TOR kinase inhibitor. In certain embodiments, provided herein are methods for measuring inhibition of phosphorylation of S6RP, 4E-BP1 or AKT in a patient a cancer characterized by a gene mutation or variant(s), for example breast cancer, DLBCL, GBM, HCC, MM, NET, or NSCLC, comprising administering an effective amount of a TOR kinase inhibitor to said patient, measuring the amount of phosphorylated S6RP, 4E-BP1 and/or AKT in said patient, and comparing said amount of phosphorylated S6RP, 4E-BP1 and/or AKT to that of said patient prior to administration of an effective amount of a TOR kinase inhibitor. In some embodiments, the inhibition of phosphorylation of S6RP, 4E-BP1 and/or AKT is assessed in B-cells, T-cells and/or monocytes.

In certain embodiments, provided herein are methods for inhibiting phosphorylation of S6RP, 4E-BP1 and/or AKT in a biological sample of a patient having a cancer characterized by a gene mutation or variant(s), for example breast cancer, DLBCL, GBM, HCC, MM, NET, or NSCLC, comprising administering an effective amount of a TOR kinase inhibitor to said patient and comparing the amount of phosphorylated S6RP, 4E-BP1 and/or AKT in a biological sample of a patient obtained prior to and after administration of said TOR kinase inhibitor, wherein less phosphorylated S6RP, 4E-BP1 and/or AKT in said biological sample obtained after administration of said TOR kinase inhibitor relative to the amount of phosphorylated S6RP, 4E-BP1 and/or AKT in said biological sample obtained prior to administration of said TOR kinase inhibitor indicates inhibition. In some embodiments, the inhibition of phosphorylation of S6RP, 4E-BP1 and/or AKT is assessed in B-cells, T-cells and/or monocytes. Inhibition of phosphorylation of S6RP (Ser235/236 and/or Ser240/244), 4E-BP1 (Thr37/46), and/or AKT (Ser473) can be measured by various methodology including flow cytometry, ELISA, immunohistochemistry (IHC), immunofluorescence (IF) using phosphorylation-specific antibodies.

In one embodiment, provided herein are methods for inhibiting DNA-dependent protein kinase (DNA-PK) activity in a patient having a cancer characterized by a gene mutation or variant(s), for example breast cancer, DLBCL, GBM, HCC, MM, NET, or NSCLC, comprising administering an effective amount of a TOR kinase inhibitor to said patient. In some embodiments, DNA-PK inhibition is assessed in the skin of the patient having a cancer characterized by a gene mutation or variant(s), for example breast cancer, DLBCL, GBM, HCC, MM, NET, or NSCLC, in one example in a UV light-irradiated skin sample of said patient. In another embodiment, DNA-PK inhibition is assessed in a tumor biopsy or aspirate of a patient a cancer characterized by a gene mutation or variant(s), for example breast cancer, DLBCL, GBM, HCC, MM, NET, or NSCLC. In one embodiment, inhibition is assessed by measuring the amount of phosphorylated DNA-PK S2056 (also known as pDNA-PK S2056) before and after administration of the TOR kinase inhibitor. In certain embodiments, provided herein are methods for measuring inhibition of phosphorylation of DNA-PK S2056 in a skin sample of a patient a cancer characterized by a gene mutation or variant(s), for example breast cancer, DLBCL, GBM, HCC, MM, NET, or NSCLC, comprising administering an effective amount of a TOR kinase inhibitor to said patient, measuring the amount of phosphorylated DNA-PK S2056 present in the skin sample and comparing said amount of phosphorylated DNA-PK S2056 to that in a skin sample from said patient prior to administration of an effective amount of a TOR kinase inhibitor. In one embodiment, the skin sample is irradiated with UV light.

In certain embodiments, provided herein are methods for inhibiting DNA-dependent protein kinase (DNA-PK) activity in a skin sample of a patient having a cancer characterized by a gene mutation or variant(s), for example breast cancer, DLBCL, GBM, HCC, MM, NET, or NSCLC, comprising administering an effective amount of a TOR kinase inhibitor to said patient and comparing the amount of phosphorylated DNA-PK in a biological sample of a patient obtained prior to and after administration of said TOR kinase inhibitor, wherein less phosphorylated DNA-PK in said biological sample obtained after administration of said TOR kinase inhibitor relative to the amount of phosphorylated DNA-PK in said biological sample obtained prior to administration of said TOR kinase inhibitor indicates inhibition. Inhibition of DNA-PK activity can be measured by monitoring phosphorylation of substrates of DNA-PK, such as DNA-PK itself and XRCC4. Inhibition of DNA-PK activity can also be measured by monitoring accumulation of double strand DNA damage in tissues and/or cells such as those mentioned above.

In some embodiments, provided herein are methods for treating a cancer characterized by a gene mutation or variant(s), for example breast cancer, DLBCL, GBM, HCC, MM, NET, or NSCLC, the methods comprising administering an effective amount of a TOR kinase to a patient having said cancer, wherein the treatment results in one or more of inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), and/or increased Overall Survival (OS), among others.

In some embodiments, the TOR kinase inhibitor is a compound as described herein. In one embodiment, the TOR kinase inhibitor is a compound of formula (I). In one embodiment, the TOR kinase inhibitor is a compound from Table A. In one embodiment, the TOR kinase inhibitor is Compound 1 (a TOR kinase inhibitor set forth herein having molecular formula C₂₁H₂₇N₅O₃). In one embodiment, the TOR kinase inhibitor is Compound 2 (a TOR kinase inhibitor set forth herein having molecular formula C₁₆H₁₆N₈O). In one embodiment, the TOR kinase inhibitor is Compound 3 (a TOR kinase inhibitor set forth herein having molecular formula C₂₁H₂₄N₈O₂). In one embodiment, the TOR kinase inhibitor is Compound 4 (a TOR kinase inhibitor set forth herein having molecular formula C₂₀H₂₅N₅O₃).

In one embodiment, Compound 1 is 7-(6-(2-hydroxypropan-2-yl)pyridin-3-yl)-1-((trans)-4-methoxycyclohexyl)-3,4-dihydropyrazino[2,3-b]pyrazin-2(1H)-one, also having the chemical names 7-(6-(2-hydroxypropan-2-yl)pyridin-3-yl)-1-((1r,4r)-4-methoxycyclohexyl)-3,4-dihydropyrazino[2,3-b]pyrazin-2(1H)-one and 7-(6-(2-hydroxypropan-2-yl)pyridin-3-yl)-1-((1R*,4R*)-4-methoxycyclohexyl)-3,4-dihydropyrazino[2,3-b]pyrazin-2(1H)-one, which has the following structure:

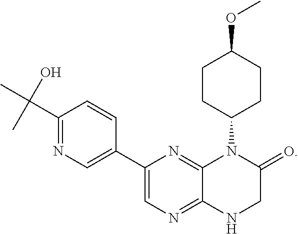 [SMILES: CO[C@H]1CC[C@H](N2C(=O)CNc3ncc(-c4ccc(C(C)(C)O)nc4)nc32)CC1]

In another embodiment, Compound 2 is 1-ethyl-7-(2-methyl-6-(1H-1,2,4-triazol-3-yl)pyridin-3-yl)-3,4-dihydropyrazino[2,3-b]pyrazin-2(1H)-one or a tautomer thereof, for example, 1-ethyl-7-(2-methyl-6-(4H-1,2,4-triazol-3-yl)pyridin-3-yl)-3,4-dihydropyrazino[2,3-b]pyrazin-2(1H)-one, or 1-ethyl-7-(2-methyl-6-(1H-1,2,4-triazol-5-yl)pyridin-3-yl)-3,4-dihydropyrazino[2,3-b]pyrazin-2(1H)-one. In another embodiment, Compound 3 is 7-(2-methyl-6-(4H-1,2,4-triazol-3-yl)pyridin-3-yl)-1-(2-(tetrahydro-2H-pyran-4-yl)ethyl)-3,4-dihydropyrazino[2,3-b]pyrazin-2(1H)-one. In another embodiment, Compound 4 is 1-((trans)-4-hydroxycyclohexyl)-7-(6-(2-hydroxypropan-2-yl)pyridin-3-yl)-3,4-dihydropyrazino[2,3-b]pyrazin-2(1H)-one, alternatively named 1-((1r,4r)-4-hydroxycyclohexyl)-7-(6-(2-hydroxypropan-2-yl)pyridin-3-yl)-3,4-dihydropyrazino[2,3-b]pyrazin-2(1H)-one. In one embodiment, Compound 4 is a metabolite of Compound 1.

Further provided herein are methods for treating patients who have been previously treated for a cancer, as well as those who have not previously been treated. Further provided herein are methods for treating patients who have undergone surgery in an attempt to treat a cancer, as well as those who have not. Because patients with a cancer have heterogenous clinical manifestations and varying clinical outcomes, the treatment given to a patient may vary, depending on his/her prognosis. The skilled clinician will be able to readily determine without undue experimentation specific secondary agents (see for example U.S. Provisional Application Nos. 61/980,124 and 61/980,125, each incorporated herein in their entirety), types of surgery, and types of non-drug based standard therapy that can be effectively used to treat an individual patient with a cancer. In some embodiments, a TOR kinase inhibitor is administered to a patient in combination with 5-azacitidine or erlotinib. In some such embodiments, the patient is an NSCLC patient, wherein the NSCLC is characterized by known somatic variants in one or more of ARID2, CDKN2A/B, FAM123B, KDM5C, KEAP1, KRAS, LRP1B, ROS1, SMARCD1, STK11, or TP53. In some embodiments, the patient is an NSCLC patient, wherein the NSCLC is characterized by amplification variants in one or more of CDK6, EGFR, MCL1 or RICTOR. In some embodiments, the patient is an NSCLC patient, wherein the NSCLC is characterized by one or more variants of unknown significance in one or more of ALOX12B, ATR, BCL6, BRAF, CDH1, CDK6, EPHA5, ERBB4, FANCM, FAT3, FGF4, FGF6, FGFR1, FGFR2, FGFR3, FLT1, FLT4, GATA2, GPR124, GSK3B, IK3R2, IL7R, IRF4, IRS2, JAK1, KDR, KEAP1, LRP1B, MLL, MLL2, MYCN, NOTCH4, NSD1, NTRK1, NUP93, PDGFRA, PIK3CG, RAD51C, RARA, RET, SOCS1, TBX3, TET2, TIPARP, TRRAP, or TSC1.

In some such embodiments, the TOR kinase inhibitor is administered to a patient in combination with 5-azacitidine, wherein the patient is an NSCLC patient, wherein the NSCLC is characterized by known somatic variants in one or more of KEAP1, KRAS, ROS1, or STK11. In some embodiments, the patient is an NSCLC patient, wherein the NSCLC is characterized by one or more variants of unknown significance in one or more of ALOX12B, CDH1, ERBB4, FAT3, FGF4, FGF6, IL7R, IRF4, JAK1, LRP1B, MLL, MLL2, NSD1, NTRK1, PDGFRA, SOCS1, TBX3, TET2, or TSC1.

In some such embodiments, the TOR kinase inhibitor is administered to a patient in combination with erlotinib, wherein the patient is an NSCLC patient, wherein the NSCLC is characterized by known somatic variants in one or more of ARID2, CDKN2A/B, FAM123B, KDM5C, LRP1B, SMARCD1, STK11, or TP53. In some such embodiments, the patient is an NSCLC patient, wherein the NSCLC is characterized by amplification variants in one or more of CDK6, EGFR, MCL1 or RICTOR. In some other such embodiments, the patient is an NSCLC patient, wherein the NSCLC is characterized by one or more variants of unknown significance in one or more of ATR, BCL6, BRAF, CDK6, EPHA5, ERBB4, FANCM, FAT3, FGFR1, FGFR2, FGFR3, FLT1, FLT4, GATA2, GPR124, GSK3B, IK3R2, IRS2, KDR, KEAP1, LRP1B, MLL2, MYCN, NOTCH4, NUP93, PIK3CG, RAD51C, RARA, RET, TIPARP, or TRRAP. In some such embodiments, the patient is an NSCLC patient, wherein the NSCLC is characterized by an EGFR mutation.

In other embodiments, a TOR kinase inhibitor is administered to a patient in combination with an IMiD® immunomodulatory compound. IMiD® immunomodulatory drugs include, but are not limited to, lenalidomide (REVLIMID®) pomalidomide (Actimid™; POMALYST®), (S)-3-(4-(4-(morpholinomethyl)benzyloxy)-1-oxoisoindolin-2-yl)piperidine-2,6-dione, N-[2-(2,6-dioxo-piperidin-3-yl)-1-oxo2,3-dihydro-1H-isoindol-4-ylmethyl]-2-phenyl-acetamide, 2-(2,6-dioxopiperidin-3-yl)-4-phenylaminoisoindole-1,3-dione, 2-[2-(2,6-dioxopiperidin-3-yl)-1,3-dioxo-2,3-dihydro-1H-isoindol-4-ylamino]-N-methylacetamide, 1-[2-(2,6-Dioxo-piperidin-3-yl)-1,3-dioxo-2,3-dihydro-1H-isoindol-4-ylmethyl]-3-p-tolyl-urea, or N-[2-(2,6-Dioxo-piperidin-3-yl)-1,3-dioxo-2,3-dihydro-1H-isoindol-4-ylmethyl]-2-pyridin-4-yl-acetamide. In some embodiments, a TOR kinase inhibitor is administered in combination with a compound selected from

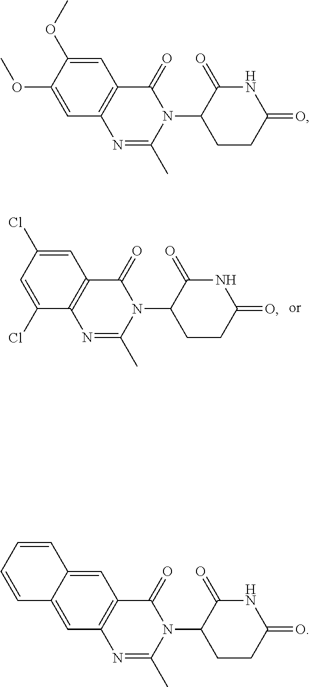 [SMILES: COc1cc2nc(C)n(C3CCC(=O)NC3=O)c(=O)c2cc1OC ; Cc1nc2c(Cl)cc(Cl)cc2c(=O)n1C1CCC(=O)NC1=O ; "or" ; Cc1nc2cc3ccccc3cc2c(=O)n1C1CCC(=O)NC1=O]

In one embodiment, the compound is:

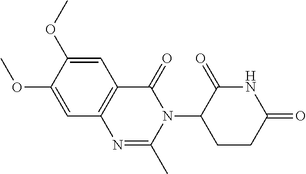 [SMILES: COc1cc2nc(C)n(C3CCC(=O)NC3=O)c(=O)c2cc1OC]

or a pharmaceutically acceptable salt, solvate, prodrug, or stereoisomer thereof. In some such embodiments, the patient is a DLBCL patient.

A TOR kinase inhibitor can be combined with radiation therapy or surgery. In certain embodiments, a TOR kinase inhibitor is administered to patient who is undergoing radiation therapy, has previously undergone radiation therapy or will be undergoing radiation therapy. In certain embodiments, a TOR kinase inhibitor is administered to a patient who has undergone tumor removal surgery.

Further provided herein are methods of reducing, treating and/or preventing adverse or undesired effects associated with conventional therapy including, but not limited to, surgery, chemotherapy, radiation therapy, hormonal therapy, biological therapy and immunotherapy. TOR kinase inhibitors and other active ingredients can be administered to a patient prior to, during, or after the occurrence of the adverse effect associated with conventional therapy.

Further provided herein are methods for treating or preventing non-small cell lung cancer (NSCLC), glioblastoma multiforme (GBM), hepatocellular carcinoma (HCC), gastrointestinal neuroendocrine tumor of non-pancreatic origin (NET), diffuse large B-cell lymphoma (DLBCL), multiple myeloma (MM), or hormone receptor positive breast cancer (HRPBC), comprising administering an effective amount of a TOR kinase inhibitor to a patient having non-small cell lung cancer (NSCLC), glioblastoma multiforme (GBM), hepatocellular carcinoma (HCC), gastrointestinal neuroendocrine tumor of non-pancreatic origin (NET), diffuse large B-cell lymphoma (DLBCL), multiple myeloma (MM) or hormone receptor positive breast cancer (HRPBC), characterized by a particular gene mutation, relative to wild type. In some embodiments described herein, the gene mutation occurs in one or more genes from Table 1, i.e. PIK3CA, RICTOR, TP53, IGF1R or PTEN. In one embodiment, the mutation is a mutation in one or more of RICTOR, TP53 or IGGF1R. In some such embodiments, a further mutation is a mutation in PIK3CA. In one embodiment, the mutation is a mutation in the gene sequence of AKT1. In one embodiment, the mutation is a gene amplication mutation in the gene sequence of AKT2. In one embodiment, the mutation is a mutation in RICTOR. In another, the mutation is a mutation in TP53. In yet another, the mutation is a mutation in IGF1R. In some such embodiments, a further mutation results in PTEN loss. In some such embodiments, the breast cancer is ER+. In some such embodiments, the breast cancer is PR+. In other embodiments, the breast cancer is ER+/PR+. In a particular embodiment, provided herein are methods for treating or preventing non-small cell lung cancer (NSCLC), comprising administering an effective amount of a TOR kinase inhibitor to a patient having non-small cell lung cancer (NSCLC) characterized by a particular gene mutation, relative to wild type, wherein the mutation is a mutation in Rictor. In a further particular embodiment, provided herein are methods for treating or preventing non-small cell lung cancer (NSCLC), comprising administering an effective amount of a TOR kinase inhibitor to a patient having non-small cell lung cancer (NSCLC) characterized by a particular gene mutation, relative to wild type, wherein the mutation is a mutation in RICTOR resulting in amplification of RICTOR.

Further provided herein are methods for treating or preventing non-small cell lung cancer (NSCLC), glioblastoma multiforme (GBM), hepatocellular carcinoma (HCC), gastrointestinal neuroendocrine tumor of non-pancreatic origin (NET), diffuse large B-cell lymphoma (DLBCL), multiple myeloma (MM), or hormone receptor positive breast cancer (HRPBC), comprising administering an effective amount of a TOR kinase inhibitor to a patient having non-small cell lung cancer (NSCLC), glioblastoma multiforme (GBM), hepatocellular carcinoma (HCC), gastrointestinal neuroendocrine tumor of non-pancreatic origin (NET), diffuse large B-cell lymphoma (DLBCL), multiple myeloma (MM) or hormone receptor positive breast cancer (HRPBC), characterized by one or more variants, relative to wild type. In some embodiments, the variant occurs in one or more genes from FIG. 2, Table 2 or Table 3. In one embodiment, the variant is one or more known somatic-variants, likely-somatic variants, rearrangements, variants-of-unknown-significance, or copy-number variants, for example, amplifications or deletions, or a combination thereof. In one embodiment, the variant is one or more known somatic variants. In another embodiment, the variant is one or more likely somatic-variants. In one embodiment, the variant is one or more rearrangements. In one embodiment, the variant is one or more variants-of-unknown-significance. In one embodiment, the variant is one or more amplifications. In another embodiment, the variant is one or more deletions.

In one embodiment, the variant is one or more known somatic variants of genes selected from AKT1, ATM, BRAF, CDKN2A, CTNNB1, ERBB2, ERBB4, ESR1, EZH2, FANCM, FBXW7, FGFR1, FGFR2, KRAS, MAP2K1, MLH1, MSH6, MTOR, PIK3CA, PTEN, TP53, TRRAP, and TSC2. In another embodiment, the variant is one or more known somatic variants of genes selected from AKT1, ATM, BRAF, CDKN2A, CTNNB1, ERBB2, ERBB4, E5R1, EZH2, FBXW7, FGFR1, FGFR2, KRAS, MAP2K1, MSH6, MTOR, PIK3CA, TP53, TRRAP, TSC2, or VHL.

In one embodiment, the variant is one or more likely somatic-variants of genes selected from APC, ARID1A, ASXL1, ATRX, BACH1, BRCA1, BRCA2, CDH1, DNMT3A, FAM123B, FLT3, IKZF1, NOTCH2, NOTCH3, PTEN, PTPRD, RB1, SMARCA4, STK11, TNFAIP3, TP53, or TSC1. In another embodiment, the variant is one or more likely somatic-variants of genes selected from APC, ARID1A, ASXL1, ATRX, BRCA1, BRCA2, CDH1, DNMT3A, FAM123B, FLT3, IKZF1, NOTCH2, NOTCH3, PTEN, PTPRD, RB1, SMARCA4, STK11, TNFAIP3, TP53, or TSC1.

In one embodiment, the variant is one or more rearrangements in genes selected from BRCA1, BRCA2, or FANCA.

In one embodiment, the variant is one or more amplifications of genes selected from BCL2L1, CCND1, CCNE1, EGFR, FGFR1, IGF1R, KDR, KIT, MCL1, MYC, MYST3, NKX2-1, PDGFRA, PIK3CA, RICTOR, SOX2, SRC, or ZNF217. In another embodiment, the variant is one or more amplifications of genes selected from BCL2L1, CCND1, CCNE1, EGFR, FGFR1, IGF1R, KDR, KIT, MCL1, MYC, MYST3, NKX2-1, PDGFRA, PIK3CA, RICTOR, or SOX2.

In one embodiment, the variant is one or more deletions in genes selected from CDKN2A, or CDKN2B. In another embodiment, the variant is one or more deletions in genes selected from CDKN2A, CDKN2B, or TSC2.

In one embodiment, the variant is one or more variants-of-unknown-significance in genes selected from ABL1, ABL2, AKT1, AKT3, ALK, APC, APCDD1, AR, ARAF, ARID1A, ASXL1, ATM, ATR, ATRX, AURKA, AURKB, AXL, BCL2, BCL6, BLM, BRAF, BRCA1, BRCA2, BRIP1, C11orf30, CARD11, CBL, CCND1, CCND3, CDC73, CDH2, CDH20, CDH5, CDK12, CDK4, CDK6, CDK8, CDKN2A, CDKN2C, CEBPA, CHEK1, CHEK2, CIC, CREBBP, CRKL, CTNNA1, CTNNB1, CUL4A, CUL4B, DAXX, DDR2, DNMT3A, DOT1L, EGFR, EPHA3, EPHA5, EPHA6, EPHA7, EPHB1, EPHB4, EPHB6, ERBB2, ERBB3, ERBB4, ERCC2, ERG, ESR1, FAM123B, FANCA, FANCM, FAT3, FBXW7, FGF12, FGF7, FGFR1, FGFR2, FGFR3, FLT1, FLT3, FLT4, FOXP4, GATA2, GNAQ, GNAS, GPR124, GRIN2A, GUCY1A2, HOXA3, HSP90AA1, IDH1, IGF1R, IGF2R, IKBKE, IKZF1, IL7R, INHBA, IRS2, JAK1, JAK2, JAK3, JUN, KDM5A, KDM5C, KDM6A, KDR, KEAP1, KIT, KLHL6, LRP1B, LRP6, MAP2K1, MAP2K2, MAP2K4, MAP3K1, MAP3K13, MCL1, MDM4, MEF2B, MEN1, MET, MITF, MLH1, MLL, MLL2, MSH2, MSH6, MTOR, MUTYH, MYCL1, MYCN, MYST3, NF1, NF2, NFE2L2, NKX2-1, NOTCH1, NOTCH2, NOTCH3, NOTCH4, NSD1, NTRK1, NTRK2, NTRK3, PAK3, PAK7, PARP1, PARP2, PARP3, PARP4, PAX5, PDGFRA, PDGFRB, PHLPP2, PIK3CA, PIK3CG, PIK3R1, PIK3R2, PKHD1, PLCG1, PNRC1, PRDM1, PRKDC, PTCH1, PTEN, PTPRD, RAD50, RAD51C, RAF1, RARA, RB1, RET, RICTOR, RPA1, RPTOR, RUNX1T1, SETD2, SF3B1, SH2B3, SMARCA4, SMO, SOX10, SPEN, SPOP, SRC, STAT3, STK11, SUFU, SYK, TBX22, TET2, TGFBR2, TIPARP, TNFAIP3, TNKS2, TOP1, TP53, TRRAP, TSC1, TSC2, TSHR, UGT1A7, USP9X, VHL, ZNF217, or ZNF703. In another embodiment, the variant is one or more variants-of-unknown-significance in genes selected from ABL1, ABL2, AKT1, AKT3, ALK, APC, APCDD1, AR, ARAF, ARID1A, ASXL1, ATM, ATR, ATRX, AURKA, AURKB, AXL, BAP1, BCL2, BCL6, BLM, BRAF, BRCA1, BRCA2, BRIP1, C11orf30, CARD11, CBL, CCND1, CCND3, CDC73, CDH2, CDH20, CDH5, CDK12, CDK6, CDK8, CDKN2A, CDKN2C, CEBPA, CEBPA, CEBPA, CEBPA, CHEK1, CHEK2, CIC, CREBBP, CRKL, CTNNA1, CTNNB1, CUL4A, CUL4B, DAXX, DDR2, DNMT3A, DOT1L, EGFR, EPHA3, EPHA5, EPHA6, EPHA7, EPHB1, EPHB4, EPHB6, ERBB2, ERBB3, ERBB4, ERCC2, ERG, ESR1, FAM123B, FANCA, FANCM, FAT3, FBXW7, FGF12, FGF7, FGFR1, FGFR2, FGFR3, FLT1, FLT3, FLT4, FOXP4, GNAQ, GNAS, GPR124, GRIN2A, GUCY1A2, HOXA3, HSP90AA1, IDH1, IGF1R, IGF2R, IKBKE, IKZF1, IL7R, INHBA, INSR, IRS2, JAK1, JAK2, JAK3, JUN, KDM5A, KDM5C, KDM6A, KDR, KEAP1, KIT, KLHL6, LRP1B, LRP6, MAP2K1, MAP2K2, MAP2K4, MAP3K1, MAP3K13, MCL1, MDM4, MEF2B, MET, MITF, MLH1, MLL, MLL2, MSH2, MSH6, MTOR, MUTYH, MYCL1, MYCL1, MYCN, MYST3, NF1, NF2, NFE2L2, NKX2-1, NOTCH1, NOTCH2, NOTCH3, NOTCH4, NSD1, NTRK1, NTRK2, NTRK3, PAK3, PAK7, PARP2, PARP3, PARP4, PAX5, PDGFRA, PDGFRB, PHLPP2, PIK3CA, PIK3CG, PIK3R1, PIK3R2, PKHD1, PLCG1, PNRC1, PRDM1, PRKDC, PTCH1, PTEN, PTPRD, RAD50, RAD51C, RAF1, RARA, RB1, RICTOR, RPA1, RPTOR, RUNX1T1, SETD2, SH2B3, SMARCA4, SMO, SOX10, SPEN, SPOP, SRC, STAT3, STK11, SUFU, SYK, TBX22, TET2, TGFBR2, TIPARP, TNFAIP3, TNKS, TNKS2, TOP1, TP53, TRRAP, TSC1, TSC2, TSHR, UGT1A7, USP9X, VHL, ZNF217, or ZNF703.

### 5.5 Pharmaceutical Compositions and Routes of Administration

Provided herein are compositions comprising an effective amount of a TOR kinase inhibitor and compositions comprising an effective amount of a TOR kinase inhibitor and a pharmaceutically acceptable carrier or vehicle. In some embodiments, the pharmaceutical composition described herein are suitable for oral, parenteral, mucosal, transdermal or topical administration.

The TOR kinase inhibitors can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the TOR kinase inhibitor in the pharmaceutical composition may be at a level that will exercise the desired effect; for example, about 0.005 mg/kg of a patient's body weight to about 10 mg/kg of a patient's body weight in unit dosage for both oral and parenteral administration.

The dose of a TOR kinase inhibitor to be administered to a patient is rather widely variable and can be patient to the judgment of a health-care practitioner. In general, the TOR kinase inhibitors can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight or about 0.25 mg/kg of a patient's body weight to about 0.5 mg/kg of a patient's body weight. In one embodiment, one dose is given per day In another embodiment, two doses are given per day. In any given case, the amount of the TOR kinase inhibitor administered will depend on such factors as the solubility of the active component, the formulation used and the route of administration.

In another embodiment, provided herein are methods for the treatment or prevention of a disease or disorder comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a TOR kinase inhibitor to a patient in need thereof. In a particular embodiment, the methods disclosed herein comprise the administration of 15 mg/day, 30 mg/day, 45 mg/day or 60 mg/day of a TOR kinase inhibitor to a patient in need thereof. In another, the methods disclosed herein comprise administration of 0.5 mg/day, 1 mg/day, 2 mg/day, 4 mg/day, 8 mg/day, 16 mg/day, 20 mg/day, 25 mg/day, 30 mg/day or 40 mg/day of a TOR kinase inhibitor to a patient in need thereof.

In another embodiment, provided herein are methods for the treatment or prevention of a disease or disorder comprising the administration of about 0.1 mg/day to about 1200 mg/day, about 1 mg/day to about 100 mg/day, about 10 mg/day to about 1200 mg/day, about 10 mg/day to about 100 mg/day, about 100 mg/day to about 1200 mg/day, about 400 mg/day to about 1200 mg/day, about 600 mg/day to about 1200 mg/day, about 400 mg/day to about 800 mg/day or about 600 mg/day to about 800 mg/day of a TOR kinase inhibitor to a patient in need thereof. In a particular embodiment, the methods disclosed herein comprise the administration of 0.1 mg/day, 0.5 mg/day, 1 mg/day, 10 mg/day, 15 mg/day, 20 mg/day, 30 mg/day, 40 mg/day, 45 mg/day, 50 mg/day, 60 mg/day, 75 mg/day, 100 mg/day, 125 mg/day, 150 mg/day, 200 mg/day, 250 mg/day, 300 mg/day, 400 mg/day, 600 mg/day or 800 mg/day of a TOR kinase inhibitor to a patient in need thereof.

In another embodiment, provided herein are unit dosage formulations that comprise between about 0.1 mg and about 2000 mg, about 1 mg and 200 mg, about 35 mg and about 1400 mg, about 125 mg and about 1000 mg, about 250 mg and about 1000 mg, or about 500 mg and about 1000 mg of a TOR kinase inhibitor.

In a particular embodiment, provided herein are unit dosage formulation comprising about 0.1 mg, 0.25 mg, 0.5 mg, 1 mg, 5 mg, 10 mg, 15 mg, 20 mg, 30 mg, 45 mg, 50 mg, 60 mg, 75 mg, 100 mg, 125 mg, 150 mg, 200 mg, 250 mg, 300 mg, 400 mg, 600 mg or 800 mg of a TOR kinase inhibitor.

In another embodiment, provided herein are unit dosage formulations that comprise 0.1 mg, 0.25 mg, 0.5 mg, 1 mg, 2.5 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a TOR kinase inhibitor. In a particular embodiment, provided herein are unit dosage formulations that comprise about 5 mg, about 15 mg, about 20 mg, about 30 mg, about 45 mg, and about 50 mg of a TOR kinase inhibitor.

A TOR kinase inhibitor can be administered once, twice, three, four or more times daily.

A TOR kinase inhibitor can be administered orally for reasons of convenience. In one embodiment, when administered orally, a TOR kinase inhibitor is administered with a meal and water. In another embodiment, the TOR kinase inhibitor is dispersed in water or juice (e.g., apple juice or orange juice) and administered orally as a suspension. In another embodiment, when administered orally, a TOR kinase inhibitor is administered in a fasted state.

The TOR kinase inhibitor can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a TOR kinase inhibitor without an additional carrier, excipient or vehicle.

In another embodiment, provided herein are compositions comprising an effective amount of a TOR kinase inhibitor and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a TOR kinase inhibitor with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a TOR kinase inhibitor as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the TOR kinase inhibitor can be delayed or prolonged by proper formulation. For example, a slowly soluble pellet of the TOR kinase inhibitor can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the TOR kinase inhibitor in oily or emulsified vehicles that allow it to disperse slowly in the serum.

### 5.6 Kits

In certain embodiments, provided herein are kits comprising a TOR kinase inhibitor. In particular embodiments, provided herein are kits comprising a unit dosage form comprising a TOR kinase inhibitor in a sealed container, wherein the unit dosage form comprises about 1 mg to about 100 mg of a TOR kinase inhibitor. In particular embodiments, provided herein are kits comprising a unit dosage form comprising a TOR kinase inhibitor in a sealed container, wherein the unit dosage form comprises about 5 mg, about 20 mg or about 50 mg of a TOR kinase inhibitor.

In other embodiments, provide herein are kits comprising a TOR kinase inhibitor and means for monitoring patient response to administration of said TOR kinase inhibitor. In certain embodiments, the patient has a cancer, for example breast cancer characterized by a gene mutation, for example a mutation in one or more genes from Table 1. In certain embodiments, the patient has a cancer, for example breast cancer, DLBCL, GBM, HCC, MM, NET, or NSCLC, characterized by one or more gene variants, for example a variant in one or more genes from FIG. 2. In certain embodiments, the patient has a cancer, for example breast cancer, DLBCL, GBM, HCC, MM, NET, or NSCLC, characterized by one or more gene variants, for example a variant in one or more genes from Table 2 or Table 3. In certain embodiments, the patient has a cancer, for example breast cancer, DLBCL, GBM, HCC, MM, NET, or NSCLC, characterized by one or more gene variants, for example a variant in one or more genes as described herein. In particular embodiments, the patient response measured is inhibition of disease progression, inhibition of tumor growth, reduction of primary and/or secondary tumor(s), relief of tumor-related symptoms, improvement in quality of life, inhibition of tumor secreted factors (including tumor secreted hormones, such as those that contribute to carcinoid syndrome), delayed appearance of primary and/or secondary tumor(s), slowed development of primary and/or secondary tumor(s), decreased occurrence of primary and/or secondary tumor(s), slowed or decreased severity of secondary effects of disease, arrested tumor growth and/or regression of tumors.

In other embodiments, provide herein are kits comprising a TOR kinase inhibitor and means for monitoring patient response to administration of said TOR kinase inhibitor, wherein said response is Response Evaluation Criteria in Solid Tumors (for example, RECIST 1.1) or Eastern Cooperative Oncology Group Performance Status (ECOG).

In other embodiments, provided herein are kits comprising a TOR kinase inhibitor and means for measuring the amount of inhibition of phosphorylation of S6RP, 4E-BP1 and/or AKT in a patient. In certain embodiments, the kits comprise means for measuring inhibition of phosphorylation of S6RP, 4E-BP1 and/or AKT in circulating blood or tumor cells and/or skin biopsies or tumor biopsies/aspirates of a patient. In certain embodiments, provided herein are kits comprising a TOR kinase inhibitor and means for measuring the amount of inhibition of phosphorylation as assessed by comparison of the amount of phospho-S6RP, 4E-BP1 and/or AKT before, during and/or after administration of the TOR kinase inhibitor. In certain embodiments, the patient has a cancer, for example breast cancer characterized by a gene mutation, for example a mutation in one or more genes from Table 1. In certain embodiments, the patient has a cancer, for example breast cancer, DLBCL, GBM, HCC, MM, NET, or NSCLC, characterized by one or more gene variants, for example a variant in one or more genes from FIG. 2. In certain embodiments, the patient has a cancer, for example breast cancer, DLBCL, GBM, HCC, MM, NET, or NSCLC, characterized by one or more gene variants, for example a variant in one or more genes from Table 2 or Table 3. In certain embodiments, the patient has a cancer, for example breast cancer, DLBCL, GBM, HCC, MM, NET, or NSCLC, characterized by one or more gene variants, for example a variant in one or more genes as described herein.

In other embodiments, provided herein are kits comprising a TOR kinase inhibitor and means for measuring the amount of inhibition of DNA-dependent protein kinase (DNA-PK) activity in a patient. In certain embodiments, the kits comprise means for measuring the amount of inhibition of DNA-dependent protein kinase (DNA-PK) activity in a skin sample and/or a tumor biopsy/aspirate of a patient. In one embodiment, the kits comprise a means for measuring the amount of pDNA-PK S2056 in a skin sample and/or a tumor biopsy/aspirate of a patient. In one embodiment, the skin sample is irradiated by UV light. In certain embodiments, provided herein are kits comprising a TOR kinase inhibitor and means for measuring the amount of inhibition of DNA-dependent protein kinase (DNA-PK) activity before, during and/or after administration of the TOR kinase inhibitor. In certain embodiments, provided herein are kits comprising a TOR kinase inhibitor and means for measuring the amount of phosphorylated DNA-PK S2056 before, during and/or after administration of the TOR kinase inhibitor. In certain embodiments, the patient has a cancer, for example breast cancer characterized by a gene mutation, for example a mutation in one or more genes from Table 1. In certain embodiments, the patient has a cancer, for example breast cancer, DLBCL, GBM, HCC, MM, NET, or NSCLC, characterized by one or more gene variants, for example a variant in one or more genes from FIG. 2. In certain embodiments, the patient has a cancer, for example breast cancer, DLBCL, GBM, HCC, MM, NET, or NSCLC, characterized by one or more gene variants, for example a variant in one or more genes from Table 2 or Table 3. In certain embodiments, the patient has a cancer, for example breast cancer, DLBCL, GBM, HCC, MM, NET, or NSCLC, characterized by one or more gene variants, for example a variant in one or more genes as described herein.

Inhibition of phosphorylation of S6RP, 4E-BP1, and/or AKT can be measured in blood, skin, tumor, and/or circulating tumor cells (CTCs) in blood by various methodology including flow cytometry, ELISA, immunohistochemistry (IHC) using phosphorylation-specific antibodies. Inhibition of DNA-PK activity can be measured in blood, skin, and/or circulating tumor cells (CTCs) in blood by monitoring phosphorylation of substrates of DNA-PK, such as DNA-PK itself and XRCC4. Inhibition of DNA-PK activity can also be measured by monitoring accumulation of double strand DNA damage in tissues and/or cells such as those mentioned above.

In certain embodiments, the kits provided herein comprise an amount of a TOR kinase inhibitor effective for treating or preventing a cancer, for example breast cancer characterized by a gene mutation, for example a mutation in one or more genes from Table 1. In certain embodiments, the kits provided herein comprise an amount of a TOR kinase inhibitor effective for treating or preventing a cancer, for example breast cancer, DLBCL, GBM, HCC, MM, NET, or NSCLC, characterized by one or more gene variants, for example a variant in one or more genes from FIG. 2. In certain embodiments, the kits provided herein comprise an amount of a TOR kinase inhibitor effective for treating or preventing a cancer, for example breast cancer, DLBCL, GBM, HCC, MM, NET, or NSCLC, characterized by one or more gene variants, for example a variant in one or more genes from Table 2 or Table 3. In certain embodiments, the kits provided herein comprise an amount of a TOR kinase inhibitor effective for treating or preventing a cancer, for example breast cancer, DLBCL, GBM, HCC, MM, NET, or NSCLC, characterized by one or more gene variants, for example a variant in one or more genes as described herein. In certain embodiments, the kits provided herein comprise a TOR kinase inhibitor having the molecular formula C₁₆H₁₆N₈O. In certain embodiments, the kits provided herein comprise Compound 1.

In certain embodiments, the kits provided herein further comprise instructions for use, such as for administering a TOR kinase inhibitor and/or monitoring patient response to administration of a TOR kinase inhibitor.

### 6. EXAMPLES

### 6.1 Biochemical Assays

mTOR HTR-FRET Assay.

The following is an example of an assay that can be used to determine the TOR kinase inhibitory activity of a test compound. TOR kinase inhibitors were dissolved in DMSO and prepared as 10 mM stocks and diluted appropriately for the experiments. Reagents were prepared as follows:

“Simple TOR buffer” (used to dilute high glycerol TOR fraction): 10 mM Tris pH 7.4, 100 mM NaCl, 0.1% Tween-20, 1 mM DTT. Invitrogen mTOR (cat#PV4753) was diluted in this buffer to an assay concentration of 0.200 μg/mL.

ATP/Substrate solution: 0.075 mM ATP, 12.5 mM MnCl₂, 50 mM Hepes, pH 7.4, 50 mM β-GOP, 250 nM Microcystin LR, 0.25 mM EDTA, 5 mM DTT, and 3.5 μg/mL GST-p70S6.

Detection reagent solution: 50 mM HEPES, pH 7.4, 0.01% Triton X-100, 0.01% BSA, 0.1 mM EDTA, 12.7 μg/mL Cy5-αGST Amersham (Cat#PA92002V), 9 ng/mL α-phospho p70S6 (Thr389) (Cell Signaling Mouse Monoclonal #9206L), 627 ng/mL α-mouse Lance Eu (Perkin Elmer Cat#AD0077).

To 20 μL of the Simple TOR buffer is added 0.5 μL of test compound in DMSO. To initiate the reaction 5 μL of ATP/Substrate solution was added to 20 μL of the Simple TOR buffer solution (control) and to the compound solution prepared above. The assay was stopped after 60 min by adding 5 μL of a 60 mM EDTA solution; 10 μL of detection reagent solution was then added and the mixture was allowed to sit for at least 2 hours before reading on a Perkin-Elmer Envision Microplate Reader set to detect LANCE Eu TR-FRET (excitation at 320 nm and emission at 495/520 nm).

TOR kinase inhibitors were tested in the mTor HTR-FRET assay and were found to have activity therein, with certain compounds having an IC₅₀ below 10 μM in the assay, with some compounds having an IC₅₀ between and 0.005 nM and 250 nM, others having an IC₅₀ between and 250 nM and 500 nM, others having an IC₅₀ between 500 nM and 1 μM, and others having an IC₅₀ between 1 μM and 10 μM.

DNA-PK assay. DNA-PK assay is performed using the procedures supplied in the Promega DNA-PK assay kit (catalog # V7870). DNA-PK enzyme can be purchased from Promega (Promega cat#V5811).

Selected TOR kinase inhibitors as described herein have, or are expected to have, an IC₅₀ below 10 μM in this assay, with some TOR kinase inhibitors as described herein having an IC₅₀ below 1 μM, and others having an IC₅₀ below 0.10 μM.

### 6.2 Clinical Study A

A Phase 1/2, Multi-Center, Open-Label, Dose Finding Study to Assess the Safety, Tolerability, Pharmacokinetics and Preliminary Efficacy of Compound 1 Administered Orally to Subjects with Advanced Solid Tumors, Non-Hodgkin Lymphoma or Multiple Myeloma.

Compound 1 will be administered orally to subjects with solid tumors, non-Hodgkin lymphoma or multiple myeloma. The study is designed as a Phase 1/2 trial consisting of two parts: dose escalation (Part A) and dose expansion (Part B).

Compound 1 will be administered orally to determine safety and tolerability and to define the non-tolerated dose (NTD) and the maximum tolerated dose (MTD).

Evaluations will include the extent of inhibition of phosphorylation of S6RP (Ser235/236 and/or Ser240/244) and/or 4EB-P1 (Thr37/46) for mTORC1 activity and AKT (Ser473) and/or other relevant biomarkers for mTORC2 activity in peripheral blood samples and tumor biopsies following treatment with Compound 1, and the efficacy of Compound 1.

The study population will consist of men and women, 18 years or older, with advanced NHL, MM, neuroendocrine tumors (the latter also accepting subjects aged 12 years or older) or advanced unresectable solid tumors, including subjects who have progressed on (or not been able to tolerate) standard therapy or for whom no standard anticancer therapy exists.

For both the dose escalation and dose expansion parts of this protocol, inclusion criteria are: (1) Understand and voluntarily sign an informed consent document prior to any study related assessments/procedures are conducted; (2) Men and women, 18 years or older, with histologically or cytologically-confirmed, advanced NHL, MM, or advanced unresectable solid tumors including subjects who have progressed on (or not been able to tolerate) standard anticancer therapy or for whom no standard anticancer therapy exists; (3) Eastern Cooperative Oncology Group Performance Status (ECOG) PS of 0 or 1 for subjects with solid tumors, and 0-2 for hematologic malignancies; (4) Subjects must have the following laboratory values: Absolute Neutrophil Count (ANC)≧1.5×10⁹/L, Hemoglobin (Hgb)≧9 g/dl, Platelets (plt)≧100×10⁹/L, Potassium within normal limits or correctable with supplements, AST/SGOT and ALT/SGPT≦2.5× Upper Limit of Normal (ULN) or ≦5.0×ULN if liver tumor is present, Serum bilirubin ≦1.5×ULN or ≦2×ULN if liver tumor is present, Serum creatinine ≦1.5×ULN or 24-hour clearance ≧50 mL/min, Negative serum or urine pregnancy test within 48 hours before starting study treatment in females of childbearing potential; and (5) Able to adhere to the study visit schedule and other protocol requirements

For the dose expansion part (Part B) of this protocol, inclusion criteria are: (1) Retrieval of formalin-fixed, paraffin embedded (FFPE) archival tumor tissue, either in tumor blocks or sectioned/mounted specimens for gene mutation and/or IHC biomarker assay for all tumors except MM. Only in exceptional circumstances may an exemption waiver be granted by the Sponsor for other tumor types; (2) Satisfactory Screening biopsy for gene mutation and/or IHC biomarker assay for accessible tumors for all tumors except NSCLC and NET (optional) and GBM; (3) Histologically-confirmed tumors of the following types, all with measurable disease. Type-specific criteria are in addition to, or supersede, above criteria where applicable: (a) Glioblastoma multiforme (GBM) or gliosarcoma, excluding WHO Grade IV oligoastrocytoma (has received prior treatment including radiation and/or chemotherapy, with radiation completed >12 weeks prior to Day 1; planned salvage surgical tumor resection on Day 15±7 days, anticipated to yield ≧200 mg tumor tissue; no prior or scheduled Gliadel® wafer implant unless area of assessment and planned resection is outside the region previously implanted; no prior interstitial brachytherapy or stereotactic radiosurgery unless area of assessment and planned resection is outside the region previously treated; no enzyme-inducing anti-epileptic drugs (EIAED) such as carbamazepine, phenytoin, phenobarbital, or primidone within 14 days before Day 1; able to undergo repeated magnetic resonance imaging (MRI) scans; Availability of adequate FFPE archival tumor material (for PD biomarkers)); (b) Hepatocellular carcinoma (HCC) (Plt count ≧60×10⁹/L if portal hypertension is present; Child-Pugh score of less than 10 (i.e., class B liver function or better); at least 4 weeks from last dose of α-interferon and/or ribivirin; at least 4 weeks from prior percutaneous ethanol injection, radiofrequency ablation, transarterial embolization, or cryotherapy with documentation of progressive or recurrent disease); (c) Gastrointestinal neuroendocrine tumor (NET) of non-pancreatic origin (locally unresectable or metastatic differentiated, low (grade 1) or intermediate (grade 2), non-pancreatic NET or NET of unknown primary origin; pancreaticpheochromocytomas, paragangliomas, adenocarcinoid and goblet carcinoid tumors, and poorly differentiated, high grade (eg., small cell or large cell) tumors are excluded; subjects aged 12 years or older; symptomatic endocrine-producing tumors and nonfunctional tumors are both allowed; agreement to concurrent therapy with somatostatin analogs; evidence of radiologic disease progression within 12 months prior to Cycle 1, Day 1; no receptor targeted radiolabeled therapy within 3 months prior to Cycle 1, Day 1; no liver-directed therapy within 4 weeks prior to Cycle 1, Day 1, unless a site of measureable disease other than the treated lesion is present; screening and on-study tumor biopsies are optional in this cohort; archival tumor collection should be requested, but is not mandatory in this cohort); (d) Hormone receptor-positive breast cancer (HRPBC) (unresectable locally advanced or metastatic carcinoma of the breast; ER positive, and HER2/neu negative (0 or 1+), tumor; measurable disease according to RECIST v1.1; must have received at least one prior line of hormonal therapy or at least one year of aromatase therapy in the adjuvant setting, or six months of aromatase inhibitor therapy for metastatic disease; bisphosphonates or denusomab are allowed in stable doses; cohort may be expanded to enroll a minimum of 5 subjects each with tumors containing PIK3CA mutations; (e) Multiple Myeloma (MM) (measurable levels of myeloma paraprotein in serum (>0.5 g/dL) or urine (>0.2 g excreted in a 24-hour collection sample); absolute neutrophil count (ANC)≧1.0×10⁹/L; platelets (plt)≧60×10⁹/L in subjects in whom ≦50% of bone marrow mononuclear cells are plasma cells or ≧30×10⁹/L in subjects in whom ≧50% of bone marrow mononuclear cells are plasma cells); (f) Diffuse large B-cell lymphoma (DLBCL) (histologically proven diffuse large B-cell non-Hodgkin's lymphoma; platelets (plt)≧60×10⁹/L for subjects in whom ≦50% of bone marrow mononuclear cells are lymphoma cells, or ≧30×10⁹/L for subjects in whom ≧50% of bone marrow mononuclear cells are lymphoma cells; at least 4 weeks from last dose of therapeutic glucocorticosteroids; adrenal replacement doses of glucocorticosteroids (up to the equivalent of 10 mg daily prednisone) are allowed).

For both the dose escalation and dose expansion parts of this protocol, exclusion criteria are: (1) Symptomatic central nervous system metastases (excluding GBM; subjects with brain metastases that have been previously treated and are stable for 6 weeks are allowed); (2) Known acute or chronic pancreatitis; (3) Subjects with any peripheral neuropathy ≧NCI CTCAE grade 2; (4) Subjects with persistent diarrhea or malabsorption ≧NCI CTCAE grade 2, despite medical management; (5) Impaired cardiac function or clinically significant cardiac diseases, including any of the following: LVEF<45% as determined by MUGA scan or ECHO, Complete left bundle branch, or bifasicular, block, Congenital long QT syndrome, Persistent or clinically meaningful ventricular arrhythmias or atrial fibrillation, QTcF>460 msec on screening ECG (mean of triplicate recordings), Unstable angina pectoris or myocardial infarction ≦3 months prior to starting Compound 1, Other clinically significant heart disease such as congestive heart failure requiring treatment or uncontrolled hypertension (blood pressure ≧160/95 mmHg); (6) Subjects with diabetes on active treatment or subjects with either of the following: (a) fasting blood glucose ≧126 mg/dL (7.0 mmol/L), or (b) HbA1c≧6.5%; (7) Other concurrent severe and/or uncontrolled concomitant medical conditions (e.g., active or uncontrolled infection) that could cause unacceptable safety risks or compromise compliance with the protocol; (8) Prior systemic cancer-directed treatments or investigational modalities ≦5 half lives or 4 weeks, whichever is shorter, prior to starting study drug or who have not recovered from side effects of such therapy (subjects must have recovered from any effects of recent radiotherapy that might confound the safety evaluation of study drug); (9) Subjects who have undergone major surgery ≦2 weeks prior to starting study drug or who have not recovered from side effects of such therapy; (10) Women who are pregnant or breast feeding; Adults of reproductive potential not employing two forms of birth control: (a) females of childbearing potential must agree to use two adequate forms of contraception methods simultaneously (one must be non-hormonal) from the time of giving informed consent until 28 days after the last dose of Compound 1. Females of child-bearing potential, defined as sexually mature women who have not undergone a hysterectomy or bilateral oophorectomy, or who have not been naturally postmenopausal (ie., who have not menstruated at all) for at least 24 consecutive months; (b) males (with partners who are female with child-bearing potential must agree that they or their partners will use at least two effective contraceptive methods (including one barrier method) when engaging in reproductive sexual activity throughout the study, and will avoid conceiving for 28 days after taking the last dose of Compound 1; (11) Subjects with known HIV infection; (12) Known chronic hepatitis B or C virus (HBV/HCV) infection, unless comorbidity in subjects with HCC; (13) Any significant medical condition, laboratory abnormality, or psychiatric illness that would prevent the subject from participating in the study; (14) Any condition including the presence of laboratory abnormalities, which places the subject at unacceptable risk if he/she were to participate in the study; (15) Any condition that confounds the ability to interpret data from the study.

For the dose expansion part (Part B) of this protocol, exclusion criteria are: (1) Concurrent active second malignancy for which the patient is receiving therapy, excluding non-melanomatous skin cancer or carcinoma in situ of the cervix.

Compound 1 will be supplied in appropriate strengths (e.g., 2.5 mg, 10 mg, and 20 mg) containing only the active pharmaceutical ingredient in reddish-brown gelatin capsules for oral administration. No other excipients will be used in the product capsules.

Compound 1 will be administered orally, in an uninterrupted once-daily schedule with no rest period between cycles. A dose of 7.5 mg/day of Compound 1 will be the starting dose in this protocol. Each dose will be taken in the morning. On clinic visit days, Compound 1 will be administered in the clinic after any predose tests have been completed. Food will be taken after all fasting tests have been completed (3 hours after dosing on Day 8). In cases where troublesome GI symptoms, fatigue or other symptoms persist beyond the end of Cycle 1, dosing may be moved to the end of day. Compound 1 may be taken up to 12 hours late if dosing has been delayed on a single day; otherwise that day's dose should be omitted.

In Part A, subjects will receive single and multiple ascending dose levels of Compound 1 to measure pharmacokinetics (PK) and to identify the maximum tolerated dose (MTD). A modified accelerated titration design (Simon R, Freidlin B, Rubinstein L, et al. Accelerated Titration Designs for Phase I Clinical Trials in Oncology, Journal of the National Cancer Institute, (1997) Vol. 89, No. 15) will be used to establish initial toxicity. During the accelerated course, initial cohorts of one subject will be given Compound 1 at dose increments of 100% until the first instance of first-course grade 2 or higher toxicity, at which point the accelerated part will be terminated, and this particular cohort will be expanded to 6 subjects. Subsequently, a standard escalation dosing schedule with approximately 50% dose increments and 6 subjects per cohort will be initiated in order to establish the non-tolerated dose (NTD) and MTD. Smaller increments and additional subjects within a dose cohort may also be evaluated.

A dose will be considered to be non-tolerated if 2 evaluable subjects in a dose cohort experience dose-limiting toxicity (DLT). When a NTD is defined, dose escalation will be stopped. The MTD will be defined as the last dose tested below the NTD with 0 or 1 out of 6 evaluable subjects experiencing DLT during Cycle 1. An intermediate dose (i.e., one between the NTD and the last dose level before the NTD) or additional subjects within any dose cohort may be required to determine the MTD more precisely.

In Part B, subjects may start Compound 1 at the MTD and/or a lower dose level based on safety, PK and PD data from Part A. Approximately 150 subjects will be treated and evaluated for safety and preliminary antitumor activity after every two cycles of therapy. Tumor types include non-small cell lung cancer (NSCLC), glioblastoma multiforme (GBM), hepatocellular carcinoma (HCC), gastrointestinal neuroendocrine tumor of non-pancreatic origin (NET), diffuse large B-cell lymphoma (DLBCL), multiple myeloma (MM), and hormone receptor positive breast cancer (HRPBC). Up to 20 subjects will be enrolled in each tumor type.

During the first cycle only in Part A, each subject will be administered a single dose of Compound 1 (Day −1), followed by a 48-hour observation and PK sampling period, followed on Day 1 by daily uninterrupted dosing for 28 days (Cycle 1=30 days). In subsequent Part A cycles, subjects are treated in 28-day cycles with continuous dosing from Day 1 to 28. In Part B, subjects will receive continuous dosing for 28 days from the beginning—there is neither an initial observation period nor a 48-hour PK collection.

Therapy may be discontinued if there is evidence of disease progression, but subjects can continue to receive Compound 1 as long as the Investigator considers they are deriving benefit from treatment. Therapy will be discontinued if there is unacceptable toxicity or if the subject decides to withdraw from the study.

When a dose reduction is indicated, the next lower dose level will be selected. Two dose reductions are allowed. For the starting dose level (7.5 mg) in Part A, reductions will be in 2.5 mg decrements. In Part B, for subjects starting at 45 mg QD dose reductions to 30 mg and 15 mg QD are permitted; for those starting at 30 mg QD, the dose reductions are 15 mg QD and 7.5 mg QD. If any subject continues to experience unacceptable toxicity after 2 dose reductions in Part A, Compound 1 will be discontinued permanently. In Part B, subjects may dose reduce up to 2 levels and increase again if clinically appropriate; subsequent dose reductions are permitted in the event of recurrent toxicity but, in such circumstances, it is not permitted to reescalate the dose again. For subjects in Part B starting at 30 mg QD, dose escalation to 45 mg QD is not allowed.

Subjects will be evaluated for efficacy every 2 cycles through cycle 6 and every 3 cycles thereafter. The primary efficacy variable is response. Tumor assessments, including imaging (CT, MRI and/or PET) of the chest and abdomen and other sites as appropriate, will be performed during Screening. Subjects with brain lesions will also have brain scans at Screening and during follow-up tumor assessments. After Screening, tumor assessments (for all tumors except multiple myeloma) will be performed on completion of Cycles 2, 4 and 6 (i.e., on Cycles 3, 5 and 7/Day 1±7 days) and then every 3 months thereafter (e.g., Cycle 10 and 13/Day 1±7 days). Tumor assessment (for multiple myeloma and only NHL/DLBCL with known or suspected marrow involvement) (bone marrow aspiration and biopsy, with PD biomarker analysis, cytogenetic analysis if abnormally present at Screening) will be performed on completion of Cycles 4, 8, 12 and 16 only (i.e., on Cycles 5, 9, 13 and 17/Day 1±7 days). Cytogenetics need not be repeated if normal at Screening. Tumor response will be based on Response Evaluation Criteria in Solid Tumors (RECIST 1.1), International Workshop Criteria (IWG) for NHL/DLBCL or International Uniform Response Criteria (IURC) for Multiple Myeloma, and RANO for GBM, using the post resection MRI scan as the baseline. Given the difficulty in assessing tumor response following salvage surgery, the primary efficacy endpoint for GBM will be the proportion of subjects progression-free at 6 months from Day 1 relative to efficacy evaluable subjects in the GBM type. Subjects will be evaluated for tumor response on completion of Cycle 2, 4, 6, and so on. A descriptive analysis of evidence of anti-tumor activity will be provided based on clinical and radiographic assessments by the investigator, which includes assessment of target lesion, non-target lesion, new lesion and overall response.

The efficacy variable of focus for Part A will be best overall response. Other preliminary efficacy variables will be summarized using frequency tabulations for categorical variables or descriptive statistics for continuous variables.

For Part B, efficacy variables to be analyzed include tumor response at the end of treatment, the proportion of subject alive and progression-free, and duration of response. Efficacy variables will mature when last subject of a treatment arm or cohort have withdrawn from the study or completed 6 cycles.

Progression Free Survival rates will be computed using the Kaplan-Meier estimates. Duration of response will also be reported in subjects who respond, using tumor specific evaluation criteria. Two-sided 90% CIs of the Response Rate (RR), Disease Control Rate (DCR) and of the Progression Free Survival (PFS) rate at time of each scheduled response assessment (ie., Cycles 2, 4, 6, etc.) will be provided by tumor type.

Other preliminary efficacy variables, including ECOG performance status, PET, carcinoid/NET-specific symptom outcomes, etc., will be summarized using frequency tabulations for categorical variables or descriptive statistics for continuous variables.

Parameters to be explored include mTOR biomarker inhibition in blood and tumor, histopathologic response, correlations with pharmacogenomic findings and percentage of inhibition of pAKT (Ser473), phospho-S6RP (Ser235/236 and/or Ser240/244), phospho-4EB-P1 (Thr37/46), and/or other relevant biomarkers in peripheral blood samples and tumor, adverse events and clinical outcome. The pharmacodynamic (PD) measurements are incorporated in this study to evaluate target inhibition of mTORC1 and mTORC2 pathways, the consequences of such inhibition, and PK/PD relationships. In Parts A and B, biomarker analysis will involve measuring pAKT (mTORC2) in protein lysates derived from isolated platelets. Levels of p4EB-P1 and pS6RP (mTORC1), and pAKT (mTORC2), will be measured by flow cytometry using whole blood samples. Likewise, in Parts A and B, pAKT, p4EB-P1, pS6, Ki67 and/or other relevant markers to assess Compound 1 activity will be measured in serial tumor biopsies from subjects with accessible disease when possible. The changes of each biomarker will be determined by comparing the levels of biomarkers in pre- and post-treatment samples and, where possible, correlate these with drug exposure in blood, and tissue if available, and tumor response over time. Full details of all statistical analyses and modeling for these outcomes will be described in the statistical analysis plan and final study report.

The safety variables for this study are adverse events, clinical laboratory variables, 12-lead ECGs (centrally reviewed), LVEF assessments, physical examinations and vital signs. In Part A, the decision to either evaluate a higher dose level or declare a MTD will be determined by the Safety Review Committee (SRC) each time all clinical and laboratory safety data for a given cohort is available for review. The SRC will also determine the dose, doses, or schedule appropriate for Part B. During Part B, the SRC will continue to review safety data regularly and make recommendations about the study continuation, as appropriate.

In certain embodiments, patients undergoing the clinical protocol provide herein will show a positive tumor response, such as inhibition of tumor growth or a reduction in tumor size. In certain embodiments, patients undergoing the clinical protocol provide herein will show an improvement in brain lesions, such as a decrease in number or size. In certain embodiments, patients undergoing the clinical protocol provide herein will achieve a Response Evaluation Criteria in Solid Tumors (for example, RECIST 1.1) of complete response, partial response or stable disease. In certain embodiments, patients undergoing the clinical protocol provided herein will prevent a Response Evaluation Criteria in Solid Tumors (RECIST 1.1) of progressive disease. In certain embodiments, patients undergoing the clinical protocol provide herein will show an improvement in International Workshop Criteria (IWC) or International Uniform Response Criteria (IURC). In certain embodiments, patients undergoing the clinical protocol provide herein will show an improvement in Response Assessment for Neuro-Oncology (RANO) Working Group criteria. In certain embodiments, patients undergoing the clinical protocol provide herein will show an improvement in ECOG performance status or PET outcomes. In certain embodiments, patients undergoing the clinical protocol provide herein will show a reduction in a carcinoid syndrome-related symptom, for example, one or more of flushing, diarrhea, joint pain, bone pain, colicky abdominal pain, fatigue, wheezing, rash, cough, shortness of breath, edema or hypertension.

TOR Pathway Biomarker Measurements in Whole Blood.

Blood samples received from clinical sites were aliquoted into a 96-deepwell plate and rested for 1 hour at 37° C. The samples were stimulated with anti-IgD and LPS for 15 minutes at 37° C. The red blood cells were lysed and the white blood cells were fixed with BD Lyse/Fix Buffer at a ratio of 15:1 buffer to blood for 10 minutes at 37° C. The plates were centrifuged, aspirated, and 1 mL of ice-cold methanol was added to the wells containing fixed white blood cells to permeabilize the cells for intracellular staining. The plates were stored overnight at −80° C. The plates were thawed, centrifuged, aspirated and washed twice with PBS+0.5% BSA. The cells were stained with antibodies specific for the surface markers CD3, CD14, and CD19, and for mTOR pathway markers, including pS6 (S235/236), p4EBP1 (T37/46), and pAKT (S473). The cells were washed twice with PBS and fixed with 1.6% PFA.

Sample analysis: The samples were analyzed on an 8 color cytometer. Control wells of 8-peak rainbow beads (Spherotech Libertyville, Ill.) were acquired at multiple points during sample acquisition. The median fluorescence intensity (MFI) was computed for each marker from the fluorescence intensity levels in T cells, B cells, and monocytes. The MFI were normalized using the 8-peak rainbow beads and presented as ERF (Equivalent number of Reference Fluorophores). ERFs were calculated from the MFIs using a linear regression transformation carried out on a log-log scale using the rainbow calibration particles with 8 intensities on 8 colors. The percent change from baseline for pS6, p4EBP1, and pAKT in stimulated and non-stimulated T cells, B cells, and monocytes was determined for each patient. The baseline value was an average of two visits (screening and cycle 1/day −1 at 0 hr pre-dose) when available.

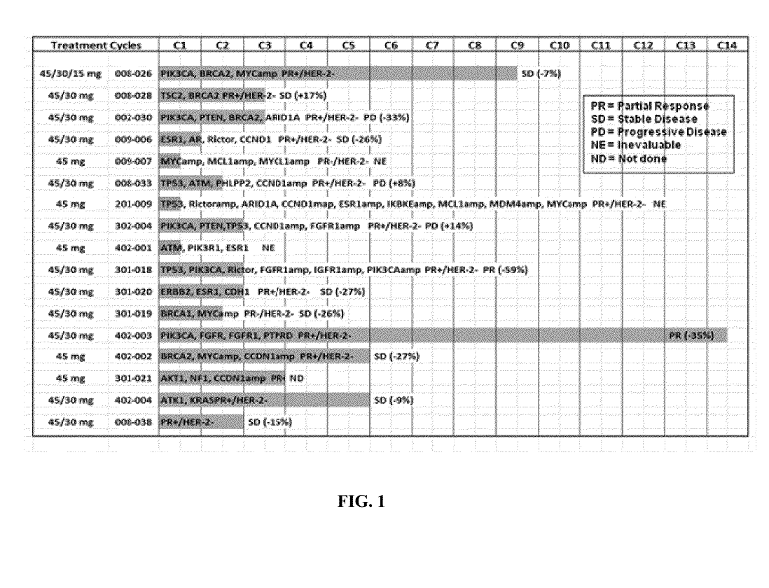

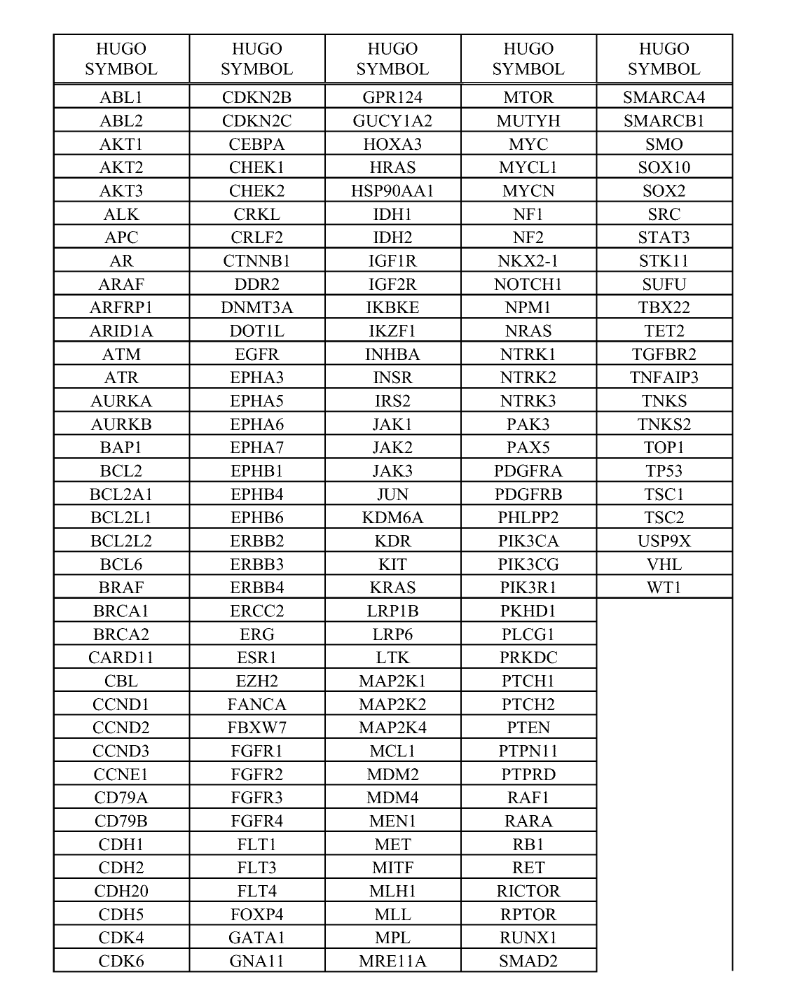

As can be seen in FIG. 1 (data as of September 2014), signals of Compound 1 clinical activity were demonstrated with 3/17 showing target lesion PR (2/17 showing RECIST PR), all with PIK3CA mutations, in addition to mutations in RICTOR, TP53, IGF1R and/or PTEN. Additionally, mutations in BRCA2, ARID1A, FGFR1, FGFR and PTPRD were observed.

### 6.3 Mutational Analysis

DNA Extraction

Selected frozen or fixed tissue and tumor content was enriched to an estimated 50% in the selected frozenblock, sections of 20 μm were cut in a cryostat and were disrupted and homogenized chemically (added in RLT plus buffer (Qiagen, Courtaboeuf, France) with β-mercaptoethanol (Sigma Aldrich, Saint-Quentin Fallavier, France). The disruption was finalized mechanically, in ice, with a Rotor-stator homogenizer (Kimble Chase Scientific, Vineland, N.J.). The extraction was performed with the AllPrep DNA/RNA Mini Kit (Qiagen) for simultaneous purification of genomic DNA and total RNA from the same tissue sample. DNA was quantified by spectrophotometry with NanoDrop 1000 (Thermo Scientific, Waltham, Mass.). DNA was qualified by agarose gel electrophoresis bioanalyzer (Agilent, Santa Clara, Calif.). Fixed tissues were enriched to ≧25% tumor in 4-5 micron thick sections on glass slides, deparafinized and extracted using the Maxwell magnetic bead platform (Promega, WI).

NGS DNA Library Construction and Hybrid Capture.

Molecular barcode-indexed, ligation-based sequencing libraries were constructed by using 200 ng of sheared DNA or total DNA recovered from the sample (if ≧50 ng) when 200 ng was not available. Libraries were hybridization captured with custom biotinylated RNA oligo pools (custom SureSelect kit, Agilent) representing 3,230 exons in 182 cancer-related genes plus 37 introns from 14 genes often rearranged in cancer (189 genes total, seven genes were screened across both exons and introns).

Sequencing and Analysis.

Paired-end sequencing (49×49 cycles) was performed by using the HiSeq2000 (Illumina, San Diego, Calif.) in a Clinical Laboratory Improvement Amendments (CLIA) laboratory (Foundation Medicine). Sequence data from genomic DNA was mapped to the reference human genome (hg19) by using the Burrows-Wheeler Aligner (BWA) (see Li H, Durbin R, Fast and accurate short read alignment with Burrows-Wheeler transform. Bioinformatics 25:1754-1760, 2009) and was processed by using publicly available SAMtools (see Li H, Handsaker B, Wysoker A, et al: The Sequence Alignment/Map format and SAMtools. Bioinformatics 25:2078-2079, 2009), Picard (http://picard.sourceforge.net) and the Genome Analysis Toolkit (see McKenna A, Hanna M, Banks E, et al: The Genome Analysis Toolkit: A MapReduce framework for analyzing next-generation DNA sequencing data, Genome Res 20:1297-1303, 2010). Genomic base substitutions and indels were detected by using custom tools optimized for mutation calling in heterogeneous tumor samples on the basis of statistical modeling of sequence quality scores and local sequence assembly. Variations were filtered by using dbSNP_{—}135 (http://www.ncbi.nlm.nih.gov/projects/SNP/) and a custom artifact database (Foundation Medicine, artifact databases 2011 through 2013) and were then annotated for known and likely somatic mutations by using COSMIC (see Forbes S A, Bindal N, Bamford S, et al: COSMIC: Mining complete cancer genomes in the Catalogue of Somatic Mutations in Cancer. Nucleic Acids Res 39:D945-D950, 2011). Copy number alterations were detected by comparing targeted genomic DNA sequence coverage with a process-matched normal control sample. Genomic rearrangements were detected by clustering chimeric reads mapping to targeted introns. To maximize mutation-detection sensitivity, the test was validated to detect base substitutions at a ≧10% mutant allele frequency with ≧99% sensitivity and to detect indels at a ≧20% mutant allele frequency with ≧95% sensitivity, with a false discovery rate of less than 1%. Recurrent somatic alterations were defined as genomic alterations in genes that are mutated ≧5% in COSMIC, or amplified or deleted at ≧5% in the literature (see Ding L, Getz G, Wheeler D A, et al: Somatic mutations affect key pathways in lung adenocarcinoma. Nature 455:1069-1075, 2008; Pao W, Girard N: New driver mutations in non-small-cell lung cancer. Lancet Oncol 12:175-180, 2011; Pao W, Iafrate A J, Su Z: Genetically informed lung cancer medicine. J Pathol 223:230-240, 2011; Kohler L H, Mireskandari M, Kno\ddot{} sel T, et al: FGFR1 expression and gene copy numbers in human lung cancer. Virchows Arch 461:49-57, 2012; Cancer Genome Atlas Research Network: Comprehensive genomic characterization of squamous cell lung cancers. Nature 489:519-525, 2012; Reungwetwattana T, Weroha S J, Molina J R: Oncogenic pathways, molecularly targeted therapies, and highlighted clinical trials in non-small-cell lung cancer (NSCLC). Clin Lung Cancer 13:252-266, 2012) All alterations that were not classified as recurrent were classified as passenger somatic alterations.

Statistical Analysis.

Linear regression analysis was used to study the correlation between mutation frequencies in matched primary and metastatic tumors, considering only mutations found in at least one of the two paired tumor samples. Fisher's exact test was used to compare the proportion of shared alterations in recurrent versus passenger mutations in the matched tumor samples.

Genomic Analyses.

Both array CGH and Sanger sequencing on PIK3CA (exon 10/21) and AKT1 (exon 4) were planned to be performed. DNA was extracted using DNeasy (Qiagen); concentration was determined by using Qubit® 2.0 Fluorometer (Invitrogen). The exons 10 and 21 of PIK3CA gene (NM_{—}006218.2) and exon 4 of AKT1 gene (NM_{—}005163.2) were sequenced using direct Sanger sequencing approach after PCR amplification as previously validated by each platform to cover efficiently mutational hotspot mutation (p.Glu542Lys, p.Glu545Lys p.His1047Arg, p.His1047leu for PI3KCA and p.Glu17Lys for AKT1). Briefly, sequencing was performed after Polymerase Chain Reaction (PCR) amplification of targeted exons and use of the BigDye® Terminator Cycle Sequencing Kit (ref PMID: 22840369). Sequencing reactions were analyzed on 48-capillary 3730 DNA Analyzer®. Sequences reading and alignment were performed with SeqScape® software (Applied Biosystems, Forster City, Calif.). Gene copy number alterations were quantified on Agilent 4*180K or Affymetrix SNP 6.0. For each sample, 500 ng of DNA were fragmented by a double enzymatic digestion (AluI+RsaI) and controlled using 2100 Bioanalyzer System (Agilent Technologies). For genomic analyses on Agilent platforms, tumour DNA and control DNA (Human Genomic DNA Female G152A and Male G147A) were labelled by random priming with CY5-dCTP and CY3-dCTP respectively. They were then hybridised at 65° C. for 17 h. The chips were scanned on an Agilent G2565BA DNA Microarray Scanner and image analysis was done using Feature Extraction V9.1.3 software (Agilent Technologies). Genomic analysis conducted on Affymetrix SNP6.0 arrays were achieved according Affymetrix protocol using 500 ng of DNA as imput. When low amount of genomic DNA was available, 10-30 ng of genomic DNA was used to perform a pre-amplification step using a phi29 modified protocol (Qiagen, REPLI-g Mini Kit, part number 150023, Courtaboeuf, France). To assume robustness of data, a normal genomic DNA was used in any batch of genomic analysis to validate the use of genomic profile of tumor samples. Genomic data are publicly available at Sage Bionetwork (Synapse ID: syn2286494).

Bioinformatic Analyses.

A targetable genomic alteration was defined either as PIK3CA/AKT1 mutation, or an amplification ((Log₂(ratio)≧0.584 on Affymetrix-SNP6, and Log₂(ratio)≧0.887 on Agilent-4x180K)) of a gene encoding for a protein located in a pathway targeted by a drug. The cut-off was chosen based on a previous pilot study (Arnedos et al., 2012, “Array CGH and PIK3CA/AKT1 mutations to drive patients to specific targeted agents: a clinical experience in 108 patients with metastatic breast cancer,” Eur J Cancer 48: 2293-9). The CGH array profile was discussed during a webconference to identify targetable genomic alterations. In addition to mutations and amplifications, in some cases, a gene gain or deletion could be identified as targetable if the CGH array peak was indicative of alteration. For SNP6 data, Log₂ Ratios were computed against hapmap270 using the Affymetrix Genotyping Console™ software. For Agilent data, Log₂ Ratios were computed as Log₂(sample/reference) intensities, after adjusting cyanine signal biases. For each platform, a common workflow was applied with slight platform-specific adjustments for some parameters in the segmentation step. Log₂ Ratios were first centered on their major-left density peak estimated using an expectation-maximisation algorithm (EM) (Chen, et al., 2008, “A probe-density-based analysis method for array CGH data: simulation, normalization and centralization,” Bioinformatics 16: 1749-56). A density peak was defined as major-left if its maximum density was at least 75% of the major peak, and its mean lower than the mean of the major peak. Finally, segmented profiles were obtained using the CBS algorithm (Venkatraman and Olshen. 2007, “A faster circular binary segmentation algorithm for the analysis of array CGH data,” Bioinformatics 6: 657-63). All the analysis were performed in R software (R Core Team, 20130, “R: A language and environment for statistical computing,” R Foundation for Statistical Computing, Vienna, Austria, www.R-project.org).

TABLE 1
Genes with mutations of interest

| Patient | Cpd 1 | | PIK3 | RICT | TP5 | | | | | AKT1 |
|---|---|---|---|---|---|---|---|---|---|---|
| ID | dur. (weeks) | RECIST* | CA | OR | 3 | IGF1R | PTEN | ER+ | PR+ | mut |
| 301-018 | 9 | PR | X | X | X | X | | X | X | |
| 402-003 | 44 | PR | X | | | | | X | X | |
| 009-006 | 4 | SD | | X | | | | X | X | |
| 008-026 | 34 | SD | X | | | | | X | X | |
| 008-028 | 9 | SD | | | | | | X | X | |
| 301-020 | 8 | SD | | | | | | X | X | |
| 301-019 | 6 | SD | | | | | | X | | |
| 402-002 | 21 | SD | | | | | | X | X | |
| 402-004 | 19 | SD | | | | | | X | X | X |
| 008-038 | 7 | SD | | | | | | X | X | |
| 002-030 | 10 | PD | X | | | | X | X | X | |
| 302-004 | 8 | PD | X | | X | | X | X | X | |
| 008-033 | 5 | PD | | | X | | | X | X | |
| 301-021 | 12 | ND | | | | | | X | X | X |
| 201-009 | 1 | NE | | X | | | | X | X | |
| 009-007 | 2 | NE | | | | | | X | | |
| 402-001 | 2 | NE | | | | | | X | Unknown | |

*Response assessment = best target lesion response. Patient 002-030 had overall RECIST response of PD due to new bone lesion; PR = partial response; SD = stable disease; PD = progressive disease; NE = nonevaluable; ND = not done

As can be seen in Table 1, in patients showing a tumor response (PR or SD) upon treatment with Compound 1, mutations were identified in PIK3CA, RICTOR, TP53, AKT1 and/or IGF1R.

### 6.4 Clinical Study B

A Phase 1/2, Multi-Center, Open-Label, Dose Finding Study to Assess the Safety. Tolerability, Pharmacokinetics and Preliminary Efficacy of the mTOR Kinase Inhibitor Compound 1 Administered Orally to Subjects with Advanced Solid Tumors, Non-Hodgkin Lymphoma, or Multiple Myeloma.

Compound 1 is administered orally to subjects with advanced solid tumors, non-Hodgkin lymphoma (NHL), or multiple myeloma (MM). The study is designed as a Phase 1/2 trial consisting of two parts: dose escalation (Part A) and dose expansion (Part B).

The primary objectives of the study are (a) to determine the safety and tolerability of Compound 1 when administered orally and to define the non-tolerated dose (NTD) and the maximum tolerated dose (MTD) and (b) to determine the preliminary pharmacokinetics (PK) of Compound 1 following both single and multiple oral dosing of Compound 1.

The secondary objectives of the study are: (a) to evaluate the extent of inhibition of phosphorylation of S6RP (Ser235/236 and/or Ser240/244) and/or 4EB-P1 (Thr37/46) for mTORC1 activity and AKT (Ser473) and/or other relevant biomarkers for mTORC2 activity in peripheral blood samples and tumor biopsies following treatment with Compound 1; (b) to provide information on the preliminary efficacy of Compound 1; and (c) to characterize PK of the metabolite of Compound 1 following oral dosing of Compound 1.

Compound 1 is administered orally to subjects with advanced solid tumors, non-Hodgkin lymphoma (NHL), or multiple myeloma (MM). The study is designed as a Phase 1/2 trial consisting of two parts: dose escalation (Part A) and dose expansion (Part B).

In Part A, subjects will receive single and multiple ascending doses of Compound 1 to measure pharmacokinetics (PK) and identify the maximum tolerated dose (MTD). A modified accelerated titration design (Simon et al, J Nat Cancer Inst (1997) Vol. 89, No. 15) will be used to identify initial toxicity. During the accelerated course, initial cohorts of one subject will be given Compound 1 in dose increments of 100% until the first instance of first-course grade 2 or higher toxicity suspected to be drug-related, at which point the accelerated phase will stop, and that particular cohort will be expanded to a total of 6 subjects. Subsequently, a standard escalation schedule, with approximately 50% dose increments and 6-subject cohorts, will be initiated in order to establish the non-tolerated dose (NTD) and MTD. Smaller increments and additional subjects within a dose cohort may also be evaluated. A dose will be considered the NTD when 2 evaluable subjects in a cohort experience drug-related DLT. When the NTD is established, dose escalation will stop. The MTD is defined as the last dose level below the NTD with 0 or 1 out of 6 evaluable subjects experiencing DLT during Cycle 1. An intermediate dose (ie, one between the NTD and the last dose level before the NTD) or additional subjects within any dose cohort may be required to more precisely determine the MTD.

In Part B, subjects may start Compound 1 at the MTD and/or a lower dose level based on safety, PK and PD data from Part A. Approximately 200 subjects will be treated and evaluated for safety and preliminary antitumor activity after every two cycles of therapy. Selected tumor types include non-small cell lung cancer (NSCLC), glioblastoma multiforme (GBM), hepatocellular carcinoma (HCC), gastrointestinal neuroendocrine tumor (NET) of non-pancreatic origin, hormone receptor positive breast cancer (HRPBC), diffuse large B-cell lymphoma (DLBCL), and multiple myeloma (MM). Up to 40 evaluable subjects will be enrolled for each tumor type.

Study Population:

Men and women, 18 years or older, with advanced NHL, MM, or advanced unresectable solid tumors, including subjects who have progressed on (or not been able to tolerate) standard therapy or for whom no standard anticancer therapy exists.

Length of Study:

During the first cycle only in Part A, each subject will be administered a single dose of Compound 1 (Day −1), followed by a 48-hour observation and PK sampling period, followed on Day 1 by daily uninterrupted dosing for 28 days (Cycle 1=30 days). In subsequent Part A cycles, subjects are treated in 28-day cycles with continuous dosing from Day 1 to 28. In Part B, subjects receive continuous dosing for 28 days from the beginning—there is neither an initial observation period nor a 48-hour PK collection.

Therapy may be discontinued if there is evidence of disease progression, but subjects can continue to receive Compound 1 as long as the Investigator considers they are deriving benefit from treatment. Therapy will be discontinued if there is unacceptable toxicity or if the subject decides to withdraw from the study.

Enrollment is expected to occur over approximately 36 months. Completion of active treatment and subject follow-up is expected to take up to an additional 24 months.

Study Treatments:

In Part A (the dose escalation phase), the dose level will start at 7.5 mg once daily. After the first dose is administered in any cohort, subjects are observed for at least 30 days before the next higher, protocol-specified dose cohort can begin. Intra-subject dose escalation is not permitted unless approved by the Safety Review Committee (SRC). The total number of subjects in Part A depends on the number of dose cohorts needed to establish the MTD.

In Part B, subjects may receive Compound 1 at the MTD and/or a lower dose level, based on safety, PK and PD evaluations from Part A. Approximately 200 subjects (preselected tumor types in groups of up to 40) evaluable subjects will be evaluated for safety and preliminary antitumor effects.

Overview of Efficacy Assessments:

Subjects will be evaluated for efficacy after every 2 cycles through cycle 6 and every 3 cycles thereafter. The primary efficacy variable is tumor response. Tumor response will be based on investigator assessment using Response Evaluation Criteria in Solid Tumors (RECIST 1.1), International Workshop Criteria (IWC) for NHL/DLBCL, International Uniform Response Criteria for Multiple Myeloma (IURC), or Responses Assessment for Neuro-Oncology (RANO) Working Group for GBM.

Secondary endpoints include mTOR biomarker inhibition in blood and tumor, histopathologic response and correlations with pharmacogenomic findings. Supplementary efficacy variables (eg, ECOG performance status, PET outcomes) will also be examined.

Overview of Safety Assessments:

The safety variables for this study are adverse events, clinical laboratory variables, 12-lead ECGs (centrally reviewed), LVEF assessments, physical examinations, vital signs, concomitant medications/procedure assessments, and pregnancy status.

In Part A, the decision to either evaluate a higher dose level or declare a MTD will be determined by the SRC each time all clinical and laboratory safety data for a given cohort is available for review. The SRC will also determine the dose, doses, or schedule appropriate for Part B. During Part B, the SRC will continue to review safety data regularly and make recommendations about the study continuation, as appropriate.

Overview of Pharmacokinetic Assessments:

The PK profiles of Compound 1 and metabolites will be determined from serial blood and urine collections during the first treatment cycle. These will be correlated with PD outcomes where possible.

Inclusion Criteria:

For both the dose escalation and dose expansion parts of this protocol: (a) understand and voluntarily sign an informed consent document prior to any study related assessments/procedures are conducted; (b) men and women, 18 years or older, with histologically or cytologically-confirmed, advanced NHL, MM, or advanced unresectable solid tumors including subjects who have progressed on (or not been able to tolerate) standard anticancer therapy or for whom no standard anticancer therapy exists; (c) ECOG PS of 0 or 1 for subjects with solid tumors, and 0-2 for hematologic malignancies; (d) subjects must have the following laboratory values: Absolute Neutrophil Count (ANC)≧1.5×10⁹/L; hemoglobin (Hgb)≧9 g/dl; platelets (plt)≧100×10⁹/L; potassium within normal limits or correctable with supplements; AST/SGOT and ALT/SGPT≦2.5× Upper Limit of Normal (ULN) or ≦5.0×ULN if liver tumor is present; serum bilirubin ≦1.5×ULN or ≦2×ULN if liver tumor is present; serum creatinine ≦1.5×ULN or 24-hour clearance ≧50 mL/min; negative serum or urine pregnancy test within 48 hours before starting study treatment in females of childbearing potential; and (e) able to adhere to the study visit schedule and other protocol requirements.

For the dose expansion part (Part B) of this protocol: (a) retrieval of FFPE archival tumor tissue, either in tumor blocks or sectioned/mounted specimens for gene mutation and/or IHC biomarker assay for all tumors except MM. Only in exceptional circumstances may an exemption waiver be granted by the Sponsor for other tumor types; (b) satisfactory screening biopsy for gene mutation and/or IHC biomarker assay for accessible tumors for all tumors except NSCLC and NET (optional), and GBM; (c) Histologically-confirmed tumors of the following types, all with measurable disease. Type-specific criteria are in addition to, or supersede, above criteria where applicable: (i) Non-small cell lung cancer (NSCLC); (ii) Glioblastoma multiforme (GBM) or gliosarcoma, excluding WHO Grade IV oligoastrocytoma: has received prior treatment including radiation and/or chemotherapy, with radiation completed >12 weeks prior to Day 1; planned salvage surgical tumor resection on Day 15±7 days, anticipated to yield ≧200 mg tumor tissue; no prior or scheduled Gliadel® wafer implant unless area of assessment and planned resection is outside the region previously implanted; no prior interstitial brachytherapy or stereotactic radiosurgery unless area of assessment and planned resection is outside the region previously treated; no enzyme-inducing anti-epileptic drugs (EIAED) such as carbamazepine, phenytoin, phenobarbital, or primidone within 14 days before Day 1; able to undergo repeated magnetic resonance imaging (MRI) scans; and availability of adequate FFPE archival tumor material (for PD biomarkers); (iii) Hepatocellular carcinoma (HCC): Plt count ≧60×10⁹/L if portal hypertension is present; Child-Pugh score of less than 7 (ie, class A liver function or better); at least 4 weeks from last dose of α-interferon and/or ribivirin; at least 4 weeks from prior percutaneous ethanol injection, radiofrequency ablation, transarterial embolization, or cryotherapy with documentation of progressive or recurrent disease; (iv) Gastrointestinal neuroendocrine tumor (NET) of non-pancreatic origin: locally unresectable or metastatic well differentiated, low (grade 1) or intermediate (grade 2), non-pancreatic NET or NET of unknown primary origin; pancreatic, NET, pheochromocytomas, paragangliomas, adenocarcinoid and goblet carcinoid tumors, and poorly differentiated, high grade (eg, small cell or large cell) tumors are excluded; symptomatic endocrine-producing tumors and nonfunctional tumors are both allowed; concurrent therapy with somatostatin analogs SSA is required; evidence of radiologic disease progression ≦12 months prior to Cycle 1, Day 1; no receptor targeted radiolabeled therapy ≦3 months prior to Cycle 1, Day 1, no liver-directed therapy ≦4 weeks prior to Cycle 1, Day 1 unless a site of measurable disease other than the treated lesion is present; screening and on-study tumor biopsies are optional in this cohort. Archival tumor collection should be requested, but is not mandatory in this cohort; (v) Hormone receptor-positive breast cancer (HRPBC): unresectable locally advanced or metastatic carcinoma of the breast; ER positive, and HER2/neu negative (0 or 1+), tumor; measurable disease according to RECIST v1.1; at least one year of aromatase inhibitor therapy in the adjuvant setting, or 6 months of aromatase inhibitor therapy for metastatic disease; bisphosphonates or denusomab are allowed in stable doses; cohort may be expanded to enroll a minimum of 5 subjects each with tumors containing PIK3CA mutations; (vi) Multiple Myeloma (MM): measurable levels of myeloma paraprotein in serum (≧0.5 g/dL) or urine (≧0.2 g excreted in a 24-hour collection sample); Absolute Neutrophil Count (ANC)≧1.0×10⁹/L; Platelets (plt)≧60×10⁹/L in subjects in whom ≦50% of bone marrow mononuclear cells are plasma cells or ≧30×10⁹/L in subjects in whom ≧50% of bone marrow mononuclear cells are plasma cells; (vi) Diffuse large B-cell lymphoma (DLBCL): histologically proven diffuse large B-cell non-Hodgkin's lymphoma; Platelets (plt)≧60×10⁹/L for subjects in whom ≦50% of bone marrow mononuclear cells are lymphoma cells, or ≧30×10⁹/L for subjects in whom ≧50% of bone marrow mononuclear cells are lymphoma cells; at least 4 weeks from last dose of therapeutic glucocorticosteroids. Adrenal replacement doses of glucocorticosteroids (up to the equivalent of 10 mg daily prednisone) are allowed.

Exclusion Criteria:

For both the dose escalation and dose expansion parts of this protocol: (a) symptomatic central nervous system metastases (excluding GBM, per Inclusion Criterion 6c). Subjects with brain metastases that have been previously treated and are stable for 6 weeks are allowed; (b) known acute or chronic pancreatitis; (c) subjects with any peripheral neuropathy ≧NCI CTCAE grade 2; (d) subjects with persistent diarrhea or malabsorption ≧NCI CTCAE grade 2, despite medical management; (e) impaired cardiac function or clinically significant cardiac diseases, including any of the following: LVEF<45% as determined by MUGA scan or ECHO; complete left bundle branch, or bifasicular, block; congenital long QT syndrome; persistent or clinically meaningful ventricular arrhythmias or atrial fibrillation; QTcF>460 msec on screening ECG (mean of triplicate recordings); unstable angina pectoris or myocardial infarction ≦3 months prior to starting Compound 1; other clinically significant heart disease such as congestive heart failure requiring treatment or uncontrolled hypertension (blood pressure ≧160/95 mmHg); (f) subjects with diabetes on active treatment or subjects with either of the following: fasting blood glucose ≧126 mg/dL (7.0 mmol/L), or HbA1c≧6.5%; (g) other concurrent severe and/or uncontrolled concomitant medical conditions (eg, active or uncontrolled infection) that could cause unacceptable safety risks or compromise compliance with the protocol; (h) prior systemic cancer-directed treatments or investigational modalities ≦5 half lives or 4 weeks, whichever is shorter, prior to starting study drug or who have not recovered from side effects of such therapy. Subjects must have recovered from any effects of recent radiotherapy that might confound the safety evaluation of study drug; (i) subjects who have undergone major surgery ≦2 weeks prior to starting study drug or who have not recovered from side effects of such therapy; (j) women who are pregnant or breast feeding. Adults of reproductive potential not employing two forms of birth control: females of childbearing potential must agree to use two adequate forms of contraception methods simultaneously (one must be non-hormonal) from the time of giving informed consent until 28 days after the last dose of Compound 1. Females of child-bearing potential, defined as sexually mature women who have not undergone a hysterectomy or bilateral oophorectomy, or who have not been naturally postmenopausal (ie, who have not menstruated at all) for at least 24 consecutive months; males with partners who are female with child-bearing potential must agree that they or their partners will use at least two effective contraceptive methods (including one barrier method) when engaging in reproductive sexual activity throughout the study, and will avoid conceiving for 28 days after the last dose of Compound 1; (k) subjects with known HIV infection; (l) known chronic hepatitis B or C virus (HBV/HCV) infection, unless comorbidity in subjects with HCC; (1) any significant medical condition, laboratory abnormality, or psychiatric illness that would prevent the subject from participating in the study; (m) any condition including the presence of laboratory abnormalities, which places the subject at unacceptable risk if he/she were to participate in the study; and (n) any condition that confounds the ability to interpret data from the study

For the dose expansion part (Part B) of this protocol: concurrent active second malignancy for which the patient is receiving therapy, excluding non-melanomatous skin cancer or carcinoma in situ of the cervix.

Mutational analysis of the clinical samples was performed as described above in section 6.3, using the Foundation Medicine custom artifact databases from 2011 through 2013 (Table 2) and the Foundation Medicine custom artifact database from 2014 (Table 3).

Table 2 and 3 legend: Response assessment=best RECIST overall response; PR=partial response; SD=stable disease; PD=progressive disease; NE=non-evaluable; ND=not done. *: Confirmed reponse. Genes listed multiple times indicate detection of multiple mutations at different locations within the same gene.

TABLE 2
Variants detected using Foundation Medicine custom artifact databases from 2011 through 2013

| | | | | | Localized- | Localized- | | | | |
|---|---|---|---|---|---|---|---|---|---|---|
| | | | | | variants: | variants: | | | | Localized-variants: |
| | | | Days | Best | known- | likely- | | Copy-number- | Copy-number- | variants-of- |
| | | | on | Overall | somatic- | somatic- | Rearrangements: | variants: | variants: | unknown- |
| Site | Pt# | Tumor | Study | response | variants | variants | rearrangements | amplifications | deletions | significance |
| 008 | 26 | Breast | 245 | SD* | PIK3CA | | | MYC | | ALK, ATM, BRCA2, |
| | | | | | | | | | | CHEK2, ESR1, |
| | | | | | | | | | | FLT4, MDM4, |
| | | | | | | | | | | NKX2-1, PIK3CA, |
| | | | | | | | | | | PRKDC |
| 008 | 28 | Breast | 77 | SD | | | BRCA2 | | | APC, ATR, ESR1, |
| | | | | | | | | | | LRP1B, TSC2 |
| 009 | 6 | Breast | 26 | ND | ESR1 | | | CCND1 | | AR, GPR124, |
| | | | | | | | | | | GPR124, GPR124, |
| | | | | | | | | | | IKBKE, MAP2K4, |
| | | | | | | | | | | NOTCH1, NTRK1, |
| | | | | | | | | | | PKHD1, RICTOR |
| 201 | 9 | Breast | 36 | NE | TP53 | | ARID1A | CCND1, ESR1, | | AURKB, CDH5, |
| | | | | | | | | IKBKE, | | EPHB4, RICTOR |
| | | | | | | | | MCL1, | | |
| | | | | | | | | MDM4, MYC, | | |
| | | | | | | | | RICTOR | | |
| 302 | 4 | Breast | 421 | PD | PIK3CA, | | | CCND1, | | MITF, MSH2, SMO |
| | | | | | PTEN, | | | FGFR1 | | |
| | | | | | TP53 | | | | | |
| 301 | 18 | Breast | 65 | PR | PIK3CA, | | | FGFR1, | | FGFR1, FGFR2, |
| | | | | | PIK3CA, | | | IGF1R, | | FGFR2, IDH1, JAK1, |
| | | | | | TP53 | | | PIK3CA | | MET, NOTCH1, |
| | | | | | | | | | | RICTOR, TET2 |
| 301 | 20 | Breast | 63 | SD | ERBB2, | CDH1 | | | | DNMT3A, DOT1L |
| | | | | | ESR1 | | | | | |
| 301 | 19 | Breast | 56 | SD | | BRCA1 | | MYC | CDKN2A, | EPHA7, PRKDC |
| | | | | | | | | | CDKN2B | |
| 402 | 3 | Breast | 384 | PR* | FGFR1, | PTPRD | | | | ATM, CDKN2A, |
| | | | | | PIK3CA | | | | | ESR1, PIK3CA, |
| 402 | 2 | Breast | 146 | SD* | | | | CCND1, MYC | | BRCA2, CTNNB1, |
| | | | | | | | | | | EPHB1, GNAS, |
| | | | | | | | | | | MET, RARA, |
| | | | | | | | | | | TNFAIP3 |
| 402 | 4 | Breast | 157 | SD | | | | | | |
| 301 | 21 | Breast | 85 | ND | AKT1 | | | CCND1 | | AR, NF1, TNKS |
| 008 | 38 | Breast | 56 | SD | | | | | | |
| 002 | 30 | Breast | 86 | PR | CDKN2A, | PTEN | | | | ARID1A, BRCA2, |
| | | | | | ESR1, | | | | | CEBPA, FLT4 |
| | | | | | MLH1, | | | | | |
| | | | | | PIK3CA, | | | | | |
| | | | | | PTEN | | | | | |
| 009 | 7 | Breast | 23 | NE | | | | MCL1, MYC, | | KDR, LTK, SMAD4 |
| | | | | | | | | MYCL1 | | |
| 008 | 33 | Breast | 42 | PD | TP53 | | | CCND1 | | ATM, EPHA5, |
| | | | | | | | | | | GNAS, GNAS, |
| | | | | | | | | | | PHLPP2 |
| 402 | 1 | Breast | 12 | NE | | ATM, | | | | BAP1, CDH5, |
| | | | | | | ESR1 | | | | DPYD, MYCN, |
| | | | | | | | | | | PIK3R1, PRKDC |
| 301 | 10 | DLBCL | 79 | SD | EZH2 | TSC1 | | | | ALK, ATM, BCL2, |
| | | | | | | | | | | BCL2, BCL2, GNAS, |
| | | | | | | | | | | IGF2R, IRS2, IRS2, |
| | | | | | | | | | | MAP2K1, NTRK3, |
| | | | | | | | | | | TGFBR2 |
| 003 | 2 | DLBCL | 83 | PR* | MAP2K1, | TNFAIP3 | | | | APC, AR, CCND3, |
| | | | | | TP53 | | | | | EPHB1, GPR124, |
| | | | | | | | | | | RAF1, SOX10 |
| 202 | 5 | DLBCL | 24 | ND | KIT, TP53 | ARID1A, | | JAK2 | | ATM, BRCA1, |
| | | | | | | TNFAIP3 | | | | ERBB2, GNAQ, |
| | | | | | | | | | | IDH1, MCL1, |
| | | | | | | | | | | PIK3CG |
| 003 | 3 | DLBCL | 198 | PR* | | | | | | |
| 301 | 16 | DLBCL | 23 | ND | | | | | | BRCA2, EPHA5, |
| | | | | | | | | | | GNAS, HSP90AA1, |
| | | | | | | | | | | INSR, NF1, PKHD1, |
| | | | | | | | | | | SMARCA4 |
| 003 | 5 | DLBCL | 44 | PD | | | | | | |
| 003 | 6 | DLBCL | 35 | ND | | | CDKN2A | | | BRCA2, CEBPA, |
| | | | | (clin. | | | | | | FLT4, IGF1R, |
| | | | | prog.) | | | | | | PLCG1, SOX10 |
| 401 | 3 | DLBCL | 35 | ND | CDKN2A, | | | | | ARID1A, CD79B, |
| | | | | | TP53 | | | | | CEBPA, EPHA5, |
| | | | | | | | | | | EPHA5, HSP90AA1, |
| | | | | | | | | | | IGF1R, MLH1, |
| | | | | | | | | | | NTRK1, SMO, TSC1 |
| 003 | 7 | DLBCL | 78 | PD | | | | | | |
| 008 | 30 | DLBCL | 37 | NE | IDH2 | | | | CDKN2A, | CD79B, IDH1, KIT, |
| | | | | | | | | | CDKN2B | MAP2K2, TSC1 |
| 003 | 9 | DLBCL | 41 | ND | | TNFAIP3 | | JAK2 | | BRCA1, BRCA2, |
| | | | | | | | | | | CCND3, CEBPA, |
| | | | | | | | | | | CHEK2, EPHB4, |
| | | | | | | | | | | EPHB6, FLT3, |
| | | | | | | | | | | IKZF1, PTPRD, RB1, |
| | | | | | | | | | | TNKS, TNKS2 |
| 003 | 10 | DLBCL | 33 | NE | | | | | | |
| 010 | 1 | DLBCL | 14 | NE | BRAF, | | | | | BCL2, LRP1B, |
| | | | | | CDKN2A, | | | | | MDM2, PAK3, |
| | | | | | TP53 | | | | | PKHD1 |
| 003 | 13 | DLBCL | 14 | NE | EZH2 | | | AR | | ATM, EPHB6, |
| | | | | | | | | | | MLH1, NPM1, |
| | | | | | | | | | | PTCH2, TNKS2 |
| 009 | 8 | DLBCL | 27 | NE/clin. | | PAX5 | | JAK2 | | ATR, BCL2L1, |
| | | | | prog. | | | | | | BCL6, CD79B, |
| | | | | | | | | | | CDH1, EPHA7, |
| | | | | | | | | | | EPHA7, JAK1, |
| | | | | | | | | | | LRP1B, MCL1, |
| | | | | | | | | | | PTCH1, TSC1 |
| 010 | 2 | DLBCL | 105 | PR | | | | | | AKT1, AKT1, ATR, |
| | | | | | | | | | | ERBB3, FLT1, |
| | | | | | | | | | | NTRK3, NTRK3 |
| 003 | 14 | DLBCL | 63 | SD | | | | | | |
| 003 | 15 | DLBCL | 10 | NE | | | | | | |
| 002 | 31 | DLBCL | 406 | ND | | | | | | CBL, CDKN2A, |
| | | | | | | | | | | FLT3, HRAS, JAK3 |
| 010 | 3 | DLBCL | 58 | PD | EPHB6 | TP53 | | | | CEBPA, LRP1B |
| 401 | 7 | DLBCL | 74 | SD | FBXW7, | RB1 | | | | ABL2, CHEK1, |
| | | | | | TP53 | | | | | ESR1, FLT1, MLL, |
| | | | | | | | | | | MUTYH, TSC1 |
| 401 | 8 | DLBCL | 56 | PD | APC, | | | | FBXW7 | APC, IGF2R, MLH1, |
| | | | | | DNMT3A, | | | | | MSH6 |
| | | | | | TNKS, | | | | | |
| | | | | | TP53 | | | | | |
| 003 | 17 | DLBCL | 10 | NE | | | | | | |
| 010 | 4 | DLBCL | 122 | SD | | | | | | |
| 202 | 6 | DLBCL | 18 | NE | EZH2 | ARID1A | | CDK6, JAK2 | | MSH2, NOTCH1 |
| 402 | 5 | DLBCL | 57 | PD | | | | | | |
| 003 | 18 | DLBCL | 35 | NE | | | | | | |
| 010 | 5 | DLBCL | 87 | PD | BCL6, | EGFR, | | BRAF | MSH6 | ABL2, ABL2, AKT1, |
| | | | | | NOTCH1, | FGFR3, | | | | ATR, AURKA, |
| | | | | | PTEN, | LRP1B, | | | | AURKA, BRCA1, |
| | | | | | TP53, TP53 | PTPRD | | | | CBL, CD79A, CDH2, |
| | | | | | | | | | | CDH2, CDH5, |
| | | | | | | | | | | DOT1L, EPHA7, |
| | | | | | | | | | | EPHB1, EPHB6, |
| | | | | | | | | | | FGFR1, FGFR1, |
| | | | | | | | | | | FGFR3, FGFR3, |
| | | | | | | | | | | GNAS, GUCY1A2, |
| | | | | | | | | | | GUCY1A2, |
| | | | | | | | | | | HSP90AA1, |
| | | | | | | | | | | HSP90AA1, IGF1R, |
| | | | | | | | | | | INHBA, JAK2, |
| | | | | | | | | | | JAK3, LRP1B, |
| | | | | | | | | | | MDM2, MDM2, |
| | | | | | | | | | | MDM2, MEN1, |
| | | | | | | | | | | MRE11A, MYC, |
| | | | | | | | | | | MYC, MYC, NF1, |
| | | | | | | | | | | NOTCH1, PDGFRA, |
| | | | | | | | | | | PDGFRB, PKHD1, |
| | | | | | | | | | | PKHD1, PLCG1, |
| | | | | | | | | | | PRKDC, PTPN11, |
| | | | | | | | | | | RICTOR, RPTOR, |
| | | | | | | | | | | RPTOR, SMARCA4, |
| | | | | | | | | | | SUFU, TET2, TP53, |
| | | | | | | | | | | TSC1, TSC2 |
| 001 | 20 | GBM | 128 | PD | CDKN2A, | NF1 | FGFR3 | | | ATM, CD79A, |
| | | | | | PIK3CA, | | | | | CEBPA, EPHB4, |
| | | | | | TP53 | | | | | JAK2, MSH6, PAX5, |
| | | | | | | | | | | PKHD1 |
| 001 | 22 | GBM | 136 | PD | BRAF, | | | | | EPHA6, NF1 |
| | | | | | NF1, PTEN | | | | | |
| 007 | 1 | GBM | 62 | PD | TP53 | RB1 | | EGFR | | PIK3CG, PIK3R1, |
| | | | | | | | | | | RAF1, TSC2 |
| 007 | 2 | GBM | 22 | NE | | | | EGFR | CDKN2A, | ATR, CDH5, LRP1B, |
| | | | | | | | | | CDKN2B | PDGFRA |
| 007 | 3 | GBM | 82 | PD | | | EGFR | EGFR | CDKN2A, | AR, BCL2A1, EGFR, |
| | | | | | | | | | CDKN2B | FLT3 |
| 007 | 4 | GBM | 41 | PD | EGFR, | | | EGFR | CDKN2A, | ATM, CEBPA, FLT3, |
| | | | | | EGFR | | | | CDKN2B, | RPTOR |
| | | | | | | | | | PTEN | |
| 007 | 5 | GBM | 56 | PD | FGFR1, | | | | CDKN2A, | BRCA2, CD79B, |
| | | | | | PKHD1 | | | | CDKN2B | CEBPA, CEBPA, |
| | | | | | | | | | | GPR124, MEN1, |
| | | | | | | | | | | NF1, PKHD1 |
| 002 | 28 | GBM | 44 | | RB1, TP53 | | EGFR | EGFR | | ATR, CBL, ESR1, |
| | | | | | | | | | | GNAS, LTK, |
| | | | | | | | | | | PIK3CG |
| 007 | 6 | GBM | 23 | NE | | PIK3R1 | | | BRCA2, | PKHD1 |
| | | | | | | | | | CDKN2A, | |
| | | | | | | | | | CDKN2B | |
| 007 | 7 | GBM | 46 | Non | EGFR | | | EGFR | CDKN2A, | AR, GPR124, |
| | | | | prog. | | | | | CDKN2B | LRP1B, NOTCH1, |
| | | | | | | | | | | PRKDC |
| 007 | 8 | GBM | 70 | PD | | | | | | |
| 001 | 24 | GBM | 21 | NE | | | EGFR | EGFR | CDKN2A, | ARID1A, ATM, |
| | | | | | | | | | CDKN2B, | STK11 |
| | | | | | | | | | PTEN | |
| 007 | 10 | GBM | 56 | PD | EGFR, | BRCA2 | | EGFR | CDKN2A, | APC, MLH1, MLL, |
| | | | | | PTEN | | | | CDKN2B | PLCG1, TSC1 |
| 001 | 18 | HCC | 110 | SD* | | | | | | |
| 001 | 23 | HCC | 21 | NE/ND | | | | | | BAP1, CBL, CBL, |
| | | | | | | | | | | FLT1, FLT4, JAK2, |
| | | | | | | | | | | MLL, PHLPP2, |
| | | | | | | | | | | RICTOR, TSC2 |
| 002 | 22 | HCC | 109 | PD | | | | | | |
| 002 | 23 | HCC | 111 | SD | CDKN2A, | | | | | APC, BRCA2, |
| | | | | | CTNNB1 | | | | | EPHB4, ERBB4, |
| | | | | | | | | | | JAK1, LRP1B, |
| | | | | | | | | | | PRKDC, TSC2 |
| 004 | 2 | HCC | 25 | NE (clin. | TP53 | | ABL2 | | PTEN | ATM, ATM, BRAF, |
| | | | | prog.) | | | | | | CDH1, FLT3, |
| | | | | | | | | | | MTOR, PAX5, |
| | | | | | | | | | | PDGFRA |
| 004 | 11 | HCC | 37 | ND | CTNNB1 | | | | | ATM, JAK3, KRAS, |
| | | | | | | | | | | NF1, PHLPP2, |
| | | | | | | | | | | PTCH1 |
| 004 | 10 | HCC | 15 | NE | | | RB1 | | | APC, CDH20, CDH5, |
| | | | | | | | | | | LRP1B, MRE11A, |
| | | | | | | | | | | RICTOR |
| 004 | 12 | HCC | 14 | ND | CTNNB1 | | | | | CHEK2, DOT1L, |
| | | | | | | | | | | ERBB3, MET, |
| | | | | | | | | | | NOTCH1, NTRK1, |
| | | | | | | | | | | PKHD1 |
| 004 | 16 | HCC | 36 | ND | | | | | | AURKB, MSH6, |
| | | | | | | | | | | NOTCH1, NTRK1, |
| | | | | | | | | | | NTRK1, PKHD1, |
| | | | | | | | | | | SMO, TSC1 |
| 006 | 6 | HCC | 105 | PR | TP53 | ARID1A | | CCNE1 | | ALK, ATM, CEBPA, |
| | | | | | | | | | | ERBB4, FGFR3, |
| | | | | | | | | | | GPR124, GPR124, |
| | | | | | | | | | | MCL1, MLL, MLL, |
| | | | | | | | | | | MSH2, PIK3CG, |
| | | | | | | | | | | PKHD1, STK11 |
| 301 | 1 | HCC | 116 | SD* | CTNNB1 | | | EGFR | | GPR124, MCL1, |
| | | | | | | | | | | MLH1, MYCL1, |
| | | | | | | | | | | PKHD1, PRKDC, |
| | | | | | | | | | | RPTOR |
| 302 | 1 | HCC | 112 | SD | TP53 | | | | | ATM, CDH2, CDH5, |
| | | | | | | | | | | DOT1L, FGFR3, |
| | | | | | | | | | | GPR124, IDH1, |
| | | | | | | | | | | JAK3, KIT, MYCN, |
| | | | | | | | | | | NF2, NTRK2, |
| | | | | | | | | | | RICTOR, TSC1 |
| 004 | 20 | HCC | 77 | SD | CTNNB1 | | | | | CEBPA, GNAS, KIT, |
| | | | | | | | | | | KIT, MLL, NF1, SRC |
| 002 | 27 | HCC | 21 | NE/ND | | | | | | |
| 002 | 29 | HCC | 29 | NE/ND | TP53 | CTNNB1 | | | | DDR2, FANCA, |
| | | | | | | | | | | MAP2K4, RICTOR |
| 008 | 18 | HCC | 120 | SD | | TP53 | | | | EPHA7, FLT1, |
| | | | | | | | | | | LRP1B, LRP1B, |
| | | | | | | | | | | MSH6, NF1, PTCH1, |
| | | | | | | | | | | PTCH1, TSC1 |
| 006 | 11 | HCC | 181 | PR* | | | | MYC | CDKN2A, | ABL2, ARID1A, |
| | | | | | | | | | CDKN2B | ATR, BRCA1, |
| | | | | | | | | | | BRCA2, CEBPA, |
| | | | | | | | | | | ERCC2, GPR124, |
| | | | | | | | | | | IGF1R, IKZF1, |
| | | | | | | | | | | JAK2, MITF, RB1, |
| | | | | | | | | | | RICTOR, TP53 |
| 006 | 12 | HCC | 49 | ND | DNMT3A | NF2 | | | | BRCA2, CARD11, |
| | | | | | | | | | | CDK4, DOT1L, |
| | | | | | | | | | | EPHA5, FGFR1, |
| | | | | | | | | | | FLT4, IRS2, MDM2, |
| | | | | | | | | | | NPM1 |
| 006 | 14 | HCC | 14 | NE/ND | | | | | | AR, JAK1, KDR |
| 006 | 15 | HCC | 100 | SD | | | | | | |
| 009 | 1 | HCC | 28 | NE | | ATM, | | | | ALK, FGFR4, |
| | | | | | | FANCA | | | | PDGFRB |
| 009 | 4 | HCC | 34 | NE/ND | | | | | | |
| 009 | 3 | HCC | 164 | SD* | CTNNB1 | | | | | BCL6, EPHA7, |
| | | | | | | | | | | JAK3, PDGFRB, |
| | | | | | | | | | | TSC2 |
| 009 | 16 | HCC | 324 | SD* | | TP53 | | | | CDK8, FLT4, |
| | | | | | | | | | | GPR124, IGF2R, |
| | | | | | | | | | | KIT, PAX5, PKHD1, |
| | | | | | | | | | | TNKS2 |
| 201 | 8 | HCC | 52 | PD | | | | | | |
| 001 | 28 | HCC | 102 | SD | FGFR1, | BRCA2, | | | | ALK, APC, |
| | | exp. | | | TP53, | IKZF1, | | | | APCDD1, ARID1A, |
| | | | | | TRRAP | NOTCH2 | | | | ASXL1, AURKA, |
| | | | | | | | | | | BCL6, BLM, |
| | | | | | | | | | | BRACA1, BRIP1, |
| | | | | | | | | | | CDC73, CHEK1, |
| | | | | | | | | | | CREBBP, CUL4A, |
| | | | | | | | | | | CUL4B, EGFR, |
| | | | | | | | | | | ERBB4, ERBB4, |
| | | | | | | | | | | ERG, FANCM, |
| | | | | | | | | | | FAT3, FAT3, FAT3, |
| | | | | | | | | | | FGF12, FGF7, |
| | | | | | | | | | | FGFR1, FGFR1, |
| | | | | | | | | | | GNAQ, GNAS, |
| | | | | | | | | | | GNAS, GNAS, |
| | | | | | | | | | | GRIN2A, GRIN2A, |
| | | | | | | | | | | IGF1R, IL7R, |
| | | | | | | | | | | INHBA, JAK1, |
| | | | | | | | | | | KDM5A, KDM5C, |
| | | | | | | | | | | KEAP1, KIT, |
| | | | | | | | | | | KLHL6, LRP1B, |
| | | | | | | | | | | LRP1B, LRP1B, |
| | | | | | | | | | | LRP1B, MAP2K4, |
| | | | | | | | | | | MP3K13, MLL2, |
| | | | | | | | | | | MTOR, MTOR, |
| | | | | | | | | | | MYST3, NOTCH1, |
| | | | | | | | | | | NOTCH1, NOTCH1, |
| | | | | | | | | | | NOTCH3, NOTCH3, |
| | | | | | | | | | | NOTCH4, NOTCH4, |
| | | | | | | | | | | NOTCH4, NOTCH4, |
| | | | | | | | | | | NTRK2, NTRK2, |
| | | | | | | | | | | NTRK2, NTRK2, |
| | | | | | | | | | | PAK7, PAK7, PAK7, |
| | | | | | | | | | | PDGFRB, PIK3CG, |
| | | | | | | | | | | PIK3CG, PIK3CG, |
| | | | | | | | | | | PIK3R2, PNRC1, |
| | | | | | | | | | | PRKDC, PTCH1, |
| | | | | | | | | | | RAD51C, RPA1, |
| | | | | | | | | | | RPTOR, RUNX1T1, |
| | | | | | | | | | | RUNX1T1, SH2B3, |
| | | | | | | | | | | SMO, SMO, SYK, |
| | | | | | | | | | | TGFBR2, TIPARP, |
| | | | | | | | | | | TOP1, TRRAP, |
| | | | | | | | | | | TSHR |
| 009 | 11 | HCC | 56 | SD | FANCM | BACH1, | | MYC | | BLM, CDK4, |
| | | exp. | | | | BRCA1, | | | | GATA2, MTOR, |
| | | | | | | BRCA1 | | | | PARP1, SF3B1 |
| 002 | 32 | HCC | 25 | SD | TP53 | | | MYC | | ATR, BRCA2, |
| | | exp. | | | | | | | | CTNNB1, DAXX, |
| | | | | | | | | | | GPR124, MLL, |
| | | | | | | | | | | PDGFRA, RPTOR |
| 009 | 12 | HCC | 8 | NE | | | | | | |
| | | exp. | | | | | | | | |
| 302 | 6 | HCC | 82 | PD | TP53 | | | | TSC2 | CREBBP, ERBB4, |
| | | exp. | | | | | | | | FAT3, KIT, RB1 |
| 301 | 23 | HCC | 70 | SD | TP53 | | | MCL1 | | ATM, NF1, PTCH1, |
| | | exp. | | | | | | | | SOX10, TET2 |
| 001 | 31 | HCC | 117 | SD* | TP53 | | | | | ABL1, ERBB2, |
| | | exp. | | | | | | | | FAT3, MAP3K1, |
| | | | | | | | | | | MET, MET, MET, |
| | | | | | | | | | | PRDM1, PRKDC |
| 006 | 17 | HCC | on- | SD* | | | | MYC, SRC, | | ATM, MLL2, |
| | | exp. | going | | | | | ZNF217 | | NOTCH1, NOTCH3 |
| 006 | 18 | HCC | on- | SD* | CTNNB1, | FAM123B | | CCNE1 | | BRAF, BRCA2, |
| | | exp. | going | | TP53 | | | | | C11orf30, CDK12, |
| | | | | | | | | | | CEBPA, FAM123B, |
| | | | | | | | | | | FANCA, FAT3, |
| | | | | | | | | | | FAT3, FLT3, MLL2, |
| | | | | | | | | | | MSH2, MSH6, |
| | | | | | | | | | | SH2B3, TET2, |
| | | | | | | | | | | UGT1A7 |
| 001 | 33 | HCC | 105 | SD | FGFR2 | | | MCL1 | | CDKN2A, MAP3K1, |
| | | exp. | | | | | | | | TOP1 |
| 001 | 34 | HCC | 294 | SD* | MTOR, | | | MYC, MYST3 | | BRCA1, DNMT3A, |
| | | exp. | | | TSC2 | | | | | GNAS, MLL, |
| | | | | | | | | | | MYCL1, MYCL1, |
| | | | | | | | | | | NFE2L2, PRKDC, |
| | | | | | | | | | | PTEN, TRRAP |
| 001 | 32 | HCC | 48 | ND | TP53 | | ATM | CCND1, | | ALK, CDKN2C, |
| | | exp. | | | | | | FGF19, FGF3, | | CTNNB1 |
| | | | | | | | | FGF4 | | |
| 009 | 14 | HCC | 54 | PD | | | | | | |
| | | exp. | | | | | | | | |
| 008 | 45 | HCC | 136 | SD | | | | | | |
| | | exp. | | | | | | | | |
| 009 | 19 | HCC | 53 | PD | TP53 | ARID2, | | | | CEBPA, CHUK, |
| | | exp. | | | | ARID2, | | | | KDM5A, LRP1B, |
| | | | | | | CSF1R | | | | MPL, PARP1, |
| | | | | | | | | | | SH2B3, TET2, |
| | | | | | | | | | | ZNF703 |
| 002 | 37 | HCC | on- | SD* | MSH6, | ATRX, | | | | ARAF, ATR, ATR, |
| | | exp. | going | | TP53 | FLT3, | | | | AXL, CARD11, |
| | | | | | | NOTCH3, | | | | CDK6, CIC, EPHB1, |
| | | | | | | RB1 | | | | FANCM, FAT3, |
| | | | | | | | | | | FLT1, FLT4, IKZF1, |
| | | | | | | | | | | KDR, MAP2K1, |
| | | | | | | | | | | MAP2K2, MEF2B, |
| | | | | | | | | | | MLL2, NF1, NF1, |
| | | | | | | | | | | NKX2-1, NOTCH1, |
| | | | | | | | | | | NSD1, NTRK2, |
| | | | | | | | | | | PAK7, PAK7, |
| | | | | | | | | | | PARP3, PARP4, |
| | | | | | | | | | | PDGFRA, PDGFRA, |
| | | | | | | | | | | PRKDC, PRKDC, |
| | | | | | | | | | | RB1, RUNX1T1, |
| | | | | | | | | | | RUNX1T1, |
| | | | | | | | | | | SMARCA4, SPEN, |
| | | | | | | | | | | SPOP, TSC1 |
| 301 | 24 | HCC | 112 | SD | ATM, | | | | | KDM5C, PARP2, |
| | | exp. | | | CTNNB1 | | | | | PARP4, PARP4, |
| | | | | | | | | | | PARP4, PRKDC, |
| | | | | | | | | | | RAD50 |
| 001 | 35 | HCC | 114 | SD | PIK3CA | | | | | BRCA1, CCND1, |
| | | exp. | | | | | | | | MLL, NOTCH3, |
| | | | | | | | | | | PTCH1, PTCH1 |
| 401 | 10 | HCC | on- | SD* | | | | | | |
| | | exp. | going | | | | | | | |
| 003 | 20 | HCC | 45 | ND | | | | | | |
| | | exp. | | | | | | | | |
| 006 | 19 | HCC | 144 | SD | | | | | | |
| | | | or | | | | | | | |
| | | exp. | 222 | | | | | | | |
| 401 | 11 | HCC | 133 | SD | | | | | | AR, FAT3, INHBA, |
| | | exp. | | | | | | | | NOTCH2, RPTOR, |
| | | | | | | | | | | ZNF703 |
| 002 | 36 | HCC | on- | SD | ERBB4, | | FANCA | KDR, KIT, | | BRCA1, KDM5A, |
| | | exp. | going | | TP53, TP53 | | | PDGFRA, | | KDM5A, MDH6, |
| | | | | | | | | RICTOR | | NOTCH2 |
| 002 | 38 | HCC | on- | NE | | | | | | |
| | | exp. | going | | | | | | | |
| 001 | 36 | HCC | 47 | ND | CTNNB1 | | | MYC | | FANCE, FAT3, |
| | | exp. | | | | | | | | MAP3K1, MAP3K1, |
| | | | | | | | | | | NTRK2, NUP93, |
| | | | | | | | | | | NUP93 |
| 006 | 20 | HCC | on- | PR* | AKT1, | ASXL1 | | | CDKN2A | ATRX, BRCA1, |
| | | exp. | going | | CTNNB1, | | | | | CREBBP, CTNNA1, |
| | | | | | KRAS | | | | | GPR124, IGF1R, |
| | | | | | | | | | | LRP1B, MAP3K13, |
| | | | | | | | | | | MITF, MSH2, |
| | | | | | | | | | | NTRK2, SETD2, |
| | | | | | | | | | | TNFAIP3, ZNF217 |
| 002 | 39 | HCC | 28 | NE | TP53 | ATM | | CCND1, | | APC, ATM, |
| | | exp. | | | | | | FGF19, FGF3, | | CARD11, GRIN2A, |
| | | | | | | | | FGF4 | | LRP1B, PARP4 |
| 402 | 6 | HCC | 36 | NE | DNMT3A, | PTEN | | CCND1, | | AKT2, ERBB3, |
| | | exp. | | | MSH6, | | | FGF19, FGF3, | | FAT3, FGFR4, |
| | | | | | PTEN, | | | FGF4 | | FIP1L1, GNAS, |
| | | | | | TP53 | | | | | IGF2, IRS2, NF1, |
| | | | | | | | | | | NTRK2, PRKDC, |
| | | | | | | | | | | RAD51L3, TRRAP |
| 002 | 25 | MM | 21 | NE | | | | | | |
| 003 | 1 | MM | 35 | PD | | | | | | |
| 201 | 3 | MM | 76 | PD | TP53 | | | | | FGFR3, FGFR3, |
| | | | | | | | | | | PTPRD, PTPRD, |
| | | | | | | | | | | PTPRD |
| 202 | 1 | MM | 66 | SD | KRAS | | | | | ATM, AURKB, |
| | | | | | | | | | | BCL6, CDH20, |
| | | | | | | | | | | LRP1B, LRP1B, |
| | | | | | | | | | | MET, TET2 |
| 301 | 4 | MM | 90 | SD | | | | | | |
| 301 | 3 | MM | 127 | SD | KRAS | | | | | ATM, ATR, CHEK2, |
| | | | | | | | | | | DDR2, EPHB1, |
| | | | | | | | | | | IDH1, IKBKE, KIT |
| 301 | 8 | MM | 348 | SD | | | | | | CDH20, CDH20, |
| | | | | | | | | | | CDH5, CDH5, IDH1, |
| | | | | | | | | | | IDH1, PKHD1, |
| | | | | | | | | | | PKHD1, PRKDC, |
| | | | | | | | | | | PRKDC |
| 301 | 9 | MM | 43 | PD | BRAF | | | | | CDKN2A, INHBA, |
| | | | | | | | | | | LTK, MET, PKHD1, |
| | | | | | | | | | | RET, RET, |
| | | | | | | | | | | SMARCA4, TSC1 |
| 008 | 10 | MM | 12 | NE | | | | | | |
| 301 | 12 | MM | 46 | SD | BRAF | | | | | CBL, CDKN2C, |
| | | | | | | | | | | HSP90AA1, LRP1B, |
| | | | | | | | | | | SMARCA4 |
| 301 | 13 | MM | 48 | PD | | | | | | |
| 008 | 14 | MM | 28 | NE | | | | | | |
| 202 | 3 | MM | 42 | PD | KRAS | DNMT3A | | | | MCL1, NOTCH1 |
| 202 | 4 | MM | 7 | NE | PTPN11 | | | BRAF | RB1 | ATM, EPHB4, LRP6, |
| | | | | | | | | | | MYCN |
| 008 | 16 | NET | on- | PR* | | | | | | |
| | | | going | | | | | | | |
| 008 | 21 | NET | 174 | PR* | | | | | | |
| 008 | 22 | NET | 174 | SD* | | | | | | |
| 008 | 27 | NET | 444 | SD* | | | | | | |
| 008 | 24 | NET | 262 | SD* | | | | | | ALK, CEBPA, IDH1 |
| 008 | 23 | NET | 21 | NE | | | | | | |
| 008 | 25 | NET | on- | SD* | | | | | | ARID1A, ARID1A, |
| | | | going | | | | | | | ATM, ESR1, JAK3, |
| | | | | | | | | | | NTRK1, PRKDC, |
| | | | | | | | | | | SMO |
| 001 | 25 | NET | 127 | SD* | | | | | | |
| 401 | 2 | NET | on- | SD* | | | | | | JUN, MSH6, SMO |
| | | | going | | | | | | | |
| 001 | 26 | NET | 319 | SD* | | | | | | |
| 008 | 32 | NET | on- | SD* | | | | | | |
| | | | going | | | | | | | |
| 008 | 31 | NET | on- | SD* | | | | | | |
| | | | going | | | | | | | |
| 004 | 24 | NET | 17 | NET | EPHA7, | MEN1, | | AURKB | | FLT4, GNAS, TSC2 |
| | | | | | PTEN | SMARCA | | | | |
| | | | | | | 4 | | | | |
| 004 | 25 | NET | on- | SD* | | | | | | |
| | | | going | | | | | | | |
| 008 | 35 | NET | 252 | SD* | | | | | | |
| 008 | 34 | NET | 420 | SD | | | | | | |
| 401 | 4 | NET | 7 | NE | | | | | | |
| 401 | 5 | NET | 423 | SD | | | | | | |
| 401 | 6 | NET | 115 | SD* | CDKN2A, | | | | | AR, GPR124 |
| | | | | | FGFR1 | | | | | |
| 008 | 36 | NET | on- | SD* | | | | | | ALK, AR, ARID1A, |
| | | | going | | | | | | | FOXP4, GNAS, |
| | | | | | | | | | | KDM6A, KDM6A |
| 009 | 9 | NET | 50 | NE | | | | | | |
| 008 | 17 | NET | on- | SD* | | | | | | |
| | | | going | | | | | | | |
| 008 | 37 | NET | on- | SD* | | | | | | |
| | | | going | | | | | | | |
| 001 | 29 | NET | 77 | SD | | | | | | |
| | | exp. | | | | | | | | |
| 009 | 10 | NET | 86 | NE | | | | | | |
| | | exp. | | | | | | | | |
| 002 | 33 | NET | 119 | SD | | | | | | |
| | | exp. | | | | | | | | |
| 008 | 40 | NET | 200 | SD* | | | | | | |
| | | exp. | | | | | | | | |
| 009 | 13 | NET | on- | SD* | | | | | | |
| | | exp. | going | | | | | | | |
| 201 | 10 | NET | 56 | PD | | | | | | |
| | | exp. | | | | | | | | |
| 008 | 42 | NET | 51 | SD | | | | | | |
| | | exp. | | | | | | | | |
| 008 | 44 | NET | 275 | SD | | | | | | |
| | | exp. | | | | | | | | |
| 008 | 39 | NET | 245 | SD* | | | | | | |
| | | exp. | | | | | | | | |
| 401 | 9 | NET | 42 | SD | | | | | | |
| | | exp. | | | | | | | | |
| 003 | 19 | NET | 55 | SD | | | | | | |
| | | exp. | | | | | | | | |
| 009 | 15 | NET | 15 | ND | | | | | | |
| | | exp. | | | | | | | | |
| 008 | 49 | NET | on- | SD* | | | | | | |
| | | exp. | going | | | | | | | |
| 008 | 52 | NET | on- | SD | | | | | | |
| | | exp. | going | | | | | | | |
| 008 | 43 | NET | on- | SD* | | | | | | |
| | | exp. | going | | | | | | | |
| 008 | 48 | NET | 245 | SD* | | | | | | |
| | | exp. | | | | | | | | |
| 008 | 47 | NET | 33 | ND | | | | | | |
| | | exp. | | | | | | | | |
| 008 | 51 | NET | on- | SD* | | | | | | |
| | | exp. | going | | | | | | | |
| 008 | 50 | NET | 368 | SD* | | | | | | |
| | | exp. | | | | | | | | |
| 008 | 46 | NET | on- | SD* | | | | | | |
| | | exp. | going | | | | | | | |
| 002 | 35 | NET | 106 | SD | | | | | | |
| | | exp. | | | | | | | | |
| 009 | 18 | NET | 180 | SD | | | | | | |
| | | exp. | | | | | | | | |
| 008 | 53 | NET | on- | SD* | | | | | | |
| | | exp. | going | | | | | | | |
| 301 | 7 | NSCL+ | 299 | PR* | TP53 | STK11 | | EGFR | | AKT1, AR, ARID1A, |
| | | | | | | | | | | BRCA2, CARD11, |
| | | | | | | | | | | CDKN2A, CDKN2A, |
| | | | | | | | | | | DDR2, EPHA6, |
| | | | | | | | | | | FBXW7, FBXW7, |
| | | | | | | | | | | FGFR2, FLT4, |
| | | | | | | | | | | INHBA, JAK3, KIT, |
| | | | | | | | | | | LRP1B, LRP6, |
| | | | | | | | | | | NTRK1, PDGFRB, |
| | | | | | | | | | | RICTOR, |
| | | | | | | | | | | SMARCA4, SUFU, |
| | | | | | | | | | | TBX22, TNFAIP3, |
| | | | | | | | | | | TOP1, TSC2 |
| 302 | 2 | NSCL+ | 55 | ND | KRAS, | ATM | | NKX2-1 | | AR, ATM, ATM, |
| | | | | | STK11, | | | | | HSP90AA1, IGF2R, |
| | | | | | TP53 | | | | | IKBKE, KIT, LRP1B, |
| | | | | | | | | | | LRP1B, LRP1B, |
| | | | | | | | | | | RUNX1, STK11 |
| 002 | 90 | NSCLC | 24 | ND | EGFR, | | | EGFR, MYC | CDKN2A, | AKT2, ERBB3, |
| | | | | (clinical | EGFR | | | | CDKN2B | GPR124, IDH1, |
| | | | | prog.) | | | | | | INSR, LTK, TP53 |
| 002 | 21 | NSCLC | 164 | SD* | | TNFAIP3 | | BCL2L1, | | CARD11, CDH20, |
| | | | | | | | | CCND1 | | FGFR1, GPR124, |
| | | | | | | | | | | IGF2R, KIT, KIT, |
| | | | | | | | | | | LRP1B, MLL, NF1, |
| | | | | | | | | | | RARA, RET |
| 004 | 5 | NSCLC | 155 | SD* | | | | | | |
| 004 | 6 | NSCLC | 56 | SD | KRAS | | | | | ATM, FLT1, LRP1B, |
| | | | | | | | | | | NF1, RARA, TOP1 |
| 004 | 8 | NSCLC | 28 | NE | TP53 | | | | | CEBPA, EPHA7, |
| | | | | | | | | | | GNAS, LRP1B, |
| | | | | | | | | | | MAP2K2, PKHD1, |
| | | | | | | | | | | PRKDC |
| 004 | 14 | NSCLC | 60 | PD | CDKN2A | | | | | ATR, DOT1L, |
| | | | | | | | | | | EPHA6, EPHB1, |
| | | | | | | | | | | EPHB1, ERBB3, |
| | | | | | | | | | | JAK3, MYC, |
| | | | | | | | | | | NOTCH1, NTRK1, |
| | | | | | | | | | | PRKDC, PRKDC, |
| | | | | | | | | | | RET |
| 006 | 3 | NSCLC | 83 | SD | TP53 | APC | BRCA1 | NKX2-1 | | ATM, EPHB6, |
| | | | | | | | | | | GPR124, GUCY1A2, |
| | | | | | | | | | | LRP1B, PIK3CG, |
| | | | | | | | | | | PLCG1, SMO, |
| | | | | | | | | | | TNFAIP3 |
| 006 | 2 | NSCLC | 26 | NE | TP53 | | | ERBB2 | SMAD4 | ATM, ATM, ERBB4, |
| | | | | | | | | | | FANCA, KIT, MLL, |
| | | | | | | | | | | MLL, MUTYH, |
| | | | | | | | | | | MYC, NKX2-1, |
| | | | | | | | | | | TSC2 |
| 006 | 5 | NSCLC | 28 | NE | MAP2K1 | | | | | CEBPA, FANCA, |
| | | | | | | | | | | FLT4, GUCY1A2, |
| | | | | | | | | | | KIT, LTK, MAP2K2, |
| | | | | | | | | | | NTRK1, PKHD1, |
| | | | | | | | | | | PKHD1, TET2 |
| 006 | 8 | NSCLC | 22 | NE | EGFR | | | MDM2 | | APC, ATM, ATM, |
| | | | | | | | | | | FLT4, IRS2, KIT, |
| | | | | | | | | | | PKHD1, TNFAIP3 |
| 006 | 7 | NSCLC | 126 | SD | ERBB2 | | | | | ATM, FLT4, MEN1, |
| | | | | | | | | | | MLL, NKX2-1, |
| | | | | | | | | | | NKX2-1, PIK3R1, |
| | | | | | | | | | | TSC2, USP9X |
| 006 | 4 | NSCLC | 70 | ND | VHL | | | | | BAP1, CEBPA, |
| | | | | | | | | | | KDR, PRKDC, |
| | | | | | | | | | | PTCH1, TGFBR2 |
| 006 | 9 | NSCLC | 120 | SD | TP53 | DNMT3A | | | | AKT3, ALK, ALK, |
| | | | | | | | | | | AR, CBL, CRKL, |
| | | | | | | | | | | EPHA3, EPHA5, |
| | | | | | | | | | | EPHB1, EPHB6, |
| | | | | | | | | | | EPHB6, FLT1, |
| | | | | | | | | | | GNAS, LRP1B, |
| | | | | | | | | | | LRP1B, MET, |
| | | | | | | | | | | MTOR, NF1, |
| | | | | | | | | | | NTRK1, NTRK2, |
| | | | | | | | | | | PDGFRB, SMO, |
| | | | | | | | | | | TET2, TSC2 |
| 006 | 10 | NSCLC | 28 | NE | KRAS, | | | KRAS, MCL1, | | APC, ATM, BCL2L2, |
| | | | | | TP53 | | | SRC | | BRCA1, CD79B, |
| | | | | | | | | | | CDK6, EPHA7, |
| | | | | | | | | | | GNAS, KIT, LRP1B, |
| | | | | | | | | | | MITF, NKX2-1, |
| | | | | | | | | | | NOTCH1, NTRK1, |
| | | | | | | | | | | PDGFRA, SMAD4 |
| 201 | 4 | NSCLC | 69 | SD | TP53 | PTEN | | FGFR1, | | ARID1A, ATM, |
| | | | | | | | | PIK3CA, | | FGFR1, FLT4, KIT, |
| | | | | | | | | SOX2 | | LRP1B, LRP1B, |
| | | | | | | | | | | MSH2, NOTCH1, |
| | | | | | | | | | | NTRK3, PHLPP2, |
| | | | | | | | | | | PRKDC, RAF1, |
| | | | | | | | | | | SMARCA4 |
| 201 | 5 | NSCLC | 229 | SD* | TP53 | SMARCA | | | | ALK, ARID1A, ATR, |
| | | | | | | 4 | | | | CEBPA, FLT1, |
| | | | | | | | | | | GNAS, GUCY1A2, |
| | | | | | | | | | | IDH1, KIT, MLL, |
| | | | | | | | | | | MSH2, NF2, NTRK1, |
| | | | | | | | | | | PIK3CG, PKHD1, |
| | | | | | | | | | | PTPRD, SMARCA4 |
| 301 | 5 | NSCLC | 170 | SD | | TP53 | | | | EPHA5, EPHB6, |
| | | | | | | | | | | ERG, KIT, NF1 |
| 201 | 6 | NSCLC | 8 | NE | PIK3CA, | | | MET | | ATM, ATM, ATM, |
| | | | | | PIK3CA, | | | | | ATM, EPHA5, |
| | | | | | PTEN, | | | | | EPHA5, FANCA, |
| | | | | | TP53, TP53 | | | | | FANCA, PIK3CG, |
| | | | | | | | | | | PIK3CG, PIK3R1, |
| | | | | | | | | | | PIK3R1, PKHD1, |
| | | | | | | | | | | PKHD1, PTCH2, |
| | | | | | | | | | | PTCH2, USP9X, |
| | | | | | | | | | | USP9X |
| 004 | 17 | NSCLC | 35 | NE | | | | MCL1 | | APC, CARD11, |
| | | | | | | | | | | CARD11, FANCA, |
| | | | | | | | | | | HSP90AA1, KDM6A |
| 008 | 3 | NSCLC | 76 | NE | BRAF, | | | | | APC, BRCA2, |
| | | | | | CDKN2A, | | | | | IGF2R, MLL, MSH2, |
| | | | | | TP53 | | | | | MSH2, SMO |
| 008 | 2 | NSCLC | 28 | NE | | | ERG | | TP53 | ATM, AURKB, |
| | | | | | | | | | | IDH1, MLL, PRKDC |
| 201 | 7 | NSCLC | 19 | ND | ARID1A | ARID1A | | MCL1, | | ABL2, ATM, |
| | | | | | | | | PIK3CA, | | EPHA5, EPHB6, |
| | | | | | | | | RICTOR, | | FGFR1, FGFR2, |
| | | | | | | | | SOX2 | | GNAS, IGF2R, |
| | | | | | | | | | | LRP1B, MET, MLL, |
| | | | | | | | | | | MLL, MLL, MTOR, |
| | | | | | | | | | | PIK3CG, PKHD1, |
| | | | | | | | | | | PRKDC, TGFBR2 |
| 301 | 6 | NSCLC | 58 | SD | | | | | | |
| 008 | 12 | NSCLC | 140 | SD* | TP53 | PTEN, | | | | FLT4, HSP90AA1, |
| | | | | | | RB1 | | | | JAK2, KIT, PAK3, |
| | | | | | | | | | | PKHD1, RB1, |
| | | | | | | | | | | STAT3 |

TABLE 3
Variants detected using Foundation Medicine custom artifact database from 2014

| | | | | | Localized- | Localized- | | | | Localized- |
|---|---|---|---|---|---|---|---|---|---|---|
| | | | | | variants: | variants: | | | | variants: |
| | | | Days | Best | known- | likely- | | Copy-number- | Copy-number- | variants-of- |
| | | | on | overall | somatic- | somatic- | Rearrangements: | variants: | variants: | unknown- |
| Site | Pt# | Tumor | Study | response | variants | variants | rearrangements | amplifications | deletions | significance |
| 002 | 030 | Breast | 86 | PD | CDKN2A, | PTEN | | | | ARID1A, BRCA2, |
| | | | | | ESR1, | | | | | CEBPA, FLT4 |
| | | | | | MLH1, | | | | | |
| | | | | | PIK3CA, | | | | | |
| | | | | | PTEN | | | | | |
| 008 | 026 | Breast | 245 | SD | PIK3CA | | | MYC | | ALK, BRCA2, |
| | | | | | | | | | | CHEK2, ESR1, |
| | | | | | | | | | | FLT4, MDM4, |
| | | | | | | | | | | NKX2-1, PIK3CA, |
| | | | | | | | | | | PRKDC |
| 008 | 028 | Breast | 77 | SD | | | BRCA2 | | | ATR, ESR1, LRP1B, |
| | | | | | | | | | | TSC2 |
| 008 | 033 | Breast | 42 | PD | TP53 | | | CCND1 | | ATM, EPHA5, |
| | | | | | | | | | | GNAS, GNAS, |
| | | | | | | | | | | PHLPP2 |
| 008 | 038 | Breast | 56 | SD | | | | | | |
| 009 | 006 | Breast | 26 | SD | ESR1 | | | CCND1 | | AR, GPR124, |
| | | | | | | | | | | GPR124, GPR124, |
| | | | | | | | | | | IKBKE, MAP2K4, |
| | | | | | | | | | | NTRK1, PKHD1, |
| | | | | | | | | | | RICTOR |
| 009 | 007 | Breast | 23 | PD | | | | MCL1, MYC, | | KDR, LTK, SMAD4 |
| | | | | | | | | MYCL1 | | |
| 201 | 009 | Breast | 36 | NE | TP53 | | ARID1A | CCND1, ESR1, | | AURKB, CDH5, |
| | | | | | | | | IKBKE, | | EPHB4, RICTOR |
| | | | | | | | | MCL1, | | |
| | | | | | | | | MDM4, MYC, | | |
| | | | | | | | | RICTOR | | |
| 301 | 018 | Breast | 65 | PR | PIK3CA, | | | FGFR1, | | FGFR1, FGFR2, |
| | | | | | PIK3CA, | | | IGF1R, | | FGFR2, IDH1, JAK1, |
| | | | | | TP53 | | | PIK3CA | | MET, NOTCH1, |
| | | | | | | | | | | RICTOR, TET2 |
| 301 | 019 | Breast | 56 | SD | | BRCA1 | | MYC | CDKN2A, | EPHA7, PRKDC |
| | | | | | | | | | CDKN2B | |
| 301 | 020 | Breast | 63 | SD | ERBB2, | CDH1 | | | | DNMT3A, DOT1L |
| | | | | | ESR1 | | | | | |
| 301 | 021 | Breast | 85 | SD | AKT1 | | | CCND1 | | AR, NF1, TNKS |
| 302 | 004 | Breast | 56 | PD | PIK3CA, | | | CCND1, | | MITF, SMO |
| | | | | | PTEN, | | | FGFR1 | | |
| | | | | | TP53 | | | | | |
| 402 | 001 | Breast | 12 | NE | | ATM, | | | | BAP1, CDH5, |
| | | | | | | ESR1 | | | | DPYD, MYCN, |
| | | | | | | | | | | PIK3R1, PRKDC |
| 402 | 002 | Breast | 146 | SD | | | | CCND1, MYC | | BRCA2, CTNNB1, |
| | | | | | | | | | | EPHB1, GNAS, |
| | | | | | | | | | | MET, RARA, |
| | | | | | | | | | | TNFAIP3 |
| 402 | 003 | Breast | 384 | PR | FGFR1, | PTPRD | | | | ATM, CDKN2A, |
| | | | | | PIK3CA | | | | | ESR1, PIK3CA |
| 402 | 004 | Breast | 157 | SD | KRAS | | | | | AR, ATM, CDH20, |
| | | | | | | | | | | PKHD1, TGFBR2, |
| | | | | | | | | | | VHL |
| 002 | 031 | DLBCL | 406 | NE | | | | | | CBL, CDKN2A, |
| | | | | | | | | | | FLT3, HRAS, JAK3 |
| 003 | 002 | DLBCL | 83 | PR | MAP2K1, | TNFAIP3 | | | | APC, AR, CCND3, |
| | | | | | TP53 | | | | | EPHB1, GPR124, |
| | | | | | | | | | | RAF1, SOX10 |
| 003 | 003 | DLBCL | 198 | PR | | | | | CDKN2A, | TSC1 |
| | | | | | | | | | CDKN2B | |
| 003 | 005 | DLBCL | 44 | PD | | | | | | |
| 003 | 006 | DLBCL | 35 | ND | | | CDKN2A | | | BRCA2, CEBPA, |
| | | | | | | | | | | FLT4, IGF1R, |
| | | | | | | | | | | PLCG1, SOX10 |
| 003 | 007 | DLBCL | 78 | PR | | | | | | |
| 003 | 009 | DLBCL | 41 | PD | | TNFAIP3 | | JAK2 | | BRCA1, BRCA2, |
| | | | | | | | | | | CCND3, CEBPA, |
| | | | | | | | | | | CHEK2, EPHB4, |
| | | | | | | | | | | EPHB6, IKZF1, |
| | | | | | | | | | | PTPRD, RB1, TNKS, |
| | | | | | | | | | | TNKS2 |
| 003 | 010 | DLBCL | 33 | NE | | | | | | |
| 003 | 013 | DLBCL | 14 | PD | EZH2 | | | AR | | ATM, EPHB6, |
| | | | | | | | | | | MLH1, NPM1, |
| | | | | | | | | | | PTCH2, TNKS2 |
| 003 | 014 | DLBCL | 63 | SD | | | | | | |
| 003 | 015 | DLBCL | 10 | PD | | | | | | |
| 003 | 017 | DLBCL | 10 | PD | | | | | | |
| 003 | 018 | DLBCL | 35 | ND | | | | | | |
| 008 | 030 | DLBCL | 37 | ND | IDH2 | | | | CDKN2A, | CD79B, MAP2K2, |
| | | | | | | | | | CDKN2B | TSC1 |
| 009 | 008 | DLBCL | 27 | PD | | PAX5 | | JAK2 | | ATR, BCL2L1, |
| | | | | | | | | | | BCL6, CD79B, |
| | | | | | | | | | | EPHA7, EPHA7, |
| | | | | | | | | | | JAK1, LRP1B, |
| | | | | | | | | | | MCL1, TSC1 |
| 010 | 001 | DLBCL | 14 | PD | BRAF, | | | | | BCL2, LRP1B, |
| | | | | | CDKN2A, | | | | | MDM2, PAK3, |
| | | | | | TP53 | | | | | PKHD1 |
| 010 | 002 | DLBCL | 105 | PR | | | | | | AKT1, AKT1, ATR, |
| | | | | | | | | | | ERBB3, FLT1, |
| | | | | | | | | | | NTRK3, NTRK3 |
| 010 | 003 | DLBCL | 58 | PD | EPHB6 | TP53 | | | | CEBPA, LRP1B |
| 010 | 004 | DLBCL | 122 | SD | | | | | | |
| 010 | 005 | DLBCL | 87 | PD | BCL6, | EGFR, | | BRAF | MSH6 | ABL2, ABL2, AKT1, |
| | | | | | NOTCH1, | FGFR3, | | | | ATR, AURKA, |
| | | | | | PTEN, | LRP1B, | | | | AURKA, BRCA1, |
| | | | | | TP53, TP53 | PTPRD | | | | CBL, CD79A, CDH2, |
| | | | | | | | | | | CDH2, CDH5, |
| | | | | | | | | | | DOT1L, EPHA7, |
| | | | | | | | | | | EPHB1, EPHB6, |
| | | | | | | | | | | FGFR1, FGFR1, |
| | | | | | | | | | | FGFR3, FGFR3, |
| | | | | | | | | | | GNAS, GUCY1A2, |
| | | | | | | | | | | GUCY1A2, |
| | | | | | | | | | | HSP90AA1, |
| | | | | | | | | | | HSP90AA1, IGF1R, |
| | | | | | | | | | | INHBA, JAK2, |
| | | | | | | | | | | JAK3, LRP1B, |
| | | | | | | | | | | MDM2, MDM2, |
| | | | | | | | | | | MDM2, MEN1, |
| | | | | | | | | | | MRE11A, MYC, |
| | | | | | | | | | | MYC, MYC, NF1, |
| | | | | | | | | | | NOTCH1, PDGFRA, |
| | | | | | | | | | | PDGFRB, PKHD1, |
| | | | | | | | | | | PKHD1, PLCG1, |
| | | | | | | | | | | PRKDC, PTPN11, |
| | | | | | | | | | | RICTOR, RPTOR, |
| | | | | | | | | | | RPTOR, SMARCA4, |
| | | | | | | | | | | SUFU, TET2, TP53, |
| | | | | | | | | | | TSC1, TSC2 |
| 202 | 005 | DLBCL | 24 | PD | KIT, TP53 | ARID1A, | | JAK2 | | BRCA1, ERBB2, |
| | | | | | | TNFAIP3 | | | | GNAQ, MCL1, |
| | | | | | | | | | | PIK3CG |
| 202 | 006 | DLBCL | 18 | PD | EZH2 | ARID1A | | CDK6, JAK2 | | MSH2, NOTCH1 |
| 301 | 010 | DLBCL | 79 | SD | EZH2 | TSC1 | | | | ALK, BCL2, BCL2, |
| | | | | | | | | | | BCL2, GNAS, |
| | | | | | | | | | | IGF2R, IRS2, IRS2, |
| | | | | | | | | | | MAP2K1, NTRK3, |
| | | | | | | | | | | TGFBR2 |
| 301 | 016 | DLBCL | 23 | SD | | | | | | BRCA2, EPHA5, |
| | | | | | | | | | | GNAS, HSP90AA1, |
| | | | | | | | | | | INSR, NF1, PKHD1, |
| | | | | | | | | | | SMARCA4 |
| 401 | 003 | DLBCL | 35 | ND | CDKN2A, | | | | | ARID1A, CD79B, |
| | | | | | TP53 | | | | | CEBPA, EPHA5, |
| | | | | | | | | | | EPHA5, HSP90AA1, |
| | | | | | | | | | | IGF1R, MLH1, |
| | | | | | | | | | | NTRK1, SMO, TSC1 |
| 401 | 007 | DLBCL | 74 | SD | FBXW7, | RB1 | | | | ABL2, CHEK1, |
| | | | | | TP53 | | | | | ESR1, FLT1, MLL, |
| | | | | | | | | | | MUTYH, TSC1 |
| 401 | 008 | DLBCL | 56 | PD | APC, | | | | FBXW7 | APC, IGF2R, MLH1, |
| | | | | | DNMT3A, | | | | | MSH6 |
| | | | | | TNKS, | | | | | |
| | | | | | TP53 | | | | | |
| 402 | 005 | DLBCL | 57 | PD | | | | | | |
| 001 | 020 | GBM | 128 | Non | CDKN2A, | NF1 | FGFR3 | | | CD79A, CEBPA, |
| | | | | Prog | PIK3CA, | | | | | EPHB4, JAK2, |
| | | | | | TP53 | | | | | MSH6, PAX5, |
| | | | | | | | | | | PKHD1 |
| 001 | 022 | GBM | 136 | Non | BRAF, | | | | | EPHA6 |
| | | | | Prog | NF1, PTEN | | | | | |
| 001 | 024 | GBM | 21 | Non | | | EGFR | EGFR | CDKN2A, | ARID1A |
| | | | | Prog | | | | | CDKN2B, | |
| | | | | | | | | | PTEN | |
| 002 | 028 | GBM | 44 | PD | RB1, TP53 | | EGFR | EGFR | | ATR, CBL, ESR1, |
| | | | | | | | | | | GNAS, LTK, |
| | | | | | | | | | | PIK3CG |
| 007 | 001 | GBM | 62 | PD | | RB1 | | EGFR | | PIK3CG, PIK3R1, |
| | | | | | | | | | | RAFT, TSC2 |
| 007 | 002 | GBM | 22 | Prog | | | | EGFR | CDKN2A, | ATR, CDH5, LRP1B, |
| | | | | | | | | | CDKN2B | PDGFRA |
| 007 | 003 | GBM | 82 | PD | | | EGFR | EGFR | CDKN2A, | AR, BCL2A1, EGFR, |
| | | | | | | | | | CDKN2B | FLT3 |
| 007 | 004 | GBM | 41 | Non | EGFR, | | | EGFR | CDKN2A, | CEBPA, FLT3, |
| | | | | Prog | EGFR | | | | CDKN2B, | RPTOR |
| | | | | | | | | | PTEN | |
| 007 | 005 | GBM | 56 | Non | FGFR1, | | | | CDKN2A, | BRCA2, CD79B, |
| | | | | Prog | PKHD1 | | | | CDKN2B | CEBPA, CEBPA, |
| | | | | | | | | | | GPR124, MEN1, |
| | | | | | | | | | | NF1, PKHD1 |
| 007 | 006 | GBM | 23 | Non | | PIK3R1 | | | BRCA2, | PKHD1 |
| | | | | Prog | | | | | CDKN2A, | |
| | | | | | | | | | CDKN2B | |
| 007 | 007 | GBM | 46 | Non | EGFR | | | EGFR | CDKN2A, | AR, GPR124, |
| | | | | Prog | | | | | CDKN2B | LRP1B, NOTCH1, |
| | | | | | | | | | | PRKDC |
| 007 | 008 | GBM | 70 | Non | | | | | | |
| | | | | Prog | | | | | | |
| 007 | 010 | GBM | 56 | PD | EGFR, | BRCA2 | | EGFR | CDKN2A, | MLH1, MLL, |
| | | | | | PTEN | | | | CDKN2B | PLCG1, TSC1 |
| 001 | 018 | HCC | 110 | SD | | | | | | |
| 001 | 023 | HCC | 21 | NE/ND | | | | | | BAP1, CBL, CBL, |
| | | | | | | | | | | FLT4, JAK2, MLL, |
| | | | | | | | | | | PHLPP2, RICTOR, |
| | | | | | | | | | | TSC2 |
| 002 | 022 | HCC | 109 | PD | | | | | | |
| 002 | 023 | HCC | 112 | SD | CDKN2A, | | | | | APC, BRCA2, |
| | | | | | CTNNB1 | | | | | EPHB4, ERBB4, |
| | | | | | | | | | | JAK1, LRP1B, |
| | | | | | | | | | | PRKDC, TSC2 |
| 002 | 027 | HCC | 21 | NE/ND | | | | | | |
| 002 | 029 | HCC | 29 | NE/ND | TP53 | CTNNB1 | | | | DDR2, FANCA, |
| | | | | | | | | | | MAP2K4, RICTOR |
| 004 | 002 | HCC | 25 | NE (clin | TP53 | | ABL2 | | PTEN | BRAF, CDH1, |
| | | | | prog) | | | | | | MTOR, PAX5, |
| | | | | | | | | | | PDGFRA |
| 004 | 010 | HCC | 15 | NE | | | RB1 | | | CDH20, CDH5, |
| | | | | | | | | | | LRP1B, MRE11A, |
| | | | | | | | | | | RICTOR |
| 004 | 011 | HCC | 37 | ND | CTNNB1 | | | | | KRAS, NF1, PHLPP2 |
| 004 | 012 | HCC | 14 | ND | CTNNB1 | | | | | CHEK2, DOT1L, |
| | | | | | | | | | | ERBB3, NOTCH1, |
| | | | | | | | | | | NTRK1, PKHD1 |
| 004 | 016 | HCC | 36 | ND | | | | | | AURKB, MSH6, |
| | | | | | | | | | | NOTCH1, NTRK1, |
| | | | | | | | | | | NTRK1, PKHD1, |
| | | | | | | | | | | SMO, TSC1 |
| 004 | 020 | HCC | 77 | SD | CTNNB1 | | | | | CEBPA, GNAS, KIT, |
| | | | | | | | | | | MLL, NF1, SRC |
| 006 | 006 | HCC | 105 | PR | TP53 | ARID1A | | CCNE1 | | ALK, ATM, CEBPA, |
| | | | | | | | | | | ERBB4, FGFR3, |
| | | | | | | | | | | GPR124, GPR124, |
| | | | | | | | | | | MCL1, MLL, MLL, |
| | | | | | | | | | | MSH2, PIK3CG, |
| | | | | | | | | | | PKHD1, STK11 |
| 006 | 011 | HCC | 181 | PR | | | | MYC | CDKN2A, | ABL2, ARID1A, |
| | | | | | | | | | CDKN2B | ATR, BRCA1, |
| | | | | | | | | | | BRCA2, CEBPA, |
| | | | | | | | | | | ERCC2, GPR124, |
| | | | | | | | | | | IGF1R, IKZF1, |
| | | | | | | | | | | JAK2, MITF, RBI, |
| | | | | | | | | | | RICTOR, TP53 |
| 006 | 012 | HCC | 49 | ND | DNMT3A | NF2 | | | | BRCA2, CARD11, |
| | | | | | | | | | | CDK4, DOT1L, |
| | | | | | | | | | | EPHA5, FGFR1, |
| | | | | | | | | | | FLT4, IRS2, MDM2, |
| | | | | | | | | | | NPM1 |
| 006 | 014 | HCC | 14 | NE | | | | | | AR, JAK1, KDR |
| 006 | 015 | HCC | 103 | SD | | | | | | |
| 006 | 016 | HCC | 324 | SD | | TP53 | | | | CDK8, FLT4, |
| | | | | | | | | | | GPR124, IGF2R, |
| | | | | | | | | | | PAX5, PKHD1, |
| | | | | | | | | | | TNKS2 |
| 008 | 018 | HCC | 120 | SD | | TP53 | | | | EPHA7, FLT1, |
| | | | | | | | | | | LRP1B, LRP1B, |
| | | | | | | | | | | MSH6, NF1, PTCH1, |
| | | | | | | | | | | PTCH1, TSC1 |
| 009 | 001 | HCC | 28 | NE | | ATM, | | | | ALK, FGFR4 |
| | | | | | | FANCA | | | | |
| 009 | 003 | HCC | 164 | SD | CTNNB1 | | | | | BCL6, EPHA7, |
| | | | | | | | | | | JAK3, TSC2 |
| 009 | 004 | HCC | 34 | NE/ND | | | | | | |
| 201 | 008 | HCC | 52 | PD | | | | | | |
| 301 | 001 | HCC | 117 | SD | CTNNB1 | | | EGFR | | GPR124, MCL1, |
| | | | | | | | | | | MLH1, MYCL1, |
| | | | | | | | | | | PKHD1, PRKDC, |
| | | | | | | | | | | RPTOR |
| 302 | 001 | HCC | 112 | SD | | | | | | CDH2, CDH5, |
| | | | | | | | | | | DOT1L, FGFR3, |
| | | | | | | | | | | GPR124, MYCN, |
| | | | | | | | | | | NF2, NTRK2, |
| | | | | | | | | | | RICTOR, TSC1 |
| 001 | 028 | HCC | 74 | SD | FGFR1, | BRCA2, | | | | ALK, APC, |
| | | exp. | | | TP53, | IKZF1, | | | | APCDD1, ARID1A, |
| | | | | | TRRAP | NOTCH2 | | | | ASXL1, AURKA, |
| | | | | | | | | | | BCL6, BLM, |
| | | | | | | | | | | BRCA1, BRIP1, |
| | | | | | | | | | | CDC73, CHEK1, |
| | | | | | | | | | | CREBBP, CUL4A, |
| | | | | | | | | | | CUL4B, EGFR, |
| | | | | | | | | | | ERBB4, ERBB4, |
| | | | | | | | | | | ERG, FANCM, |
| | | | | | | | | | | FAT3, FAT3, FAT3, |
| | | | | | | | | | | FGF12, FGF7, |
| | | | | | | | | | | FGFR1, FGFR1, |
| | | | | | | | | | | GNAQ, GNAS, |
| | | | | | | | | | | GNAS, GNAS, |
| | | | | | | | | | | GRIN2A, GRIN2A, |
| | | | | | | | | | | IGF1R, IL7R, |
| | | | | | | | | | | INHBA, JAK1, |
| | | | | | | | | | | KDM5A, KDM5C, |
| | | | | | | | | | | KEAP1, KIT, |
| | | | | | | | | | | KLHL6, LRP1B, |
| | | | | | | | | | | LRP1B, LRP1B, |
| | | | | | | | | | | LRP1B, MAP2K4, |
| | | | | | | | | | | MAP3K13, MLL2, |
| | | | | | | | | | | MTOR, MTOR, |
| | | | | | | | | | | MYST3, NOTCH1, |
| | | | | | | | | | | NOTCH1, NOTCH1, |
| | | | | | | | | | | NOTCH3, NOTCH3, |
| | | | | | | | | | | NOTCH4, NOTCH4, |
| | | | | | | | | | | NOTCH4, NOTCH4, |
| | | | | | | | | | | NTRK2, NTRK2, |
| | | | | | | | | | | NTRK2, NTRK2, |
| | | | | | | | | | | PAK7, PAK7, PAK7, |
| | | | | | | | | | | PDGFRB, PIK3CG, |
| | | | | | | | | | | PIK3CG, PIK3CG, |
| | | | | | | | | | | PIK3R2, PNRC1, |
| | | | | | | | | | | PRKDC, PTCH1, |
| | | | | | | | | | | RAD51C, RPA1, |
| | | | | | | | | | | RPTOR, RUNX1T1, |
| | | | | | | | | | | RUNX1T1, SH2B3, |
| | | | | | | | | | | SMO, SMO, SYK, |
| | | | | | | | | | | TGFBR2, TIPARP, |
| | | | | | | | | | | TOP1, TRRAP, |
| | | | | | | | | | | TSHR |
| 001 | 031 | HCC | 117 | SD | TP53 | | | | | ABL1, ERBB2, |
| | | exp. | | | | | | | | FAT3, MAP3K1, |
| | | | | | | | | | | MET, MET, MET, |
| | | | | | | | | | | PRDM1, PRKDC |
| 001 | 032 | HCC | 49 | NE | TP53 | | ATM | CCND1, | | ALK, CDKN2C, |
| | | exp. | | | | | | FGF19, FGF3, | | CTNNB1 |
| | | | | | | | | FGF4 | | |
| 001 | 033 | HCC | 106 | SD | FGFR2 | | | MCL1 | | CDKN2A, MAP3K1, |
| | | exp. | | | | | | | | TOP1 |
| 001 | 034 | HCC | 279 | SD | MTOR, | | | MYC, MYST3 | | BRCA1, DNMT3A, |
| | | exp. | | | TSC2 | | | | | GNAS, MLL, |
| | | | | | | | | | | MYCL1, MYCL1, |
| | | | | | | | | | | NFE2L2, PRKDC, |
| | | | | | | | | | | PTEN, TRRAP |
| 001 | 035 | HCC | 114 | SD | PIK3CA | | | | | BRCA1, CCND1, |
| | | exp. | | | | | | | | MLL, NOTCH3, |
| | | | | | | | | | | PTCH1, PTCH1 |
| 001 | 036 | HCC | 48 | ND | CTNNB1 | | | MYC | | FANCE, FAT3, |
| | | exp. | | | | | | | | MAP3K1, MAP3K1, |
| | | | | | | | | | | NTRK2, NUP93, |
| | | | | | | | | | | NUP93 |
| 002 | 032 | HCC | 26 | SD | TP53 | | | MYC | | ATR, BRCA2, |
| | | exp. | | | | | | | | CTNNB1, DAXX, |
| | | | | | | | | | | GPR124, MLL, |
| | | | | | | | | | | PDGFRA, RPTOR |
| 002 | 036 | HCC | 57 | SD | ERBB4, | | FANCA | KDR, KIT, | | BRCA1, KDM5A, |
| | | exp. | | | TP53, TP53 | | | PDGFRA, | | KDM5A, MSH6, |
| | | | | | | | | RICTOR | | NOTCH2 |
| 002 | 037 | HCC | 203 | SD | MSH6, | ATRX, | | | | ARAF, ATR, ATR, |
| | | exp. | | | TP53 | FLT3, | | | | AXL, CARD11, |
| | | | | | | NOTCH3, | | | | CDK6, CIC, EPHB1, |
| | | | | | | RB1 | | | | FANCM, FAT3, |
| | | | | | | | | | | FLT1, FLT4, IKZF1, |
| | | | | | | | | | | KDR, MAP2K1, |
| | | | | | | | | | | MAP2K2, MEF2B, |
| | | | | | | | | | | MLL2, NF1, NF1, |
| | | | | | | | | | | NKX2-1, NOTCH1, |
| | | | | | | | | | | NSD1, NTRK2, |
| | | | | | | | | | | PAK7, PAK7, |
| | | | | | | | | | | PARP3, PARP4, |
| | | | | | | | | | | PDGFRA, PDGFRA, |
| | | | | | | | | | | PRKDC, PRKDC, |
| | | | | | | | | | | RBI, RUNX1T1, |
| | | | | | | | | | | RUNX1T1, |
| | | | | | | | | | | SMARCA4, SPEN, |
| | | | | | | | | | | SPOP, TSC1 |
| 002 | 038 | HCC | 314 | SD | | | | | | |
| | | exp. | | | | | | | | |
| 002 | 039 | HCC | 29 | NE | TP53 | ATM | | CCND1, | | APC, ATM, |
| | | exp. | | | | | | FGF19, FGF3, | | CARD11, GRIN2A, |
| | | | | | | | | FGF4 | | LRP1B, PARP4 |
| 003 | 020 | HCC | 45 | ND | | | | | | |
| | | exp. | | | | | | | | |
| 006 | 017 | HCC | 393 | PD | | | | MYC, SRC, | | ATM, MLL2, |
| | | exp. | | | | | | ZNF217 | | NOTCH1, NOTCH3 |
| 006 | 018 | HCC | 390 | SD | CTNNB1, | FAM123B | | CCNE1 | | BRAF, BRCA2, |
| | | exp. | | | TP53 | | | | | C11orf30, CDK12, |
| | | | | | | | | | | CEBPA, FAM123B, |
| | | | | | | | | | | FANCA, FAT3, |
| | | | | | | | | | | FAT3, FLT3, MLL2, |
| | | | | | | | | | | MSH2, MSH6, |
| | | | | | | | | | | SH2B3, TET2, |
| | | | | | | | | | | UGT1A7 |
| 006 | 019 | HCC | 309 | SD | | | | | | |
| | | exp. | | | | | | | | |
| 006 | 020 | HCC | on- | PR | AKT1, | ASXL1 | | | CDKN2A | ATRX, BRCA1, |
| | | exp. | going | | CTNNB1, | | | | | CREBBP, CTNNA1, |
| | | | | | KRAS | | | | | GPR124, IGF1R, |
| | | | | | | | | | | LRP1B, MAP3K13, |
| | | | | | | | | | | MITF, MSH2, |
| | | | | | | | | | | NTRK2, SETD2, |
| | | | | | | | | | | TNFAIP3, ZNF217 |
| 008 | 045 | HCC | 136 | SD | | | | | | |
| | | exp. | | | | | | | | |
| 009 | 011 | HCC | 56 | PD | FANCM | BACH1, | | MYC | | BLM, CDK4, |
| | | exp. | | | | BRCA1, | | | | GATA2, MTOR, |
| | | | | | | BRCA1 | | | | PARP1, SF3B1 |
| 009 | 012 | HCC | 8 | NE | | | | | | |
| | | exp. | | | | | | | | |
| 009 | 014 | HCC | 56 | PD | | | | | | |
| | | exp. | | | | | | | | |
| 009 | 019 | HCC | 56 | PD | TP53 | ARID2, | | | | CEBPA, CHUK, |
| | | exp. | | | | ARID2, | | | | KDM5A, LRP1B, |
| | | | | | | CSF1R | | | | MPL, PARP1, |
| | | | | | | | | | | SH2B3, TET2, |
| | | | | | | | | | | ZNF703 |
| 301 | 023 | HCC | 70 | SD | TP53 | | | MCL1 | | ATM, NF1, PTCH1, |
| | | exp. | | | | | | | | SOX10, TET2 |
| 301 | 024 | HCC | 113 | SD | ATM, | | | | | KDM5C, PARP2, |
| | | exp. | | | CTNNB1 | | | | | PARP4, PARP4, |
| | | | | | | | | | | PARP4, PRKDC, |
| | | | | | | | | | | RAD50 |
| 302 | 006 | HCC | 82 | SD | TP53 | | | | TSC2 | CREBBP, ERBB4, |
| | | exp. | | | | | | | | FAT3, KIT, RB1 |
| 401 | 010 | HCC | 337 | SD | | | | | | |
| | | exp. | | | | | | | | |
| 401 | 011 | HCC | 134 | SD | | | | | | AR, FAT3, INHBA, |
| | | exp. | | | | | | | | NOTCH2, RPTOR, |
| | | | | | | | | | | ZNF703 |
| 402 | 006 | HCC | 36 | NE | DNMT3A, | PTEN | | CCND1, | | AKT2, ERBB3, |
| | | exp. | | | MSH6, | | | FGF19, FGF3, | | FAT3, FGFR4, |
| | | | | | PTEN, | | | FGF4 | | FIP1L1, GNAS, |
| | | | | | TP53 | | | | | IGF2, IRS2, NF1, |
| | | | | | | | | | | NTRK2, PRKDC, |
| | | | | | | | | | | RAD51L3, TRRAP |
| 002 | 025 | MM | 21 | ND | | | | | | |
| 003 | 001 | MM | 35 | PD | | | | | | |
| 008 | 010 | MM | 12 | PD | | | | | | |
| 008 | 014 | MM | 28 | NE | | | | | | |
| 201 | 003 | MM | 76 | PD | TP53 | | | | | FGFR3, FGFR3, |
| | | | | | | | | | | PTPRD, PTPRD, |
| | | | | | | | | | | PTPRD |
| 202 | 001 | MM | 66 | SD | KRAS | | | | | AURKB, BCL6, |
| | | | | | | | | | | CDH20, LRP1B, |
| | | | | | | | | | | LRP1B, TET2 |
| 202 | 003 | MM | 42 | PD | KRAS | DNMT3A | | | | MCL1, NOTCH1 |
| 202 | 004 | MM | 7 | PD | PTPN11 | | | BRAF | RB1 | EPHB4, LRP6, |
| | | | | | | | | | | MYCN |
| 301 | 003 | MM | 127 | SD | KRAS | | | | | ATR, CHEK2, |
| | | | | | | | | | | DDR2, EPHB1, |
| | | | | | | | | | | IKBKE, KIT |
| 301 | 004 | MM | 90 | SD | | | | | | |
| 301 | 008 | MM | 348 | PD | | | | | | CDH20, CDH20, |
| | | | | | | | | | | CDH5, CDH5, |
| | | | | | | | | | | PKHD1, PKHD1, |
| | | | | | | | | | | PRKDC, PRKDC |
| 301 | 009 | MM | 43 | PD | BRAF | | | | | CDKN2A, INHBA, |
| | | | | | | | | | | LTK, PKHD1, RET, |
| | | | | | | | | | | RET, SMARCA4, |
| | | | | | | | | | | TSC1 |
| 301 | 012 | MM | 46 | SD | BRAF | | | | | CBL, CDKN2C, |
| | | | | | | | | | | HSP90AA1, LRP1B, |
| | | | | | | | | | | SMARCA4 |
| 301 | 013 | MM | 48 | PD | | | | | | |
| 001 | 025 | NET | 127 | SD | | | | | | IKBKE |
| 001 | 026 | NET | 319 | SD | | | | | | |
| 004 | 024 | NET | 17 | NE | EPHA7, | MEN1, | | AURKB | | FLT4, GNAS, TSC2 |
| | | | | | PTEN | SMARCA | | | | |
| | | | | | | 4 | | | | |
| 004 | 025 | NET | on- | SD | | | | | | |
| | | | going | | | | | | | |
| 008 | 016 | NET | 784 | SD | | | | | | |
| 008 | 017 | NET | on- | SD | | | | | | |
| | | | going | | | | | | | |
| 008 | 021 | NET | 539 | PR | | | | | | |
| 008 | 022 | NET | 603 | SD | | | | | | |
| 008 | 023 | NET | 21 | NE | | | | | | |
| 008 | 024 | NET | 262 | SD | | | | | | ALK, CEBPA |
| 008 | 025 | NET | on- | SD | | | | | | ARID1A, ARID1A, |
| | | | going | | | | | | | ESR1, JAK3, |
| | | | | | | | | | | NTRK1, PRKDC, |
| | | | | | | | | | | SMO |
| 008 | 027 | NET | 443 | SD | | | | | | |
| 008 | 031 | NET | 673 | SD | | | | | | |
| 008 | 032 | NET | on- | SD | | | | | | |
| | | | going | | | | | | | |
| 008 | 034 | NET | 420 | SD | | | | | | |
| 008 | 035 | NET | 252 | SD | | | | | | |
| 008 | 036 | NET | on- | SD | | | | | | ALK, AR, ARID1A, |
| | | | going | | | | | | | FOXP4, GNAS, |
| | | | | | | | | | | KDM6A, KDM6A |
| 008 | 037 | NET | on- | SD | | | | | | |
| | | | going | | | | | | | |
| 009 | 009 | NET | 50 | NE | | | | | | |
| 401 | 002 | NET | on- | SD | | | | | | JUN, MSH6, SMO |
| | | | going | | | | | | | |
| 401 | 004 | NET | 7 | NE | | | | | | |
| 401 | 005 | NET | 423 | SD | | | | | | |
| 401 | 006 | NET | 115 | SD | CDKN2A, | | | | | AR, GPR124 |
| | | | | | FGFR1 | | | | | |
| 001 | 029 | NET | 77 | SD | | | | | | |
| | | exp. | | | | | | | | |
| 002 | 033 | NET | 119 | SD | | | | | | |
| | | exp. | | | | | | | | |
| 002 | 035 | NET | 106 | SD | | | | | | |
| | | exp. | | | | | | | | |
| 003 | 019 | NET | 56 | PD | | | | | | |
| | | exp. | | | | | | | | |
| 008 | 039 | NET | 245 | SD | | | | | | |
| | | exp. | | | | | | | | |
| 008 | 040 | NET | 200 | SD | | | | | | |
| | | exp. | | | | | | | | |
| 008 | 042 | NET | 51 | SD | | | | | | |
| | | exp. | | | | | | | | |
| 008 | 043 | NET | on- | SD | | | | | | |
| | | exp. | going | | | | | | | |
| 008 | 044 | NET | 278 | SD | | | | | | |
| | | exp. | | | | | | | | |
| 008 | 046 | NET | on- | SD | | | | | | |
| | | exp. | going | | | | | | | |
| 008 | 047 | NET | 35 | ND | | | | | | |
| | | exp. | | | | | | | | |
| 008 | 048 | NET | 265 | SD | | | | | | |
| | | exp. | | | | | | | | |
| 008 | 049 | NET | on- | SD | | | | | | |
| | | exp. | going | | | | | | | |
| 008 | 050 | NET | 368 | SD | | | | | | |
| | | exp. | | | | | | | | |
| 008 | 051 | NET | on- | SD | | | | | | |
| | | exp. | going | | | | | | | |
| 008 | 052 | NET | on- | SD | | | | | | |
| | | exp. | going | | | | | | | |
| 008 | 053 | NET | 253 | SD | | | | | | |
| | | exp. | | | | | | | | |
| 009 | 010 | NET | 86 | NE | | | | | | |
| | | exp. | | | | | | | | |
| 009 | 013 | NET | on- | PR | | | | | | |
| | | exp. | going | | | | | | | |
| 009 | 015 | NET | 15 | ND | | | | | | |
| | | /exp. | | | | | | | | |
| 009 | 018 | NET | 180 | SD | | | | | | |
| | | exp. | | | | | | | | |
| 201 | 010 | NET | 56 | PD | | | | | | |
| | | exp. | | | | | | | | |
| 401 | 009 | NET | 42 | SD | | | | | | |
| | | exp. | | | | | | | | |
| 301 | 007 | NSCL+ | 299 | PR | | STK11 | | EGFR | | AKT1, AR, ARID1A, |
| | | | | | | | | | | BRCA2, CARD11, |
| | | | | | | | | | | CDKN2A, CDKN2A, |
| | | | | | | | | | | DDR2, EPHA6, |
| | | | | | | | | | | FBXW7, FBXW7, |
| | | | | | | | | | | FGFR2, FLT4, |
| | | | | | | | | | | INHBA, JAK3, |
| | | | | | | | | | | LRP1B, LRP6, |
| | | | | | | | | | | NTRK1, PDGFRB, |
| | | | | | | | | | | RICTOR, |
| | | | | | | | | | | SMARCA4, SUFU, |
| | | | | | | | | | | TBX22, TNFAIP3, |
| | | | | | | | | | | TOP1, TSC2 |
| 302 | 002 | NSCL+ | 55 | PD | KRAS, | ATM | | NKX2-1 | | AR, ATM, |
| | | | | | STK11, | | | | | HSP90AA1, IGF2R, |
| | | | | | TP53 | | | | | IKBKE, LRP1B, |
| | | | | | | | | | | LRP1B, LRP1B, |
| | | | | | | | | | | STK11 |
| 002 | 020 | NSCLC | 24 | NE | EGFR, | | | EGFR, MYC | CDKN2A, | AKT2, ERBB3, |
| | | | | | EGFR | | | | CDKN2B | GPR124, INSR, LTK, |
| | | | | | | | | | | TP53 |
| 002 | 021 | NSCLC | 164 | SD | | TNFAIP3 | | BCL2L1, | | CARD11, CDH20, |
| | | | | | | | | CCND1 | | FGFR1, GPR124, |
| | | | | | | | | | | IGF2R, KIT, LRP1B, |
| | | | | | | | | | | MLL, NF1, RARA |
| 004 | 005 | NSCLC | 155 | SD | | | | | | EPHA5, ERBB3, |
| | | | | | | | | | | FLT3, GPR124, |
| | | | | | | | | | | HOXA3, JAK3, |
| | | | | | | | | | | KDR, NOTCH1, |
| | | | | | | | | | | PRKDC |
| 004 | 006 | NSCLC | 56 | SD | KRAS | | | | | FLT1, LRP1B, |
| | | | | | | | | | | RARA, TOP1 |
| 004 | 008 | NSCLC | 28 | ND | TP53 | | | | | CEBPA, EPHA7, |
| | | | | | | | | | | GNAS, LRP1B, |
| | | | | | | | | | | MAP2K2, PKHD1, |
| | | | | | | | | | | PRKDC |
| 004 | 014 | NSCLC | 60 | PD | CDKN2A | | | | | ATR, DOT1L, |
| | | | | | | | | | | EPHA6, EPHB1, |
| | | | | | | | | | | EPHB1, ERBB3, |
| | | | | | | | | | | JAK3, MYC, |
| | | | | | | | | | | NOTCH1, NTRK1, |
| | | | | | | | | | | PRKDC, PRKDC, |
| | | | | | | | | | | RET |
| 004 | 017 | NSCLC | 35 | ND | | | | MCL1 | | APC, CARD11, |
| | | | | | | | | | | CARD11, FANCA, |
| | | | | | | | | | | HSP90AA1, KDM6A |
| 006 | 002 | NSCLC | 26 | NE | TP53 | | | ERBB2 | SMAD4 | ATM, ERBB4, |
| | | | | | | | | | | FANCA, KIT, MLL, |
| | | | | | | | | | | MLL, MUTYH, |
| | | | | | | | | | | MYC, NKX2-1, |
| | | | | | | | | | | TSC2 |
| 006 | 003 | NSCLC | 83 | SD | TP53 | APC | BRCA1 | NKX2-1 | | ATM, EPHB6, |
| | | | | | | | | | | GPR124, GUCY1A2, |
| | | | | | | | | | | LRP1B, PIK3CG, |
| | | | | | | | | | | PLCG1, SMO, |
| | | | | | | | | | | TNFAIP3 |
| 006 | 004 | NSCLC | 70 | SD | VHL | | | | | BAP1, CEBPA, |
| | | | | | | | | | | KDR, PRKDC, |
| | | | | | | | | | | TGFBR2 |
| 006 | 005 | NSCLC | 28 | NE | MAP2K1 | | | | | CEBPA, FANCA, |
| | | | | | | | | | | FLT4, GUCY1A2, |
| | | | | | | | | | | LTK, MAP2K2, |
| | | | | | | | | | | NTRK1, PKHD1, |
| | | | | | | | | | | PKHD1, TET2 |
| 006 | 007 | NSCLC | 126 | SD | ERBB2 | | | | | ATM, FLT4, MLL, |
| | | | | | | | | | | NKX2-1, NKX2-1, |
| | | | | | | | | | | PIK3R1, TSC2, |
| | | | | | | | | | | USP9X |
| 006 | 008 | NSCLC | 22 | NE | EGFR | | | MDM2 | | APC, ATM, FLT4, |
| | | | | | | | | | | IRS2, PKHD1, |
| | | | | | | | | | | TNFAIP3 |
| 006 | 009 | NSCLC | 120 | SD | TP53 | DNMT3A | | | | AKT3, ALK, ALK, |
| | | | | | | | | | | AR, CBL, CRKL, |
| | | | | | | | | | | EPHA3, EPHA5, |
| | | | | | | | | | | EPHB1, EPHB6, |
| | | | | | | | | | | EPHB6, FLT1, |
| | | | | | | | | | | GNAS, LRP1B, |
| | | | | | | | | | | LRP1B, MTOR, NF1, |
| | | | | | | | | | | NTRK1, NTRK2, |
| | | | | | | | | | | PDGFRB, SMO, |
| | | | | | | | | | | TET2, TSC2 |
| 006 | 010 | NSCLC | 28 | NE | KRAS, | | | KRAS, MCL1, | | APC, BCL2L2, |
| | | | | | TP53 | | | SRC | | BRCA1, CD79B, |
| | | | | | | | | | | CDK6, EPHA7, |
| | | | | | | | | | | GNAS, LRP1B, |
| | | | | | | | | | | MITF, NKX2-1, |
| | | | | | | | | | | NOTCH1, NTRK1, |
| | | | | | | | | | | PDGFRA, SMAD4 |
| 008 | 002 | NSCLC | 28 | PD | | | ERG | | TP53 | AURKB, MLL, |
| | | | | | | | | | | PRKDC |
| 008 | 003 | NSCLC | 76 | ND | BRAF, | | | | | APC, BRCA2, |
| | | | | | CDKN2A, | | | | | IGF2R, MLL, MSH2, |
| | | | | | TP53 | | | | | SMO |
| 008 | 012 | NSCLC | 140 | SD | TP53 | PTEN, | | | | FLT4, HSP90AA1, |
| | | | | | | RB1 | | | | JAK2, PAK3, |
| | | | | | | | | | | PKHD1, RB1, |
| | | | | | | | | | | STAT3 |
| 201 | 004 | NSCLC | 69 | SD | | PTEN | | FGFR1, | | ARID1A, FGFR1, |
| | | | | | | | | PIK3CA, | | FLT4, LRP1B, |
| | | | | | | | | SOX2 | | LRP1B, MSH2, |
| | | | | | | | | | | NOTCH1, NTRK3, |
| | | | | | | | | | | PHLPP2, PRKDC, |
| | | | | | | | | | | RAF1, SMARCA4 |
| 201 | 005 | NSCLC | 229 | SD | TP53 | SMARCA | | | | ALK, ARID1A, ATR, |
| | | | | | | 4 | | | | CEBPA, FLT1, |
| | | | | | | | | | | GNAS, GUCY1A2, |
| | | | | | | | | | | MLL, MSH2, NF2, |
| | | | | | | | | | | NTRK1, PIK3CG, |
| | | | | | | | | | | PKHD1, PTPRD, |
| | | | | | | | | | | SMARCA4 |
| 201 | 006 | NSCLC | 8 | ND | PIK3CA, | | | MET | | EPHA5, EPHA5, |
| | | | | | PIK3CA, | | | | | FANCA, FANCA, |
| | | | | | PTEN, | | | | | PIK3CG, PIK3CG, |
| | | | | | TP53, TP53 | | | | | PIK3R1, PIK3R1, |
| | | | | | | | | | | PKHD1, PKHD1, |
| | | | | | | | | | | PTCH2, PTCH2, |
| | | | | | | | | | | USP9X, USP9X |
| 201 | 007 | NSCLC | 19 | ND | ARID1A | ARID1A | | MCL1, | | ABL2, ATM, |
| | | | | | | | | PIK3CA, | | EPHA5, EPHB6, |
| | | | | | | | | RICTOR, | | FGFR1, FGFR2, |
| | | | | | | | | SOX2 | | GNAS, IGF2R, |
| | | | | | | | | | | LRP1B, MET, MLL, |
| | | | | | | | | | | MLL, MLL, MTOR, |
| | | | | | | | | | | PIK3CG, PKHD1, |
| | | | | | | | | | | PRKDC, TGFBR2 |
| 301 | 005 | NSCLC | 170 | SD | | TP53 | | | | EPHA5, EPHB6, |
| | | | | | | | | | | ERG, NF1 |
| 301 | 006 | NSCLC | 58 | NE | | | | | | |

As can be seen in Table 2 and Table 3, for certain patients showing a tumor response (PR or SD) upon treatment with Compound 1, variants in one or more genes were shown.

### 6.5 Clinical Study C

A Phase 1b, Multi-Center, Open-Label Study of the TOR Kinase Inhibitor Compound 1 in Combination with Erlotinib or Oral Azacitidine in Advanced Non-Small Cell Lung Cancer. This Study is a Phase 1b, Multi-Center, Open-Label Study of the TOR Kinase Inhibitor Compound 1 in Combination with Erlotinib or Oral Azacitidine in Advanced Non-Small Cell Lung Cancer.

The primary objectives of the study are to determine the safety and tolerability of Compound 1 when administered orally in combination with either erlotinib or oral azacitidine and to define the non-tolerated dose (NTD) and the maximum tolerated dose (MTD) of each combination using NCI CTCAE v4; and to characterize the pharmacokinetics (PK) of Compound 1 and azacitidine following oral administration as single agents and after combination treatment. The secondary objectives of the study are to evaluate the effect of study drugs on mTORC1 and mTORC2 pathway biomarkers in blood and tumor; provide information on the preliminary efficacy of each drug combination; and characterize the PK of Compound 1 M1 metabolite after oral administration of Compound 1 as a single agent and in combination with erlotinib or oral azacitidine.

This is a clinical study of Compound 1 administered orally in combination with either oral erlotinib or oral azacitidine in subjects with Stage IIIB/IV NSCLC who have failed at least one line of standard therapy. It is a Phase 1b dose escalation and expansion study evaluating escalating dose levels of Compound 1 in combination with two dose levels of erlotinib (Arm A) or two dose levels of oral azacitidine administered either concurrently with Compound 1 (Arm B), or sequentially with Compound 1 (Arm C), followed by expansion of each combination cohort at one or more selected doses.

In Arm A, cohorts will receive escalating continuous daily doses (15 mg, 30 mg, and 45 mg) of Compound 1 in capsules concurrently with at least two different daily dose levels of erlotinib tablets (100 mg and 150 mg) in 28 day cycles after an initial single dose of Compound 1 seven days before, and a single dose of erlotinib on the first day of, the first cycle.

In Arm B, cohorts will receive escalating continuous daily dose levels of Compound 1 (15 mg, 30 mg, and 45 mg) concurrently with one or more dose levels of oral azacitidine (200 mg or 300 mg, as two or three 100 mg tablets) administered on Day 1 to 21 of each 28-day cycle after an initial single dose of Compound 1 seven days before, and a single dose of oral azacitidine on the first day of, the first cycle.

In Arm C, cohorts will receive escalating daily dose levels of Compound 1 (15 mg, 30 mg, and 45 mg) administered on Day 8 to 28 after one or more dose levels of oral azacitidine (200 mg or 300 mg, as two or three 100 mg tablets) administered on Day 1 to 7 of each 28-day cycle after an initial single dose of Compound 1 seven days before the first cycle.

A standard “3+3” dose escalation design will be used to identify initial toxicity of each combination. Subjects will be assigned to study treatment arms based on Investigator choice and open slots. Cohorts of 3 subjects will take study drugs in defined dose increments and, in the event of dose-limiting toxicity (DLT) in 1 of 3 evaluable subjects, cohorts will be expanded to 6 subjects.

An evaluable subject for DLT is defined as one that received at least 20 of the 27 planned doses of Compound 1, and 21 of the 28 planned doses of erlotinib, during Cycle 1 in Arm A; received at least 20 of the 27 planned doses of Compound 1, and 14 of 21 planned doses of oral azacitidine, during Cycle 1 in Arm B; received at least 14 of 21 planned doses of Compound 1, and 6 of 7 planned doses of oral azacitidine, during Cycle 1 in Arm C; experienced study drug-related DLT after receiving at least one dose.

Non-evaluable subjects not due to DLT will be replaced. Additional subjects within any dose cohort may be enrolled at the discretion of the Safety Review Committee (SRC).

A dose will be considered the NTD when 2 of 6 evaluable subjects in a cohort experience drug-related DLT in Cycle 1. The MTD is defined as the last dose level below the NTD with 0 or 1 out of 6 evaluable subjects experiencing DLT during Cycle 1. If 2 of 6 DLT are observed at the first dose level with either combination, a lower dose combination may be explored at the discretion of the SRC. An intermediate dose of Compound 1 (one between the NTD and the last dose level before the NTD) may be evaluated to accurately determine the MTD of the combination.

Following completion of dose escalation, each combination treatment arm will be expanded with approximately 10 additional evaluable subjects. Expansion may occur at the MTD established in the dose escalation phase, or at an alternative tolerable combination dose level, based on the review of safety, PK and PD data.

Tumor biopsy for analysis of genetic mutations and biomarkers of treatment activity is optional in the dose escalation phase but mandatory during the dose expansion phase. Paired tumor biopsies to evaluate tumor biomarkers of Compound 1, erlotinib and/or oral azacitidine activity will be required in the expansion cohorts.

The study population will consist of men and women, 18 years or older, with Stage IIIB/IV NSCLC, with disease progression following at least one standard first-line treatment regimen. First-line treatment may include either chemotherapy or an EGFR inhibitor.

Enrollment is expected to take approximately 15 months (9 months for dose escalation, 6 months for expansion). Completion of active treatment and post treatment follow-up is expected to take 6-12 additional months.

Dose levels to be explored in this Phase 1b study are shown below.

| | | | Arm B and C | |
|---|---|---|---|---|
| | Arm A | | Cmdp 1 (mg) | Oral Azacitidine (mg) |
| Dose | Cmpd 1 | Erlotinib | Arm B: D-7, D2-28 | Arm B: D1-21 |
| Level | (mg daily) | (mg daily) | Arm C: D-7, D8-28 | Arm C: D1-D7 |
| 1 | 15 | 100 | 15 | 200 |
| 2a | 15 | 150 | 15 | 300 |
| 2b | 30 | 100 | 30 | 200 |
| 3a | 30 | 150 | 30 | 300 |
| 3b | 45 | 100 | 45 | 200 |
| 4 | 45 | 150 | 45 | 300 |

If unacceptable toxicity occurs at dose level 1, only one dose reduction for each drug is allowed: Compound 1 10 mg, erlotinib 75 mg, and oral azacitidine 100 mg.

Dose levels 2a and 2b and dose levels 3a and 3b have comparable dose intensity and may be enrolled concurrently.

Treatment is administered in 28-day cycles. Compound 1 and erlotinib will be dosed daily in Arm A; oral azacitidine will be dosed concurrent with daily Compound 1 for the first 21 of 28 days in Arm B; oral azacitidine will be dosed only for 7 days before dosing with Compound 1 alone for 21 of 28 days in Arm C. For both the dose escalation and expansion phases, slight modifications to the dosing schedule will occur prior to and during Cycle 1 in order to facilitate PK and PD evaluation of each drug alone and in combination. Administration of study drugs is described below:
In Arm A, B and C:
   One week (Day −7) prior to Cycle 1, a single dose of Compound 1 will be administered followed by PK and PD sampling.
In Arm A:
   During Cycle 1, a single oral dose of erlotinib will be administered on Day 1. Combined administration with Compound 1 will start on Day 2 and continue through Day 28.
   Starting with Cycle 2 and thereafter, both drugs will start on Day 1 and continue through Day 28.
In Arm B:
   During Cycle 1, a single dose of oral azacitidine will be administered on Day 1. Combined administration with Compound 1 will start on Day 2. Oral azacitidine will continue through Day 21 and Compound 1 through Day 28.
   Starting with Cycle 2 and thereafter, both drugs will start on Day 1. Oral azacitidine will continue through Day 21 and Compound 1 through Day 28.
In Arm C:
   During all cycles, oral azacitidine will be administered on Day 1 through 7 and Compound 1 will be administered on Day 8 through 28.

After the first dose is administered on Day 1 in any cohort, subjects will be observed for at least 28 days before the next higher protocol-specified dose cohort can begin. Intra-subject dose escalation of study drugs is not permitted during Cycle 1 but may be permitted in cycles beyond Cycle 1 if approved by the SRC. Dose reduction and temporary interruption of one or both drugs due to toxicity is allowed, but dose reduction during Cycle 1 will constitute DLT.

Study drugs are taken together at approximately the same time each morning. Due to a significant interaction of erlotinib with food, subjects in Arm A must take study drugs on an empty stomach at least 1 hour before and 2 hours after eating. There are no such food restrictions for subjects taking Compound 1 or oral azacitidine in Arms B and C.

Study treatment may be discontinued if there is evidence of disease progression, unacceptable toxicity or subject/physician decision to withdraw. Subjects may continue to receive study drugs beyond disease progression at the discretion of the Investigator.

The estimated total number of subjects to be enrolled during dose escalation is 54 to 108, depending on cohort size. Approximately 30 additional subjects (10 per regimen) will be evaluated for safety, PK, PD and preliminary antitumor effects during the expansion phase.

Subjects will be evaluated for efficacy after every 2 cycles through Cycle 6 and every 3 cycles thereafter. All treated subjects will be included in the efficacy analysis. The primary efficacy variable is tumor response rate and by progression-free survival at the end of 4 cycles of treatment. Tumor response will be determined by the Investigator, based on Response Evaluation Criteria in Solid Tumors (RECIST 1.1; Eisenhauer E. A., Therasse P., Bogaerts J., et al. New response evaluation criteria in solid tumours: Revised RECIST guideline (version 1.1). European J. Cancer; 2009; (45) 228-247)).

Secondary and exploratory endpoints include evaluation of mTOR, EGFR, and oral azacitidine biomarkers in blood and/or tumor and exploration of PK, PD, toxicity, and activity relationships.

The safety variables for this study are adverse events (AEs), safety clinical laboratory variables, 12-lead electrocardiograms (ECGs), left ventricular ejection fraction (LVEF) assessments, physical examinations, vital signs, exposure to study treatment, assessment of concomitant medications, and pregnancy testing for females of child bearing potentials (FCBP).

During dose escalation, the decision to either evaluate a higher dose level or declare an MTD will be determined by the SRC, based on their review of all available clinical and laboratory safety data for a given dose cohort.

The SRC will also select the dose and schedule of Compound 1 in combination with erlotinib and oral azacitidine appropriate for cohort expansion. One or both schedules of Compound 1 and oral azacitidine may be selected for cohort expansion. The SRC will continue to review safety data regularly throughout the study and make recommendations about study continuation and dose modification, as appropriate.

The concentration-time profiles of Compound 1, M1, erlotinib and oral azacitidine will be determined from serial blood samples collected after administration of study drugs as single agents and after combination treatment. The pharmacokinetics (PK) of Compound 1 and azacitidine will be determined after oral administration of each drug as a single agent and after combination treatment (Compound 1/oral azacitidine) using: (1) Maximum observed concentration in plasma (Cₘₐₓ), (2) Area under the concentration-time curve (AUC), (3) Time to maximum concentration (tₘₐₓ), (4) Terminal half-life (T_{1/2}), (5) Apparent total body clearance (CL/F) and (6) Apparent volume of distribution (Vz/F).

The effect of erlotinib and oral azacitidine on Compound 1 and M1 PK will be assessed, as will the effect of Compound 1 on the PK of erlotinib and oral azacitidine. Systemic exposure of Compound 1 after administration of Compound 1 as a single agent and in combination with erlotinib or oral azacitidine will be correlated with safety, PD and activity outcomes. The principal metabolites of Compound I, including M1, will be quantified in plasma. The PK of the M1 metabolite after oral administration of Compound I as a single agent and in combination with erlotinib or oral azacitidine will be characterized.

Biomarker evaluation will include analysis of mTOR pathway biomarkers, and other signaling pathways when possible, in blood and tumor after both single agent and combination treatment. In some instances, the changes of each biomarker will be determined by comparing the levels of biomarkers in pre- and on-treatment samples and, where possible, correlate these with PK findings and tumor response over time.

Assessment of gene DNA methylation and expression status in blood and tumor (when available) will be assessed at baseline and during combination drug treatment in Arm B and C to explore potential predictors of sensitivity to the Compound 1 plus oral azacitidine combination and effect of combination treatment on DNA methylation and expression.

Tumor gene sequencing will be performed at baseline on archival or Screening tumor biopsies to test for multiple genomic abnormalities.

Inclusion criteria for the study are: (1) Men and women, 18 years or older, with histologically or cytologically-confirmed, Stage IIIB/IV Non-Small Cell Lung Cancer with tumor progression following at least one prior treatment regimen (either chemotherapy or an Epidermal Growth Factor Receptor inhibitor for advanced disease), (2) Eastern Cooperative Oncology Group Performance Score of 0 or 1, (3) the following laboratory values: Absolute Neutrophil Count (ANC)≧1.0×10⁹/L; hemoglobin (Hgb)≧9 g/dL; platelets (plt)≧100×10⁹/L; potassium within normal limits or correctable with supplements; AST/SGOT and ALT/SGPT≦2.5× Upper Limit of Normal (ULN) or ≦5.0×ULN if liver tumor is present; serum bilirubin ≦1.5×ULN; estimated serum creatinine clearance of ≧60 mL/min/1.73 m² using the Cockcroft-Gault equation; subjects who complete Cycle 1 must meet the following hematologic criteria at the beginning of each subsequent cycle: ANC>1.0×10⁹/L; and platelets >75×10⁹/L.; and if the hematologic criteria are not met, the start of oral azacitidine in subsequent cycles may be delayed for up to 7 days to allow recovery. If recovery has not occurred after 7 days, this will be considered a DLT, (4) Adequate contraception (if appropriate), (5) Consent to retrieve archival tumor tissue, and (6) Consent to repeated tumor biopsy (dose expansion phase)

Exclusion criteria for the study are: (1) Prior systemic cancer-directed treatments or investigational drugs within 4 wks or 5 half lives, whichever is shorter, (2) Symptomatic central nervous system metastases, (3) Known acute or chronic pancreatitis, (4) Subjects with persistent diarrhea or malabsorption ≧NCI CTCAE grade 2, despite medical management, (5) Impaired cardiac function or significant cardiac disease, including any of the following: LVEF<45% as determined by MUGA or ECHO; complete left bundle branch or bifascicular block; congenital long QT syndrome; persistent or clinically meaningful ventricular arrhythmias; QTcF>460 msec on Screening ECG (mean of triplicate recordings); unstable angina pectoris or myocardial infarction ≦3 months prior to starting study drugs; uncontrolled hypertension (blood pressure ≧160/95 mmHg); (6) Diabetes on active treatment with either of the following: Fasting blood glucose (FBG)>126 mg/dL (7.0 mmol/L) or HbA1c≧6.5%, (7) Known Human Immunodeficiency Virus infection, chronic active hepatitis B or C virus infection, (8) Prior treatment with an investigational dual TORC1/TORC2, PI3K, or AKT inhibitor, (9) Major surgery ≦2 weeks prior to starting study drugs; no specific wash out is required for radiotherapy. Subjects must have recovered from any effects of recent therapy that might confound the safety evaluation of study drug, (10) Women who are pregnant or breast feeding. Adults of reproductive potential not employing two forms of birth control, and (11) history of concurrent second cancers requiring ongoing systemic treatment.

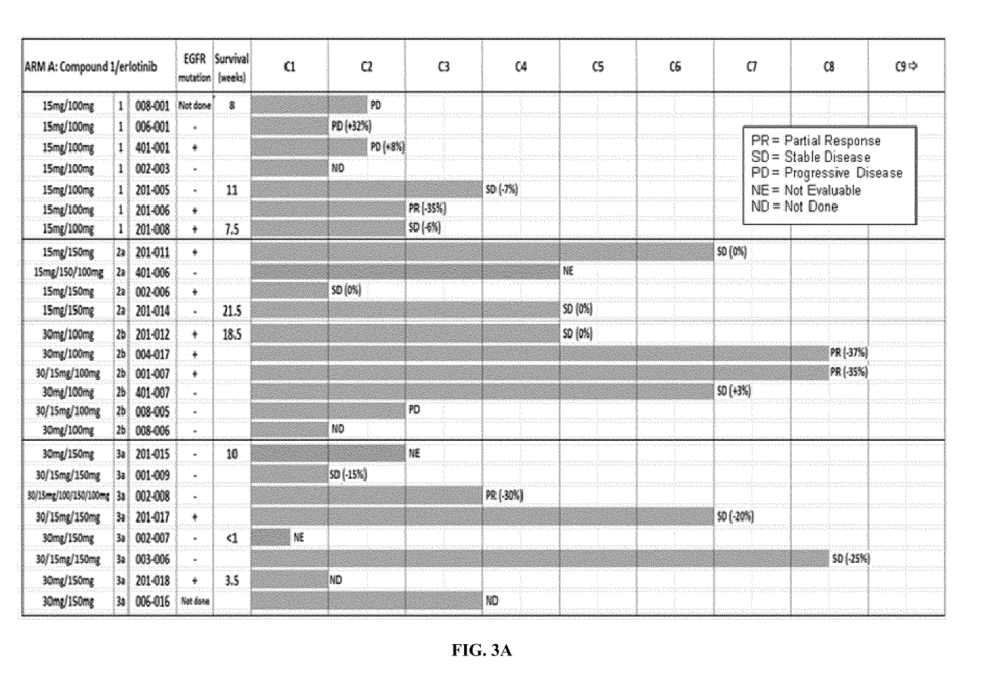

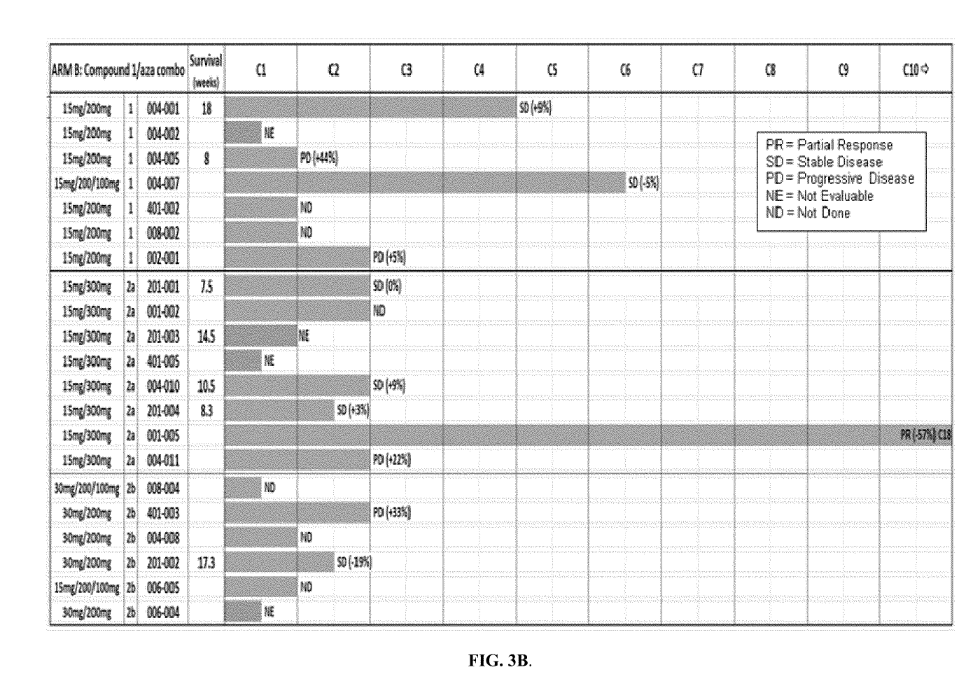

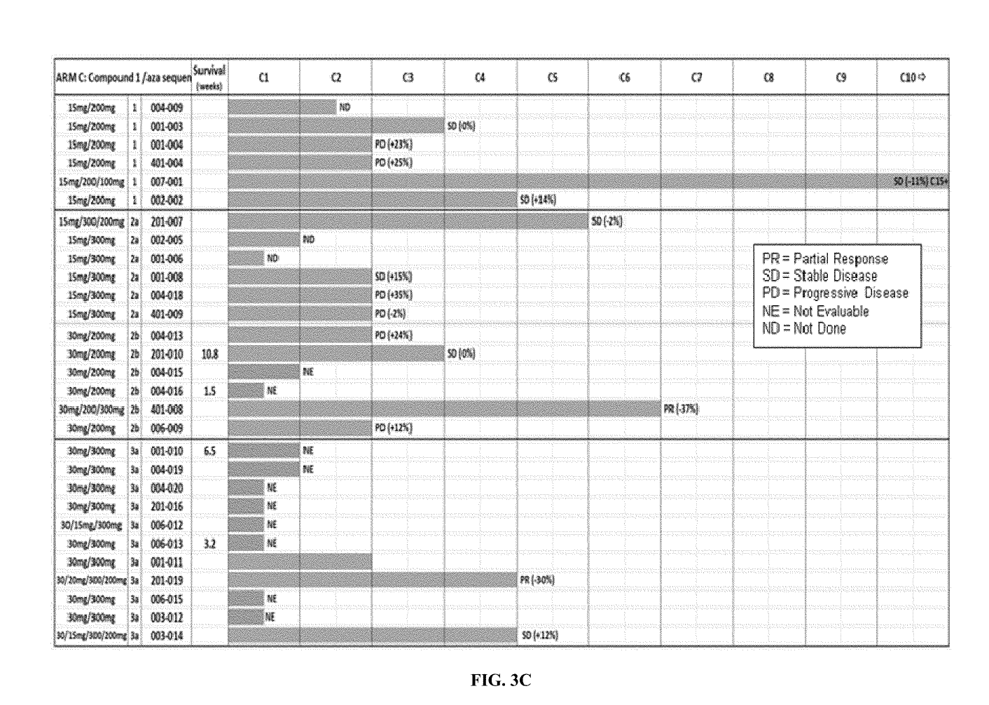

In some embodiments, patients undergoing the clinical protocol provided herein have shown, or will show a positive tumor response, such as inhibition of tumor growth or a reduction in tumor size. In certain embodiments, patients undergoing the clinical protocol provided herein achieved, or will achieve a Response Evaluation Criteria in Solid Tumors (for example, RECIST 1.1) of complete response, partial response or stable disease after administration of an effective amount of compound 1 in combination with an effective amount of erlotinib or oral azacytidine. In certain embodiments, patients undergoing the clinical protocol provided herein have shown or will show increased survival without tumor progression. In some embodiments, patients undergoing the clinical protocol provided herein have shown or will show inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), and/or increased Overall Survival (OS), among others. Patient disposition for Arm A, B and C is shown in FIGS. 3A, B and C.

Mutational analysis of the clinical samples was performed as described above in section 6.3, using the Foundation Medicine custom artifact databases from 2014 (Table 4).

Table 4 legend: Response assessment=best overall RECIST response; PR=partial response; SD=stable disease; PD=progressive disease; NE=non-evaluable; ND=not done. *: Confirmed reponse. Genes listed multiple times indicate detection of multiple mutations at different locations within the same gene.

As can be seen in Table 4, for certain patients showing a tumor response (PR or SD) upon treatment with Compound 1, variants in one or more genes were shown. No likely somatic variants, rearrangements, or deletions were observed in the subset of patients.

TABLE 4
Variants detected using Foundation Medicine custom artifact database from 2014

| | | | | | | Localized- | | |
|---|---|---|---|---|---|---|---|---|
| | | | | | | variants: | | |
| | | | Days | | Best | known- | Copy-number- | |
| | | | on | Combination - | overall | somatic- | variants: | Localized-variants: variants-of- |
| Site | Pt# | Tumor | Study | Dosing | response | variants | amplifications | unknown-significance |
| 002 | 8 | NSCLC | 90 | Cmpd 1 - Erlotinib | PR | TP53, | RICTOR, CDK6 | BRAF, CDK6, EPHA5, FANCM, |
| | | | | 30/15 mg- | | KDM5C, | | FGFR2, FGFR3, IRS2, KDR, KEAP1, |
| | | | | 100/150/100 mg | | STK11, | | MLL2, NOTCH4, TRRAP |
| | | | | | | LRP1B, | | |
| | | | | | | CDKN2A/B | | |
| 004 | 17 | NSCLC | 113 | Cmpd 1 - Erlotinib | PR | FAM123B, | MCL1, EGFR | ATR, BCL6, ERBB4, FAT3, FGFR1, |
| | | | | 30 mg-100 mg | | SMARCD1, | | FLT1, FLT4, GPR124, KEAP1, |
| | | | | | | TP53, | | LRP1B, MLL2, MYCN, NUP93, |
| | | | | | | ARID2 | | PIK3CG, IK3R2, RAD51C, RARA, |
| | | | | | | | | RET, TIPARP, GSK3B, GATA2, ATR, |
| | | | | | | | | TIPARP |
| 007 | 1 | NSCLC | 72 | Cmpd 1/Aza seq. | SD | ROS1 | | ALOX12B, CDH1, FAT3, FGF4, |
| | | | | 15 mg-200/100 mg | | | | FGF6, JAK1, MLL, NSD1, TSC1, |
| | | | | | | | | IL7R |
| 201 | 15 | NSCLC | 50 | Cmpd 1 - Erlotinib | NE | TP53, | BCL2L2, | AKT3, ALK, BLM, CASP8, EPHA5, |
| | | | | 30 mg-150 mg | | KDM5C, | NFKBIA, | FANCE, FAT3, INHBA, LRP1B, |
| | | | | | | STK11 | NKX2-1, | MTOR, NCOR1, NOTCH4, PDGFRA, |
| | | | | | | | RICTOR, HGF, | PDGFRB, PRKDC, SETD2, PIK3C3, |
| | | | | | | | MYC, ZNF703, | SMAD2, SMAD4, BCL2, IL7R, |
| | | | | | | | FGFR1, MYST3 | GPR124, PRKDC, NBN, RUNX1T1 |
| 401 | 8 | NSCLC | 173 | Cmpd 1/Aza seq. | PR | KRAS, | | ERBB4, IRF4, LRP1B, MLL2, |
| | | | | 30 mg-200/300 mg | | KEAP1, | | NTRK1, PDGFRA, SOCS1, TBX3, |
| | | | | | | STK11 | | TET2 |

### 6.6 Pharmacogenomic Analysis to Assess Correlation Between Efficacy Endpoints and Mutation Status of Genes or Gene Clusters

Pharmacogenomic tumor samples were collected for subjects enrolled into Part B. DNA was extracted from pre-treatment tumor samples and submitted for next generation sequencing as described above. A gene was considered to be mutant (variant) if it showed one of the following: mutation(s), for example, likely or known somatic variants; variants of unknown significance; or structural variation (deletion, amplification or rearrangement). A gene was considered to be wild type when no sequencing alterations are detected for this gene. A gene cluster is considered to be mutated if any gene in the cluster is mutated as defined above; otherwise the gene cluster is considered to be wild type. Sequence analysis was performed for the genes listed in FIG. 2.

Relationship Between Response and Gene Mutation Status.

Responses (PR and CR) assessed by the investigator using RECIST or IWC were tabulated by mutation status for genes of interest and tumor type. Similar tabulation was also provided for different malignancy groupings. When there were at least 3 responders observed in a tumor type, a Fisher's exact test was conducted to examine independence between these two variables. Raw p-values of such exact tests and its false discovery rate adjustment (adjusted across all genes tested) were provided. Without adjusting for multiplicity, a p-value (raw value) less than 0.05 is thought to mean that the response status is correlated with the mutation status for this particular gene.

Relationship Between Disease Control and Gene Mutation Status in Solid Tumor Cohorts.

In solid tumor cohorts, disease control status (whether subjects achieves best overall response as PR, CR, and SD) was tabulated by mutation status for genes of interest. When there are at least 3 disease control subjects observed in a tumor type, a Fisher's exact test was conducted to examine independence between these two variables. Raw p-values of such exact tests and its false discovery rate adjustment (adjusted across all genes tested) were provided. Without adjusting for multiplicity, a p-value (raw value) less than 0.05 is thought to mean that the response status is correlated with the mutation status for this particular gene.

Relationship Between Progression Free Survival and Gene Mutation Status.

Progression-Free Survival (PFS) was calculated as the time from first dose date to disease progression or death, whichever occurred first. Disease progression is determined by RECIST Version 1.1 criteria for solid tumor subjects, and IWC criteria for DLBCL. Within a tumor type and for selected genes, the Kaplan-Meier estimate of median PFS with its two-sided 95% CI was provided for each mutation group (mutant versus wild type) of that given gene. The Kaplan-Meier plots of progression free survival by cohort were presented. The raw P-value of the log rank test comparing survival distribution of PFS between mutant and wild type was provided.

PFS Censoring Details.

A subject who neither progressed nor died will be censored on the date of his or her last adequate tumor assessment. Subjects without valid baseline or post baseline tumor assessments will be censored on their first dose dates. Any valid per protocol tumor measurements for both target and non-target lesions are considered adequate.

Relationship Between Overall Survival and Gene Mutation Status in HCC Cohort.

In HCC cohort, overall survival (OS) is defined as the time from first dose to death. All deaths, regardless of the cause of death, will be included. Subjects who has no death reported will be censored at the last contact date the subject is known to be alive or the clinical cut-off date whichever is earlier.

In HCC cohort, the Kaplan-Meier estimates at the time points of 6 and 12 months were or will be provided for each mutation group (mutant versus wild type) of a given gene. The median of overall survival along with its two-sided 95% CI will be estimated. The Kaplan-Meier plots of overall free survival for each mutation group were presented. The raw P-value of the log rank test comparing survival distributions between mutant and wild type were provided.

Relationship Between Tumor Shrinkage and Mutation Status.

The relationship between tumor shrinkage and mutation status was assessed. Tumor shrinkage in solid tumor cohorts (except GBM) and DLBCL cohort was measured by best percent change from baseline in tumor size.

A Wilcoxon-Mann-Whitney test was conducted to compare tumor shrinkage between wild type and mutant subjects for selected genes within a given tumor type. The raw p-value of the Wilcoxon test was provided. Genes with corresponding p-values <0.05 were noted.

Relationship Between Total of SUV and Mutation Status.

The relationship between best percent change in total of SUV and mutation status was assessed. A Wilcoxon-Mann-Whitney test was conducted to compare best percent change in total of SUV between wild type and mutant subjects for selected genes. The raw p-value of the Wilcoxon test was provided. Genes with corresponding p-values <0.05 were noted.

DNA sequencing data are regarded as baseline characteristics and they are considered not to change after treatment. The endpoint are binary defined as wild type (WT) or mutated (MUT). A gene is considered to be “WT’ when no mutation is detected for this gene. A gene is considered to be “MUT” if it has structure variant (SV, copy number variation or rearrangement), no matter whether it has localized variant(s) and what type(s) of localized variant(s) it has. If a gene has ONLY localized variant(s), then 3 scenarios are considered: (a) known somatic variants only: a gene is considered to be “MUT” as long as it has known somatic variant(s), no matter whether it has likely somatic variants and/or variants of unknown significance or not; if it ONLY has likely somatic variant(s) or variant(s) of unknown significance, it is considered to be “WT”; (b) known+Likely variants: a gene is considered to be “MUT” when it has known or likely somatic variant(s), no matter whether it has variants of unknown significance or not; if it ONLY has variant(s) of unknown significance, it is considered to be “WT”; (c) all variants: a gene is considered to be “MUT” when it has known or likely somatic variant(s) or variant(s) of unknown significance. In addition, gene clusters will be considered for above three scenarios. Only the first two scenarios are considered in the exploratory analysis, as the last scenario has been reported in previous sections.

Based on statistical analysis described above, it is thought that genes with a raw p-value <0.05 are considered correlated with a given efficacy endpoint without adjustment for multiplicity.

In one embodiment, variants in the following genes are associated with response status accessed through RECIST or IWC. For HCC, variants in ARID1A, and/or CEBPA are associated with response status accessed through RECIST or IWC. For solid tumors, variants in one or more of ARID1A, FGFR2, IGF1R, RICTOR, and STK11 are associated with response status accessed through RECIST or IWC.

In one embodiment, variants in the following genes are associated with disease control status accessed through RECIST or IWC. For HCC, variants in GPR124 are associated with disease control status accessed through RECIST or IWC. In solid tumors, variants in GPR124 are associated with disease control status accessed through RECIST or IWC.

In one embodiment, variants in the following genes are associated with target lesion tumor shrinkage. For NSCLC, variants in TNFAIP3 are associated with target lesion tumor shrinkage.

In one embodiment, variants in the following genes are associated with PFS. For NSCLC, variants in one or more of APC, ARID1A, CARD11, FANCA, and KIT are associated with PFS. For DLBCL, variants in JAK2 are associated with PFS.

In one embodiment, variants in BRAF are associated with PFS.

6.7 Biomarkers Associated with Compound 1 Response in Hematological Cancers.

Cell Lines and Culture Conditions.

40 hematological cancer cell lines (Table 5) used in the study were purchased commercially. A lung cancer cell line A549 was used as a control cell line. A549 was purchased from National Cancer Institute (NCI) and cultured in RPMI+10% fetal bovine serum (FBS).

TABLE 5
Hematological Cancer Cell Lines

| Cell Line | Source | Catalog # | Growth Media |
|---|---|---|---|
| DB | DSMZ | ACC 539 | RPMI 1640 + 10% FBS |
| DOHH-2 | DSMZ | ACC 47 | RPMI 1640 + 10% FBS |
| Farage | ATCC | CRL-2630 | RPMI 1640 + 10% FBS |
| Granta-519 | DSMZ | ACC 342 | RPMI 1640 + 10% FBS |
| HL-60 | NCI | 502350 | RPMI 1640 + 10% FBS |
| HT | DSMZ | ACC 567 | RPMI 1640 + 10% FBS |
| JEKO-1 | DSMZ | ACC 553 | RPMI 1640 + 10% FBS |
| JVM-13 | ATCC | CRL-3003 | RPMI 1640 + 10% FBS |
| JVM-2 | DSMZ | ACC 12 | RPMI 1640 + 10% FBS |
| KARPAS- | DSMZ | ACC 545 | RPMI 1640 + 10% FBS |
| 1106P | | | |
| KARPAS- | DSMZ | ACC 31 | RPMI 1640 + 10% FBS |
| 299 | | | |
| KARPAS- | DSMZ | ACC 32 | RPMI 1640 + 10% FBS |
| 422 | | | |
| KASUMI-1 | CellTrends, | | RPMI 1640 + 10% FBS |
| | Germany | | |
| KG-1 | CellTrends, | | RPMI 1640 + 10% FBS |
| | Germany | | |
| Mino | ATCC | CRL-3000 | RPMI 1640 + 10% FBS |
| MOLM-13 | CellTrends, | | RPMI 1640 + 10% FBS |
| | Germany | | |
| NU-DHL-1 | DSMZ | ACC 583 | RPMI 1640 + 10% FBS |
| OCI-LY-10 | LM Staudt, | | IMDM + 20% Human |
| | NCI | | plasma + 55 μM BME |
| OCI-LY-19 | DSMZ | ACC 528 | RPMI 1640 + 10% FBS |
| OCI-LY-3 | LM Staudt, | | IMDM + 20% Human |
| | NCI | | plasma + 55 μM BME |
| OCI-LY-7 | LM Staudt, | | IMDM + 20% Human |
| | NCI | | plasma + 55 μM BME |
| Pfeiffer | ATCC | CRL-2632 | RPMI 1640 + 10% FBS |
| RC-K8 | DSMZ | ACC 561 | RPMI 1640 + 10% FBS |
| REC-1 | DSMZ | ACC 584 | RPMI 1640 + 10% FBS |
| RIVA | UM | | RPMI 1640 + 20% |
| | | | Human plasma |
| SC-1 | DSMZ | ACC 558 | RPMI 1640 + 10% FBS |
| SU-DHL-1 | DSMZ | ACC 356 | RPMI 1640 + 10% FBS |
| SU-DHL-10 | DSMZ | ACC 576 | RPMI 1640 + 10% FBS |
| SU-DHL-16 | DSMZ | ACC 577 | RPMI 1640 + 10% FBS |
| SU-DHL-4 | DSMZ | ACC 495 | RPMI 1640 + 10% FBS |
| SU-DHL-5 | DSMZ | ACC 571 | RPMI 1640 + 10% FBS |
| SU-DHL-6 | DSMZ | ACC 572 | RPMI 1640 + 10% FBS |
| SU-DHL-8 | DSMZ | ACC 573 | RPMI 1640 + 10% FBS |
| THP-1 | CellTrends, | | RPMI 1640 + 10% FBS |
| | Germany | | |
| Toledo | ATCC | CRL-2631 | RPMI 1640 + 10% FBS |
| U-2932 | UM | | RPMI 1640 + 20% |
| | | | Human plasma |
| U-2940 | DSMZ | ACC 634 | RPMI 1640 + 10% FBS |
| WSU-DLCL2 | DSMZ | ACC 575 | RPMI 1640 + 10% FBS |
| WSU-FSCCL | DSMZ | ACC 612 | RPMI 1640 + 10% FBS |
| WSU-NHL | DSMZ | ACC 58 | RPMI 1640 + 10% FBS |

ATCC = American Type Culture Collection;
BME = β-mercaptoethanol;
DSMZ = Deutsche Sammlung von Mikroorganismen and Zellkulturen (German resource center for biological material);
FBS = fetal bovine serum;
IMDM = Iscove's Modified Dulbecco's Medium;
NCI = National Cancer Institute;
UM = University of Michigan.

Reverse Phase Protein Array.

Cell pellets were made for 37 cell lines without any compound treatment and a reverse phase protein array (RPPA) was performed as described in Tibes R, et al. Mol Cancer Ther 2006; 5:2512-2521. The RPPA analysis of the 37 hematological cell lines was performed with 262 antibodies. The relative level of each protein in each sample was determined and normalized for protein loading. Protein levels were then transformed to log 2 values and median centered by individual batch.

Gene Expression.

Cells were lysed in RNeasy kit tissue lysis (RLT) buffer (Qiagen; Valencia, Calif.) and total RNA was isolated using RNeasy. Double-stranded cDNA and biotin-labeled cRNA were synthesized using 100 ng of total RNA using Ambion's MessageAmp Premier RNA Amplification Kit. Biotin-labeled complementary RNA (cRNA), at 15 μg, was fragmented and hybridized to each GeneChip Human Genome U133 Plus 2.0 Array. Arrays were then washed by the use of Affymetrix fluidics stations and scanned with the GeneChip Scanner 3000.

Determination of the Growth Inhibitory Effect of Compound 1.

All cell lines were maintained and tested in the culture media indicated in Table 5. The seeding density for each cell line was optimized to ensure assay linearity in 384-well plates. Increasing concentrations of Compound 1 (0.5 nM to 10 μM) were spotted in a 10-point serial dilution fashion (3-fold dilution) in duplicate within the plate via an acoustic dispenser (EDCATS-100) into an empty 384-well plate. The dimethyl sulfoxide (DMSO) concentration was kept constant for a final assay concentration of 0.1% DMSO. Plates were replicated for use against different cell lines and testing periods. After compound plate replication, all plates were sealed (Agilent ThermoLoc) and stored at −20° C. for up to 1 month. Repeat testing of Compound 1 against the control cell line (A549) indicated that there were no significant fluctuations in results, regardless of plate replication sequence or storage time at −20° C., indicating that Compound 1 is stable in pre-spotted plates under the storage conditions indicated for a month. When ready for testing, plates were removed from the freezer, thawed, and unsealed just prior to the addition of the test cells.

Prior to testing, cells were grown and expanded in culture flasks to provide sufficient amounts of starting material. Cells were then diluted to their desired densities and added directly to the compound-spotted 384-well plates. Cells were allowed to grow for 72 hours in 5% CO₂ at 37° C. At the time when exposure of cells to compound began (t₀), initial cell number was assessed via a viability assay (Cell Titer-Glo) by quantifying the level of luminescence generated by adenosine-5′-triphosphate (ATP) present in viable cells. After 72 hours, cell viability of compound-treated cells was assessed via Cell Titer-Glo and read for luminescence.

Cell lines were assayed for growth inhibition by Compound 1 in at least 3 independent tests. A control cell line (A549) was included in each of the assays. The response of the control cell line to the compound was monitored closely to enable comparison of the data generated through the assay period. All data were normalized and presented as a percentage of the growth in DMSO-treated cells. Results were then expressed as a GI₅₀ value, which is the compound concentration required to inhibit cell growth in treated cells to 50% of the growth of the untreated control cells during the 72 hours of treatment. The GI₅₀ value corrects for the cell count at time zero. In addition, the IC₅₀ value of Compound 1 for each cell line was calculated.

Results.

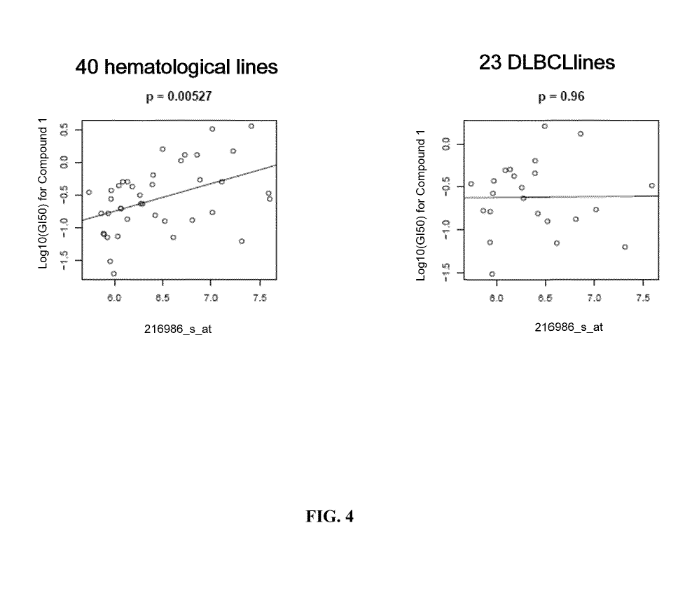

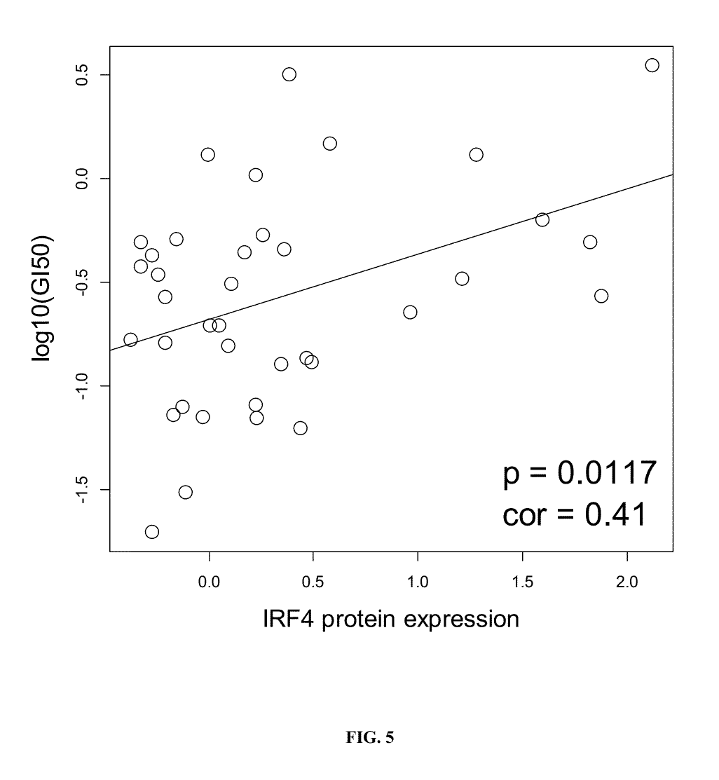

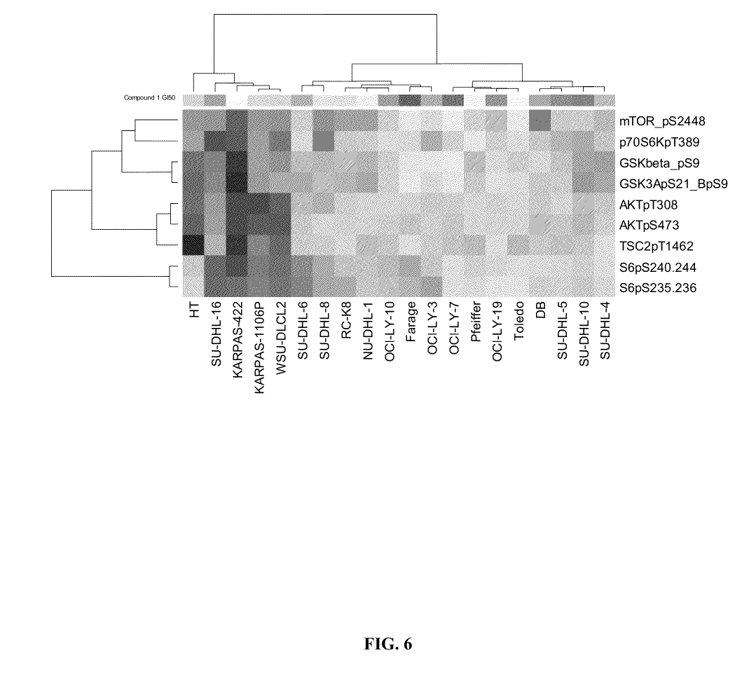

As can be seen in FIG. 4, the IRF4 gene expression level (Probe Set 216986_s_at) negatively correlated with sensitivity to growth inhibition by Compound 1 in 40 hematological cancer cell lines, but not in a subset of the 23 DLBL cell lines included in the hematological cell line panel. Additionally FIG. 5 shows that IRF4 protein levels negatively correlated with sensitivity to growth inhibition by Compound 1 in 37 hematological cancer cell lines. Finally, FIG. 6 shows that the sensitivity to Compound 1 correlated with activation of mTORC1 and mTORC2 in a subgroup of DLBCL lines, as measured via biomarker RPPA (pmTOR S2448, p-p70S6K T389, pGSK3b S9 and S21, pAKT 5473 and T308, pTSC2 T1462, pS6 S240/S244 and S235/S236).

### CONCLUSION

These data indicate that low IRF4 gene and protein levels in hematological cancers correlate with sensitivity to treatment with Compound 1. Additionally, high TORC1 and TORC2 biomarkers correlate with sensitivity to treatment with Compound 1 in DLBCL.

A number of references have been cited, the disclosures of which are incorporated herein by reference in their entirety.

What is claimed is:

1. A method for treating or preventing a breast cancer characterized by a gene mutation, comprising administering an effective amount of a TOR kinase inhibitor to a patient having a breast cancer characterized by a gene mutation, relative to wild type, wherein the gene mutation is a mutation in one or more of RICTOR, TP53 or IGF1R.

2. The method of claim 1, wherein the mutation is a mutation in RICTOR.

3. The method of claim 1, wherein the mutation is a mutation in TP53.

4. The method of claim 1, wherein the mutation is a mutation in IGF1R.

5. The method of any one of claim 1, wherein a further mutation is a mutation in PIK3 CA.

6. The method of any one of claim 1, wherein the breast cancer is ER+.

7. The method of any one of claim 1, wherein the breast cancer is PR+.

8. A method for treating or preventing a breast cancer characterized by a gene mutation, comprising screening a patient's breast cancer for the presence of a gene mutation relative to wild type, and administering an effective amount of a TOR kinase inhibitor to the patient having a cancer characterized by a gene mutation, wherein the gene mutation is a mutation in one or more of RICTOR, TP53 or IGF1R.

9. The method of claim 8, wherein a further gene mutation is a mutation in PIK3CA.

10. A method for predicting response to treatment with a TOR kinase inhibitor in a patient having a breast cancer characterized by a gene mutation, the method comprising: a) obtaining a biological test sample from the patient's cancer; b) obtaining the gene sequence of one or more genes selected from, RICTOR, TP53 or IGF1Rin said biological test sample; c) comparing said gene sequence(s) to the gene sequence(s) of a biological wild-type sample; wherein the presence of a mutation indicates an increased likelihood of response to TOR kinase inhibitor treatment of said patient's cancer.

11. The method of claim 10, wherein a further mutation is a mutation in PIK3CA.

12. A method for predicting therapeutic efficacy of TOR kinase inhibitor treatment of a patient having a breast cancer characterized by a gene mutation, with a TOR kinase inhibitor, the method comprising: a) obtaining a biological test sample from the patient's cancer; b) obtaining the gene sequence(s) of one or more genes selected from RICTOR, TP53 or IGF1R in said biological test sample; c) comparing said gene sequence(s) to the gene sequence(s) of a biological wild-type sample; wherein the presence of a mutation indicates an increased likelihood of therapeutic efficacy of said TOR kinase inhibitor treatment for said patient.

13. The method of claim 12, wherein a further mutation is a mutation in PIK3CA.

14. A method for treating or preventing a breast cancer characterized by a gene mutation, comprising administering an effective amount of a TOR kinase inhibitor to a patient having a breast cancer characterized by a gene mutation, relative to wild type, wherein the gene mutation is a mutation in the gene sequence of AKT1 or a gene amplication mutation in the gene sequence of AKT2.

15. A method for treating or preventing a breast cancer characterized by a gene mutation, comprising screening a patient's breast cancer for the presence of a gene mutation relative to wild type, and administering an effective amount of a TOR kinase inhibitor to the patient having a cancer characterized by a gene mutation, wherein the gene mutation is a mutation in the gene sequence of AKT1 or a gene amplication mutation in the gene sequence of AKT2.

16. A method for predicting response to treatment with a TOR kinase inhibitor in a patient having a breast cancer characterized by a gene mutation, the method comprising: a) obtaining a biological test sample from the patient's cancer; b) obtaining the gene sequence of a gene selected from AKT1 and AKT2 in said biological test sample; c) comparing said gene sequence to the gene sequence of a biological wild-type sample; wherein the presence of a mutation in the gene sequence of AKT1 or the presence of a gene amplification mutation in the gene sequence of AKT2 indicates an increased likelihood of response to TOR kinase inhibitor treatment of said patient's cancer.

17. A method for predicting therapeutic efficacy of TOR kinase inhibitor treatment of a patient having a breast cancer characterized by a gene mutation, with a TOR kinase inhibitor, the method comprising: a) obtaining a biological test sample from the patient's cancer; b) obtaining the gene sequence of a gene selected from AKT1 and AKT2 in said biological test sample; c) comparing said gene sequence to the gene sequence of a biological wild-type sample; wherein the presence of a mutation in the gene sequence of AKT1 or the presence of a gene amplification mutation in the gene sequence of AKT2 indicates indicates an increased likelihood of therapeutic efficacy of said TOR kinase inhibitor treatment for said patient.

18. The method of either of any one of claims 14-17, wherein the mutation is a mutation in the gene sequence of AKT1.

19. The method of either of any one of claims 14-17, wherein the mutation is a gene amplification mutation in the gene sequence of AKT2.

20. A method for treating or preventing a cancer characterized by one or more gene variants, comprising administering an effective amount of a TOR kinase inhibitor to a patient having a cancer characterized by one or more gene variants relative to wild type, wherein the gene variant is a variant in one or more of the genes of FIG. 2, Table 2, or Table 3.

21. The method of claim 20, wherein the cancer is breast cancer, DLBCL, GBM, HCC, MM, NET, or NSCLC.

22. The method of claim 20, wherein the variant is one or more known somatic-variants, likely-somatic variants, rearrangements, variants-of-unknown-significance, or copy-number variants, for example, amplifications or deletions, or a combination thereof.

23. The method of claim 20, wherein the variant is one or more known somatic variants.

24. The method of claim 20, wherein the variant is one or more likely somatic-variants.

25. The method of claim 20, wherein the variant is one or more rearrangements.

26. The method of claim 20, wherein the variant is one or more variants-of-unknown-significance.

27. The method of claim 20, wherein the variant is one or more amplifications.

28. The method of claim 20, wherein the variant is one or more deletions.

29. A method for treating or preventing a cancer characterized by one or more gene variants, comprising screening a patient's cancer for the presence of a gene variant relative to wild type, and administering an effective amount of a TOR kinase inhibitor to the patient having a cancer characterized by one or more gene variants, wherein the gene variant is a variant in one or more genes of Table 2 or Table 3.

30. The method of claim 29, wherein the cancer is breast cancer, DLBCL, GBM, HCC, MM, NET, or NSCLC.

31. The method of claim 29, wherein the variant is one or more known somatic-variants, likely-somatic variants, rearrangements, variants-of-unknown-significance, or copy-number variants, for example, amplifications or deletions, or a combination thereof.

32. A method for predicting response to treatment with a TOR kinase inhibitor in a patient having a cancer characterized by one or more gene variants, the method comprising: a) obtaining a biological test sample from the patient's cancer; b) obtaining the gene sequence of the genes listed in FIG. 2 in said biological test sample; c) comparing said gene sequence(s) to the gene sequence(s) of a biological wild-type sample; wherein the presence of one or more variants in one or more genes from FIG. 2 or Table 2 or Table 3 indicates an increased likelihood of response to TOR kinase inhibitor treatment of said patient's cancer.

33. The method of claim 32, wherein the cancer is breast cancer, DLBCL, GBM, HCC, MM, NET, or NSCLC.

34. A method for predicting therapeutic efficacy of TOR kinase inhibitor treatment of a patient having a cancer characterized by one or more gene variants, with a TOR kinase inhibitor, the method comprising: a) obtaining a biological test sample from the patient's cancer; b) obtaining the gene sequence(s) of the genes listed in FIG. 2 in said biological test sample; c) comparing said gene sequence(s) to the gene sequence(s) of a biological wild-type sample; wherein the presence of one or more variants in one or more genes from FIG. 2, Table 2, or Table 3 indicates an increased likelihood of therapeutic efficacy of said TOR kinase inhibitor treatment for said patient.

35. The method of claim 34, wherein the cancer is breast cancer, DLBCL, GBM, HCC, MM, NET, or NSCLC.
